(19) United States
(12) Patent Application Publication
(10) Pub. No.: US 2021/0171599 A1
(43) Pub. Date: Jun. 10, 2021

(54) COMPOUNDS USEFUL TO TREAT METABOLIC DISORDERS

(71) Applicants: President and Fellows of Harvard College, Cambridge, MA (US); The Brigham and Women's Hospital, Inc., Boston, MA (US)

(72) Inventors: Gokhan S. Hotamisligil, Wellesley, MA (US); Ediz Calay, Boston, MA (US); Amir Tirosh, Ramat Gan (IL); Gurol Tuncman, Chestnut Hill, MA (US); Motohiro Sekiya, Ibaraki (JP)

(73) Assignees: President and Fellows of Harvard College, Cambridge, MA (US); The Brigham and Women's Hospital, Inc., Boston, MA (US)

(21) Appl. No.: 16/937,316

(22) Filed: Jul. 23, 2020

Related U.S. Application Data
(63) Continuation of application No. 16/228,297, filed on Dec. 20, 2018, now abandoned.
Application No. 16/228,297 is a continuation of application No. PCT/US2017/039585, filed on Jun. 27, 2017.
(60) Provisional application No. 62/355,175, filed on Jun. 27, 2016.

(51) Int. Cl. C07K 14/605; C07K 16/26; A61P 3/10; G01N 33/74; G01N 33/92; C07K 16/18

(52) U.S. Cl. CPC C07K 14/605; C07K 16/26; A61P 3/10; G01N 33/74; G01N 33/92; A61K 2039/505; C07K 2317/24; C07K 2317/32; G01N 2333/605; G01N 2500/00; C07K 2317/34; C07K 16/18

(57) ABSTRACT

The present invention provides a method to identify and use compounds for the inhibition of abnormal or dysregulated hepatic glucose production that results in elevated blood glucose levels and associated metabolic disorders. The invention is based on the surprising discovery that the glucagon forms an obligate binding complex with aP2, which is necessary for activation of the glucagon G-coupled protein receptor.

### CROSS-REFERENCE TO RELATED APPLICATIONS

This application is a continuation of U.S. application Ser. No. 16/228,297, filed Dec. 20, 2018, which is a continuation of International Application No. PCT/US2017/039585, filed with the U.S. Receiving Office on Jun. 27, 2017, which claims the benefit of provisional U.S. Application No. 62/355,175, filed Jun. 27, 2016. Each of these applications is hereby incorporated by reference for all purposes.

### STATEMENT OF GOVERNMENTAL INTEREST

This invention was made with United States Government support under contract nos. DK064360 and DK097145 awarded by the National Institutes of Health. The United States Government has certain rights in this invention.

### INCORPORATION BY REFERENCE

The contents of the text file named “15020-017WO1US2_2020-07-23_SEQID_TXT_ST25” which was created on Jul. 23, 2020 and is 62,483 KB in size, are hereby incorporated by reference in their entirety.

### FIELD OF THE INVENTION

The present invention provides compounds and methods of identifying compounds useful in the inhibition of abnormal or dysregulated hepatic glucose production that results in elevated blood glucose levels and associated metabolic disorders.

### BACKGROUND OF THE INVENTION

Obesity, which is characterized by adipose tissue expansion, increases the risk of a cluster of diseases including type 2 diabetes (T2D), non-alcoholic fatty liver disease (NAFLD), and dyslipidemia, which in turn increase the mortality rate from cardiovascular diseases (CVD) (Prospective Studies Collaboration, (2009) The Lancet 373, 1083-1096; Shimomura et al., (2000) Molecular cell 6, 77-86). Obesity is a complex medical disorder of appetite regulation and/or metabolism resulting in excessive accumulation of adipose tissue mass. Obesity is an important clinical problem and is becoming an epidemic disease in western cultures, affecting more than one-third of the US adult population. It is estimated that 97 million adults in the United States are overweight or obese. Obesity is further associated with premature death and with a significant increase in morbidity and mortality from stroke, myocardial infarction, congestive heart failure, coronary heart disease, and sudden death. The primary goals of obesity therapy are to reduce excess body weight, improve or prevent obesity-related morbidity and mortality, and maintain long-term weight loss.

Diabetes is a disease in which the body's ability to produce or respond to the hormone insulin is impaired, resulting in abnormal metabolism of carbohydrates and elevated levels of glucose in the blood and urine. Insulin is a hormone that regulates the movement of glucose into cells. There are two different types of diabetes. With type 1 diabetes (T1D), the pancreas makes no or little insulin. About 1.25 million Americans have T1D and an estimated 40,000 people will be newly diagnosed each year. Type 2 diabetes (T2D), also known as noninsulin-dependent diabetes, is a chronic condition that affects the way the body metabolizes glucose. With type 2 diabetes, the body either resists the effects of insulin or doesn't produce enough insulin to maintain a normal glucose level. Without enough insulin, glucose levels in the blood remain high. About 27.9 million Americans, or 9.3% of the population, have T2D. Diabetes remains the 7th leading annual cause of death in the United States in 2010, with 69,071 death certificates listing it as the underlying cause of death, and a total of 234,051 death certificates listing diabetes as an underlying or contributing cause of death. Complications and co-morbidities of diabetes include hypoglycemia, hyperglycemia, hypertension, dyslipidemia, cardiovascular disease (CVD) myocaridal infarction, stroke, blindness and retinopathies, kidney disease, and amputations.

Both hypoglycemia and hyperglycemia can be damaging to humans and other mammals. The human body has developed a multitude of hormonal responses to fight against hypoglycemia in a manner that sustains the critical functions of the body, such as the brain which exclusively utilizes glucose (Tesfaye N, Seaquist E R. Neuroendocrine responses to hypoglycemia. Ann N Y Acad Sci. 2010 November; 1212:12-28; Marty N, Dallaporta M, Thorens B. Brain Glucose Sensing, Counterregulation, and Energy Homeostasis. Physiology. 2007 Aug. 1; 22(4):241-251; Eigler N, Saccà L, Sherwin R S. Synergistic Interactions of Physiologic Increments of Glucagon, Epinephrine, and Cortisol in the Dog. Journal of Clinical Investigation. 1979 Jan. 1; 63(1):114-123). Dysregulated secretion of these hormones, for example glucagon, contributes significantly to the metabolic abnormalities associated with excessive blood glucose levels (Unger R H, Cherrington A D. Glucagonocentric restructuring of diabetes: a pathophysiologic and therapeutic makeover. J Clin Invest. 2012 Jan. 3; 122(1):4-12). Hyperglycemia, as seen with the development of diabetes, can lead to severe complications, including kidney damage, neurological damage, cardiovascular damage, and damage to the retina or damage to feet and legs. Diabetic neuropathy may be a result of long-term hyperglycemia.

Other complications associated with excess blood glucose levels include polyphagia (frequent hunger, especially pronounced hunger), polydipsia (frequent thirst, especially excessive thirst), polyuria (increased volume of urination (not an increased frequency for urination)), blurred vision, fatigue, poor or impaired wound healing (cuts, scrapes, etc.), tingling in feet or heels, erectile dysfunction, recurrent infections, cardiac arrhythmia, impaired fasting glucose, impaired glucose tolerance, dyslipidemia, obesity, nephropathy, retinopathy, cataracts, stroke, atherosclerosis, diabetic ketoacidosis, hyperglycemic hyperosmolar syndrome, perioperative hyperglycemia, hyperglycemia in the intensive care unit patient, insulin resistance syndrome, and metabolic syndrome.

Current treatment modalities for excessive blood glucose levels, including chronic hyperglycemia, aim at maintaining blood glucose at a level as close to normal as possible through a combination of proper diet, regular exercise, and insulin or other medication such as metformin. Despite these modalities, however, disorders associated with excessive blood glucose levels remain a major global health issue.

Nonalcoholic fatty liver disease (NAFLD), including its more aggressive form nonalcoholic steatohepatitis (NASH), is also increasing in epidemic proportions concurrent with the obesity epidemic (Sowers et al., (2011) Cardiorenal Med. 1:5-12). The dramatic rise in obesity and NAFLD appears to be due, in part, to consumption of a western diet (WD) containing high amounts of fat and sugar (e.g., sucrose or fructose), as fructose consumption in the US has more than doubled in the last three decades (Barrera et al., (2014) Clin. Liver Dis. 18:91-112). NAFLD is characterized by macrovesicular steatosis of the liver occurring in individuals who consume little to no alcohol. The histological spectrum of NAFLD includes the presence of steatosis alone, fatty liver, and inflammation. NASH is a more serious chronic liver disease characterized by excessive fat accumulation in the liver that, for reasons that are still incompletely understood, induces chronic inflammation which leads to progressive fibrosis that can lead to cirrhosis, hepatocellular carcinoma, eventual liver failure and death (Brunt et al., (1999) Am. J. Gastroenterol., 94:2467-2474; Brunt et al., (2001) Semin. Kiver Dis., 21:3-16; Takahashi et al., (2012) World J. Gastroenterol., 18:2300-2308).

Although NASH has become more and more prevalent, now affecting 2-5% of Americans and 2-3% of people in the world (Neuschwander-Tetri et al., (2005) Am. J. Med. Sci., 330:326-3350), its underlying cause is still not clear. It most often occurs in persons who are middle-aged and overweight or obese. Many subjects with NASH have elevated blood lipids (e.g., cholesterol and triglycerides), hyperinsulinemia, insulin resistance, and many have diabetes or prediabetes. Not every obese person or every subject with diabetes has NASH. Furthermore, some subjects with NASH are not obese, do not have diabetes, and have normal blood cholesterol and lipids. NASH can occur without any apparent risk factor and can even occur in children. Thus, NASH is not only caused by obesity. Currently, no specific therapies for NASH exist. The most important recommendations given to persons with this disease are aerobic exercise, manipulations of diet and eating behavior, and reducing their weight.

While there have been continued advancements, there remains an unmet need for more research on the molecular mechanisms that underlie obesity and its medical consequences, as well as new approaches for its treatment. Similarly, there remains a pressing need to identify new compounds and methods of treating and preventing NAFLDs in diabetic and non-diabetic subjects.

It is an object of the invention to identify new compounds and their uses and compositions to treat elevated glucose levels in the blood that contribute to obesity, non-alcoholic fatty liver disease (NAFLD), non-alcoholic steatohepatitis (NASH), and diabetes (Type I and II).

### SUMMARY OF THE INVENTION

The present invention is based on the surprising discovery that glucagon exhibits its activity on the glucagon receptor (GCGR) via a complex in which glucagon is associated with the protein adipocyte fatty acid-binding protein (aP2). As described herein for the first time, it has been discovered that circulating aP2 is an obligatory binding partner of glucagon, supporting glucose metabolism related actions in the liver. The discovery of this protein complex provides a new treatment pathway for modulating glucose metabolism disorders.

As described for the first time herein, circulating aP2 potentiates glucagon's action through the glucagon G-protein coupled receptor, both in cell culture models and in vivo, wherein binding of the glucagon/aP2 complex to the glucagon receptor results in activation of adenylate cyclase, which increases intracellular cAMP, increases glycogenolysis, and increases expression of gluconeogenic enzymes including phosphoenolpyruvate carboxykinase (PEPCK), fructose-1,6-bisphosphate (FBPase-1) and glucose-6-phosphatase (G-6-Pase). In addition, glucagon signaling activates glycogen phosphorylase and inhibits glycogen synthase. This results in hepatic glucose production and elevated blood glucose levels.

Based on this surprising discovery, provided herein are methods of identifying compounds that neutralize the ability of the glucagon receptor agonist glucagon in complex with its obligate binding partner adipocyte lipid binding protein (aP2) from agonizing glucagon receptor signaling. Further provided herein are methods to use the identified compounds to treat a disorder associated with dysregulated or abnormal hepatic glucose production and elevated blood glucose levels by inhibiting the glucagon receptor agonist glucagon in complex with its obligate binding partner adipocyte lipid binding protein (aP2) from binding and agonizing the glucagon receptor.

As a result of this fundamental discovery of the glucagon/aP2 complex, compounds capable of neutralizing the activity of the glucagon/aP2 protein complex, for example an antibody that binds preferentially to the glucagon/aP2 complex, are identified and designed. In one embodiment, the antibody selectively binds to the glucagon/aP2 complex over aP2 or glucagon alone. In one embodiment, the antibody does not bind to GCGR. Such antibodies are useful in the treatment of diseases mediated by glucagon/aP2 agonism of the glucagon receptor.

In a first aspect of the present invention, a method of identifying a compound capable of binding glucagon/adipocyte binding protein complex (glucagon/aP2) is provided comprising:

i. contacting the compound with glucagon in complex with aP2 (glucagon/aP2); and,

ii. determining whether the compound binds to glucagon/aP2.

In one embodiment, the assay is performed in vitro in the absence of cells. The method may further comprise introducing the compound into an assay with aP2 and glucagon, or glucagon/aP2, and GCGR, and, determining whether glucagon/aP2 binds to GCGR, wherein non-binding of glucagon/aP2 to GCGR is indicative of a compound capable of neutralizing glucagon/aP2 agonism of GCGR. In another embodiment, the method comprises introducing the compound into a cellular assay in the presence of aP2 and glucagon, and/or glucagon/aP2, wherein the cellular assay includes a population of cells expressing GCGR, and measuring the biological activity of GCGR. In one embodiment, the cell population expressing GCGR are hepatocytes. In one embodiment, the cell population expressing GCGR are human cells. In one embodiment, the cell population expressing GCGR are human hepatocyte cells. In one embodiment, the compound is further subjected to a competitive binding assay to identify a compound that binds to the glucagon/aP2 complex preferentially over aP2 and/or glucagon.

In a second aspect of the present invention, a method of identifying a compound capable of neutralizing glucagon/aP2 agonism of GCGR is provided comprising:

i. contacting the compound with aP2 and glucagon, and/or glucagon in complex with aP2 (glucagon/aP2);

ii. determining whether the compound binds to aP2, glucagon, or glucagon/aP2;

iii. introducing the compound into an assay with aP2 and glucagon, or glucagon/aP2, and GCGR, and, iv. determining whether glucagon/aP2 binds to GCGR, wherein non-binding of glucagon/aP2 to GCGR is indicative of a compound capable of neutralizing glucagon/aP2 agonism of GCGR. In one embodiment, the assay is performed in vitro in the absence of cells. The method may further comprise introducing the compound into a cellular assay in the presence of aP2 and glucagon, and/or glucagon/aP2, wherein the cellular assay includes a population of cells expressing GCGR, and measuring the biological activity of GCGR. In one embodiment, the cell population expressing GCGR are hepatocytes. In one embodiment, the cell population expressing GCGR are human cells. In one embodiment, the cell population expressing GCGR are human hepatocyte cells. In one embodiment, the compound is further subjected to a competitive binding assay to identify a compound that binds to the glucagon/aP2 complex preferentially over aP2 and/or glucagon.

In a third aspect of the present invention, provided herein is a method of identifying a compound capable of neutralizing glucagon/aP2 agonism of GCGR comprising:

i. contacting aP2 and glucagon, and/or glucagon/aP2 with GCGR in the presence of a compound;

ii. contacting aP2 and glucagon, and/or glucagon/aP2 with GCGR in the absence of a compound; and,

iii. comparing the amount of bound glucagon/aP2 to GCGR in the presence of the compound with the amount of bound glucagon/aP2 to GCGR in the absence of the compound; wherein a reduced amount of glucagon/aP2 binding to GCGR in the presence of the compound is indicative of a compound capable of neutralizing GCGR agonism. In one embodiment, the assay is performed in vitro in the absence of cells. In one embodiment, the compound is further subjected to a competitive binding assay to identify a compound that binds to the glucagon/aP2 complex preferentially over aP2 and/or glucagon.

The method for measuring or identifying binding of the compound to glucagon/aP2 or glucagon/aP2 binding to GCGR is not limited to the described illustrative embodiments. Examples of methods that can be utilized are described further herein and in the Examples provided below, and include biolayer interferometry with direct interaction of aP2 with biotinylated glucagon (See Example 1; FIG. 3A), scintillation proximity assay, in which ¹²⁵I-glucagon interacted with biotinylated aP2 (See Example 1; FIG. 3B), isothermal titration calorimetry, which measures heat liberated from binding events in solution (See Example 1; FIG. 3C) and microscale thermophoresis (See Example 1 and FIGS. 4A-D).

In a fourth aspect of the present invention, provided herein is a method of identifying a compound capable of neutralizing glucagon/aP2 agonism of GCGR comprising:

i. introducing aP2 and glucagon, and/or glucagon/aP2 into a first cellular assay comprising cells expressing GCGR;

ii. determining the biological activity of GCGR in the cells in the first cellular assay;

iii. introducing aP2 and glucagon, and/or glucagon/aP2 into a second cellular assay comprising cells expressing GCGR, wherein the aP2 and glucagon and/or glucagon/aP2 is introduced in the presence of the compound,

iv. determining the biological activity of GCGR in the cells in the second cellular assay; and,

v. comparing the biological activity of GCGR in the first cellular assay with the biological activity of GCGR in the second cellular assay, wherein a reduction in GCGR biological activity in the second cellular assay compared to the GCGR biological activity in the first cellular assay is indicative of a compound that neutralizes glucagon/aP2 agonism of GCGR. In one embodiment, the cell population comprises hepatocytes. In one embodiment, the cell population comprises human cells. In one embodiment, the cell population comprises human hepatocytes. In one embodiment, the compound is further subjected to a competitive binding assay to identify a compound that binds to the glucagon/aP2 complex preferentially over aP2 and/or glucagon.

In a fifth aspect of the present invention, provided herein is a method of identifying a compound capable of neutralizing glucagon/aP2 agonism of GCGR comprising:

i. introducing the compound into a first cellular assay in the presence of aP2 and glucagon, and/or glucagon/aP2 and a cell population comprising cells expressing GCGR, wherein the compound is present at a fixed concentration, and wherein aP2 and glucagon and/or glucagon/aP2 are present at a non-saturated concentration;

ii. determining a biological activity of GCGR in the cell population in the first cellular assay;

iii. introducing the compound into a second cellular assay in the presence of aP2, glucagon, and/or glucagon/aP2 and a cell population comprising cells expressing GCGR, wherein the compound is present at a fixed concentration, and wherein aP2 and glucagon, and/or glucagon/aP2 are present at a saturated concentration;

iv. determining a biological activity of GCGR in the cell population in the second cellular assay; and,

v. comparing the biological activity of GCGR in the first cellular assay with the biological activity of GCGR in the second cellular assay, wherein a reduction in GCGR biological activity in the first cellular assay greater than a reduction in GCGR biological activity in the second cellular assay is indicative of a compound that neutralizes glucagon/aP2 agonism of GCGR. In one embodiment, the cell population comprises hepatocytes. In one embodiment, the cell population comprises human cells. In one embodiment, the cell population comprises human hepatocytes.

In a sixth aspect of the present invention, provided herein is a method of identifying a compound capable of neutralizing glucagon/aP2 agonism of GCGR comprising:

i. introducing the compound into a first cellular assay in the presence of aP2 and glucagon, and/or glucagon in complex with aP2 (glucagon/aP2) and a cell population comprising cells expressing GCGR, wherein the compound is present at a fixed concentration, and wherein aP2 and glucagon and/or glucagon/aP2 are present at a first concentration;

ii. determining a biological activity of GCGR in the cell population in the first cellular assay;

iii. introducing the compound into a series of additional cellular assays in the presence of aP2 and glucagon, and/or glucagon in complex with aP2 (glucagon/aP2) and a cell population comprising cells expressing GCGR, wherein the series of additional cellular assays includes the compound present at a fixed concentration and aP2, glucagon, and/or glucagon/aP2 at serially increasing concentrations compared to the first cellular assay;

iv. determining a biological activity of GCGR in the cell population in the series of additional cellular assays; and,

v. comparing the GCGR biological activity in the first cellular assay with the GCGR biological activity in the series of additional cellular assays, wherein a reduction in GCGR biological activity in the first cellular assay greater than a reduction in GCGR biological activity in the series of additional cellular assays is indicative of a compound that neutralizes glucagon/aP2 agonism of GCGR. In one embodiment, the cell population comprises hepatocytes. In one embodiment, the cell population comprises human cells. In one embodiment, the cell population comprises human hepatocytes.

In a seventh aspect, provided herein is a method of identifying a compound capable of neutralizing glucagon/aP2 agonism of GCGR, comprising:

i. contacting the compound with aP2; and,

ii. determining whether the compound binds to aP2 at amino acid Phe58, Asn60, Glu62 and/or Lys80 of Seq. ID No. 1 or 2;

wherein the binding of the compound to aP2 at amino acid Phe58, Asn60, Glu62 and/or Lys80 of Seq. ID No. 1 or No. 2 is indicative of a compound capable of neutralizing glucagon/aP2 agonism of GCGR. In one embodiment, the assay is performed in vitro in the absence of cells. In one embodiment, the method further comprises introducing the compound into a cellular assay in the presence of aP2 and glucagon, and/or glucagon/aP2, wherein the cellular assay includes a population of cells expressing GCGR, and measuring the biological activity of GCGR. In one embodiment, the cell population expressing GCGR are hepatocytes. In one embodiment, the cell population expressing GCGR are human cells. In one embodiment, the cell population expressing GCGR are human hepatocyte cells.

In an eighth aspect, provided herein is a method of identifying a compound capable of neutralizing glucagon/aP2 agonism of GCGR, comprising:

i. contacting the compound with glucagon; and,

ii. determining whether the compound binds to glucagon at amino acid Phe22, Va123, Gln24, Trp25, Leu26, Met27, Asn28, and/or Thr29 of Seq. ID No. 82;

wherein the binding of the compound to aP2 at amino acid Phe22, Va123, Gln24, Trp25, Leu26, Met27, Asn28, and/or Thr29 of Seq. ID No. 82 is indicative of a compound capable of neutralizing glucagon/aP2 agonism of GCGR. In one embodiment, the assay is performed in vitro in the absence of cells. In one embodiment, the method further comprises introducing the compound into a cellular assay in the presence of aP2 and glucagon, and/or glucagon/aP2, wherein the cellular assay includes a population of cells expressing GCGR, and measuring the biological activity of GCGR. In one embodiment, the cell population expressing GCGR are hepatocytes. In one embodiment, the cell population expressing GCGR are human cells. In one embodiment, the cell population expressing GCGR are human hepatocyte cells.

In a ninth aspect of the present invention, provided herein is a method of neutralizing glucagon/aP2 agonism of GCGR in a subject comprising administering to the subject a compound including but not limited to an antibody that neutralizes the ability of glucagon/aP2 from binding to GCGR. In one embodiment, the compound neutralizes the ability of glucagon to form a complex with aP2 and thus binding to GCGR by binding to aP2 at amino acid Phe58, Asn60, Glu62 and/or Lys80 of Seq. ID No. 1 or No. 2. In one embodiment, the compound neutralizes the ability of glucagon to form a complex with aP2 and thus binding to GCGR by binding to glucagon at amino acid Phe22, Va123, Gln24, Trp25, Leu26, Met27, Asn28, and/or Thr29 of Seq. ID No. 82.

In a tenth aspect of the present invention, provided herein is a method of neutralizing glucagon/aP2 agonism of GCGR in a subject comprising administering to the subject a compound including but not limited to an antibody that inhibits the ability of glucagon/aP2 to form. In one embodiment, the compound neutralizes the ability of glucagon/aP2 from binding to GCGR by binding to the glucagon/aP2 complex preferentially over aP2 and/or glucagon.

In an eleventh aspect of the present invention, provided herein is a method of inhibiting hepatic glucose production in a subject comprising administering to the subject a compound including but not limited to an antibody that neutralizes the ability of a glucagon/aP2 to agonize GCGR, wherein the compound does not directly bind to GCGR. In one embodiment, the compound preferentially binds glucagon/aP2 complex over aP2 and/or glucagon. In one embodiment, the compound does not bind GCGR.

In a twelfth aspect of the present invention, provided herein is a method of inhibiting hepatic selective insulin resistance in a subject comprising administering to the subject a compound including but not limited to an antibody that neutralizes the ability of glucagon/aP2 to agonize GCGR, wherein the compound does not directly bind to GCGR. In one embodiment, the compound preferentially binds glucagon/aP2 complex over aP2 and/or glucagon. In one embodiment, the compound does not bind GCGR.

In a thirteenth aspect of the present invention, provided herein is a method of treating a subject with a disorder mediated by the dysregulation of hepatic glucose production comprising administering to the subject a compound including but not limited to an antibody that neutralizes the ability of a glucagon/aP2 to agonize GCGR, wherein the compound does not directly bind to GCGR. In one embodiment, the compound preferentially binds glucagon/aP2 complex over aP2. In one embodiment, the compound does not directly bind to aP2 and/or glucagon, but preferentially binds to the glucagon/aP2 complex. When administered to a host in need thereof, using a compound that is capable of targeting the interaction of the glucagon/aP2 complex with GCGR provides a decrease in the production of hepatic glucose and decreases blood glucose, resulting in an improved glucose profile. In one embodiment, the disorder mediated by the dysregulation of hepatic glucose production is selected from diet-induced obesity, diabetes (both type 1 and type 2), hyperglycemia, diabetic ketoacidosis, hyperglycemic hyperosmolar syndrome, cardiovascular disease, diabetic nephropathy or kidney failure, diabetic retinopathy, impaired fasting glucose, impaired glucose tolerance, dyslipidemia, obesity, cataracts, stroke, atherosclerosis, impaired wound healing, perioperative hyperglycemia, hyperglycemia in the intensive care unit patient, insulin resistance syndrome, metabolic syndrome, fibrosis, including lung and liver fibrosis, and non-alcoholic fatty liver disease (NAFLD), including nonalcoholic steatohepatitis (NASH). In one embodiment, the disorder is selected from diet-induced obesity, type-II diabetes, and non-alcoholic fatty liver disease (NAFLD). In one embodiment, the disorder is selected from hepatic cellular carcinoma, cirrhosis, glucagonoma, and Necrolytic migratory erythema (NME).

In a fourteenth aspect of the present invention, provided herein is a method of treating a subject with a disorder mediated by the hepatic selective insulin resistance comprising administering to the subject a compound including but not limited to an antibody that neutralizes the ability of glucagon/aP2 to agonize GCGR, wherein the compound does not directly bind to GCGR. In one embodiment, the compound preferentially binds glucagon/aP2 complex over aP2. In one embodiment, the compound does not directly bind to aP2 and/or glucagon, but preferentially binds to the glucagon/aP2 complex. In one embodiment, the disorder is type-II diabetes.

In a fifteenth aspect of the present invention, provided herein is a method of reducing glucose blood levels in a subject comprising administering to the subject a compound including but not limited to an antibody that neutralizes the ability of a glucagon/aP2 to agonize GCGR, wherein the compound does not directly bind to GCGR. In one embodiment, the compound preferentially binds glucagon/aP2 complex over aP2. In one embodiment, the compound does not bind GCGR.

In one embodiment, the antibody, agent or fragment is a loose binder of aP2, for example, with a Kd of greater than 10⁻⁷ M.

In various embodiments, the compound capable of neutralizing glucagon/aP2 agonism of GCGR acts by one or more of (i) preventing or decreasing the binding of glucagon to the glucagon G-protein coupled receptor in a manner that would normally cause intracellular signaling that results in increased intracellular cAMP; (ii) preventing or decreasing the binding of aP2 to the glucagon G-protein coupled receptor in a manner that would normally cause intracellular signaling that results in increased intracellular cAMP; (iii) preventing or decreasing the ability of the glucagon/aP2 protein complex from binding to the receptor and activating downstream signaling; (iv) preventing or decreasing aP2 from allosterically binding to the glucagon G-protein coupled receptor and changing the receptor's three dimensional conformation such that glucagon cannot bind to the receptor, there is reduced glucagon receptor binding, or the binding is altered in a manner that prevents effective intracellular cAMP signaling; (v) preventing or decreasing glucagon from binding to a glucagon/aP2 G-coupled receptor complex in a manner that prevents effective receptor-mediated intracellular cAMP signaling; (vi) preventing or interfering with the glucagon/aP2 complex formation in a manner that prevents effective receptor-mediated intracellular cAMP signaling; and/or (vii) modifying the glucagon/aP2 protein complex by inducing a conformational change that prevents the glucagon/aP2 complex from binding effectively to the glucagon receptor. Any one or a combination of the above are referred to herein as “glucagon/aP2 complex mediated glucagon receptor activity disruption”. In one embodiment, the compound does not bind GCGR.

A compound capable of neutralizing glucagon/aP2 agonism of GCGR can be any compound that prevents glucagon/aP2 from binding to GCGR or disrupts the ability of glucagon/aP2 to agonize GCGR, resulting in a reduction in GCGR biological activity. GCGR biological activity generally refers to any observable effect resulting from the interaction between GCGR and its agonistic binding partner glucagon/aP2. The biological activity may be glucagon/aP2 binding to GCGR, detection of GCGR-mediated intracellular signal transduction; or determination of an end-point physiological effect. Representative, but non-limiting, examples of GCGR biological activity upon agonistic stimulation by glucagon/aP2 include, but are not limited to, signaling and regulation of the processes discussed herein, e.g., inhibition of cyclic AMP formation, reduced hepatic glucose production, decreased glycogenolysis, and reduced expression of gluconeogenic enzymes including phosphoenolpyruvate carboxykinase (PEPCK), fructose-1,6-bisphosphate (FBPase-1), and glucose-6-phosphatase (G-6-Pase). In addition, glucagon signaling activates glycogen phosphorylase and inhibits glycogen synthase. In one embodiment, the compound is a small molecule, a ligand, an antibody, antigen binding agent, or antibody fragment that binds to aP2, glucagon, and or glucagon/aP2 and neutralizes the ability of glucagon/aP2 to agonize GCGR. In one embodiment, the compound does not directly bind to aP2 and/or glucagon, but preferentially binds to the glucagon/aP2 complex. Examples of assays to detect GCGR biological activity are further exemplified in the Example below, and include, assays relating to reduced expression of gluconeogenic enzymes including phosphoenolpyruvate carboxykinase (PEPCK), fructose-1,6-bisphosphate (FBPase-1), and glucose-6-phosphatase (G-6-Pase) (See Example 1; FIGS. 1A and 1B; FIGS. 2A, 2C, and 2D), reduced hepatic glucose production (See Example 1; FIG. 1C), decreased glycogenolysis (See Example 1; FIG. 1D), and inhibition of cyclic AMP formation (See Example 1; FIGS. 1E and 1F).

This adipose tissue-pancreas-liver axis has important implications for the treatment of conditions associated with abnormal glucagon activity or dysregulated glucagon signaling, for example dysregulated hepatic glucose production and elevated blood glucose levels, for example as seen with disorders such as diabetes. By targeting the glucagon/aP2 protein complex, it has been discovered that the activation of the glucagon receptor by glucagon can be modulated, hepatic glucose production can be inhibited, and blood glucose levels normalized in a mouse model of obesity and diabetes. Furthermore, by reducing hepatic glucose production, the counter-regulatory effects of insulin are further heightened. In one embodiment, the glucagon/aP2 protein complex is bound by an antibody or antigen-binding agent such as an antibody fragment to reduce excessive blood glucose levels in a subject, preferably a human, by administering to the subject an antibody, antigen-binding agent or antibody-binding fragment that targets the circulating glucagon/aP2 protein complex. In one embodiment, the formation of the glucagon/aP2 protein complex is disrupted by an aP2 antibody or antigen-binding agent, wherein the antibody interferes with complexion of glucagon and aP2. In one embodiment, the compound preferentially binds to the glucagon/aP2 complex over aP2 and/or glucagon. In one embodiment, the compound does not bind GCGR.

In one embodiment of any of the aspects described above, the antibody selectively binds to the glucagon/aP2 complex over aP2 alone. Methods for identifying preferably binding antibodies are generally known in the art. In one embodiment, provided herein is a method of identifying an antibody that selectively binds glucagon/aP2 over aP2 generally comprising administering to a non-human animal, for example a rabbit, mouse, rat, or goat, a heterologous glucagon/aP2 protein complex, for example human glucagon/aP2, in order to raise antibodies against the heterologous glucagon/aP2 in complex, isolating said antibodies, subjecting said antibodies to one or more binding assays measuring the binding affinity to glucagon/aP2 and aP2 alone, for example a competitive binding assay, wherein antibodies that preferably bind glucagon/aP2 over aP2 are isolated for use to neutralize glucagon/aP2 agonism of GCGR. In one embodiment, the preferably binding glucagon/aP2 antibody comprises CDR regions directed to human glucagon/aP2. In one embodiment, the preferably binding glucagon/aP2 antibody is humanized according to known methods. Methods describing antibody production, including humanizing antibodies, include U.S. Pat. Nos. 7,223,392, 6,090,382, 5,859,205, 6,090,382, 6,054,297, 6,881,557, 6,284,471, and 7,070,775.

A method of preventing or attenuating the severity of a disorder in a host, such as a human, mediated by the glucagon/aP2 protein complex is provided that includes administering an effective amount of an antibody, antigen-binding agent or antibody-binding fragment that targets the circulating glucagon/aP2 protein complex, for example a humanized antibody such as anti-glucagon/aP2 monoclonal antibody or antigen binding agent described herein, resulting in the reduction or attenuation of the biological activity of glucagon. In one embodiment, preferentially binds to the glucagon/aP2 complex over aP2 and/or glucagon alone.

Nonlimiting examples of uses of the described anti-glucagon/aP2 antibodies and antigen binding agents by administering an effective amount to a host in need thereof include one or a combination of:

Reduction of fasting blood glucose levels;

(ii) Reduction of hepatic glucose production;

(iii) Improvement in glucose metabolism;

(iv) Reduction of hyperinsulinemia;

(v) Reduction of liver steatosis; and/or,

(vi) Increase in insulin sensitivity.

In an alternative aspect, also provided herein is a composition comprising glucagon in complex with aP2 bound to an antibody, antigen binding agent, or antibody fragment. In one embodiment, the antibody, antigen binding agent, or antibody fragment is not naturally occurring in humans. In one embodiment, glucagon/aP2 bound to antibody is isolated.

Other features and advantages of the invention will be apparent from the following detailed description and claims.

### BRIEF DESCRIPTION OF THE DRAWINGS

FIGS. 1A-1B are bar graphs illustrating normalized relative gene expression of G6Pc (FIG. 1A) and Pck1 (FIG. 1B) in primary hepatocytes isolated from 12-week old, male C57/BL6ⁱ mice with concurrent or individual stimulations with glucagon (100 nM) and recombinant aP2 (50 μg/mL) as described in Example 1. Experiments were repeated at least two times with similar results. Bar graphs represent mean±standard deviation (s.d.), n=4-5 per group. *P≤0.05, **P≤0.01, ***P≤0.001, ****P≤0.0001, ns P>0.05. Multiple group comparisons were done using one-way ANOVA statistics with Tukey post-test correction.

FIG. 1C is a bar graph illustrating de-novo glucose production which was assayed in serum and glucose free media, with pyruvate (1 μM) and lactate (2 μM) using Amplex red glucose oxidase after 4 hours of stimulation with aP2 and/or glucagon as above (Example 1). Experiments were repeated at least two times with similar results. Bar graphs represent mean±standard deviation (s.d.), n=4-5 per group. *P≤0.05, **P≤0.01, ***P≤0.001, ****P≤0.0001, ns P>0.05. Multiple group comparisons were done using one-way ANOVA statistics with Tukey post-test correction.

FIG. 1D is a line graph illustrating glucose release which was assessed by scintillation counting after 24 hours of stimulation. HepG2-C3A human hepatoma cell line was loaded with glycogen with 5 mM Glucose and 14C—U-glucose (0.5 μCi/well) in the presence of dexamethasone (1 μM) and insulin (10 μM) overnight (Example 1). Experiments were repeated at least two times with similar results. Bar graphs represent mean±standard deviation (s.d.), n=4-5 per group. *P≤0.05, **P≤0.01, ***P≤0.001, ****P≤0.0001, ns P>0.05. Multiple group comparisons were done using one-way ANOVA statistics with Tukey post-test correction.

FIG. 1E is a line graph illustrating luciferase activity that was assayed 4 hours post stimulation in CHO-K1 stably transfected with human GCGR-GFP and 4xcAMP-response element and stimulated in the presence of 10 μg/mL aP2 or glucagon alone in the indicated concentrations (Example 1). Experiments were repeated at least two times with similar results. Bar graphs represent mean±standard deviation (s.d.), n=4-5 per group. *P≤0.05, **P≤0.01, ***P≤0.001, ****P≤0.0001, ns P>0.05. Multiple group comparisons were done using one-way ANOVA statistics with Tukey post-test correction. Graphs show data as mean±standard error of mean (s.e.m.), analyzed using two-way ANOVA.

FIG. 1F is a bar graph illustrating luciferase activity that was assayed 3 hours post stimulation in primary hepatocytes that were infected with cAMP reporter adenovirus (5 M.O.I.) and stimulated as described above. Experiments were repeated at least two times with similar results. Bar graphs represent mean±standard deviation (s.d.), n=4-5 per group. *P≤0.05, **P≤0.01, ***P≤0.001, ****P≤0.0001, ns P>0.05. Multiple group comparisons were done using one-way ANOVA statistics with Tukey post-test correction. Graphs show data as mean±standard error of mean (s.e.m.), analyzed using two-way ANOVA.

FIG. 2A is a bar graph illustrating G6pc promoter activity in HepG2 cells that were transfected with G6Pc promoter driven luciferase in the presence of GCGR or control vectors transiently. Following a 4-hour stimulation, secreted luciferase activity was assayed from media. Graphs show data as mean±s.e.m. All experiments were repeated at least two times with similar results.

FIG. 2B is a bar graph illustrating GCGR binding kinetics, specifically GCGR-ecd binding to biotin-glucagon immobilized to streptavidin sensors in the presence or absence of aP2 that was determined using biolayer interferometry. Graphs show data as mean±s.e.m. All experiments were repeated at least two times with similar results.

FIGS. 2C-2D are bar graphs illustrating normalized relative gene expression of G6Pc (FIG. 2C) and Pck1 (FIG. 2D) in primary hepatocytes that were stimulated with glucagon or glucagon and aP2 in the presence or absence of GCGR allosteric inhibitor L-168,049 (100 nM). Bar graphs represent mean±s.d., n=4-5 per group. *P≤0.05, **P≤0.01, ***P≤0.001, ****P≤0.0001, ns P>0.05. Multiple group comparisons were done using one-way ANOVA statistics with Tukey post-test correction.

FIGS. 2E and 2F are bar graphs illustrating that cells preincubated with an allosteric inhibitor of the glucagon receptor lose the ability to respond to aP2 and glucagon. FIG. 3A illustrates binding affinity (nm) of unlabeled aP2 to biotin glucagon immobilized onto streptavidin probes using biolayer interferometry. Two different concentrations of aP2 were used for binding to a glucagon saturated probe, which was then analyzed using global fitting models. Experiments were repeated at least three times with similar results.

FIG. 3B is a line graph illustrating ¹²⁵I-Glucagon binding to aP2. Biotinylated aP2 was incubated with ¹²⁵I-labeled glucagon in the presence of varying amounts of cold glucagon as competitor. Luminescence emitted from scintillant-coated plates was read and one site competitive inhibitor model was used for curve fittings. Experiments were repeated at least three times with similar results. The graph shows data as mean±s.e.m.

FIG. 3C illustrates results from the binding isotherm of unlabeled aP2 and glucagon in isothermal titration calorimetry experiment. Heat dissipated following each injection of glucagon over a time series is on the left. On the right, the integration of those values to generate the binding curve. Experiments were repeated at least three times with similar results.

FIG. 3D is a bar graph illustrating glucagon immunoreactivity in serum isolated from wild type or aP2 deficient sera that was incubated with monoclonal anti-aP2 coated magnetic beads to pull down aP2 associated complexes, in the presence or absence of excess cold antibody (wild-type sera), or recombinant aP2 reconstitution (200 ng/mL). Following washes, the complex was incubated with HRP conjugated monoclonal-glucagon antibody to detect glucagon signal. Bar graph represents mean±s.d., n=5 per group. *P≤0.05. Paired t-test was used to compare treatments within the groups, One-way ordinary ANOVA was used for multiple group comparisons. Experiments were repeated at least three times with similar results.

FIGS. 3E and 3F are bar graphs that show that glucagon and aP2 can be detected in the serum as a complex using immunoprecipitation methods. Pre-incubation of wild-type serum with CA33 prevents co-immunoprecipitation.

FIG. 3G is a ligand binding curve of aP2 to GCGR-ECD.

FIG. 3H is a ligand binding curve of aP2 to glucagon.

FIG. 3I is a ligand binding curve of glucagon to GCGR-ECD.

FIG. 3J shows the effect of CA33 on the ligand binding curves of aP2 with glucagon.

FIG. 3K is a Western Blot that shows binding of different truncations of glucagon in wild-type and aP2-deficient mice

FIG. 3L is a Western Blot that shows biotinylated glucagon pulls down endogenous aP2 from organ lysates in wild-type and aP2-deficient mice.

FIG. 4A is a binding curve of glucagon to the glucagon receptor from wild-type and GCGR receptor deficient mice in the presence of increasing concentrations of aP2.

FIG. 4B-4D are bar graphs that show that glucagon binding to the GCGR receptor requires aP2 in vivo. ¹²⁵I labeled glucagon was administered to the tail vein of wild-type, aP2 deficient, GCGR deficient and aP2 deficient combined with recombinant aP2. Organs were harvested 5 min. post administration and radiation was counted with liquid scintillation counter.

FIG. 4B is a bar graph that shows ¹²⁵I glucagon incorporation in all of the organs combined.

FIG. 4C is a bar graph that shows ¹²⁵I glucagon incorporation in specific organs harvested.

FIGS. 4D and 4E are bar graphs that show ¹²⁵I glucagon binding of glucagon to isolated membrane-SPA as a function of body weight.

FIG. 4F is a Western Blot that shows that aP2 increases GCGR-ECD (extracellular domain) binding to glucagon.

FIG. 4G is a Western Blot showing aP2 binds to GCGR FIG. 4H is a bar graph that shows aP2 signal in the pellet and supernatant.

FIG. 4I is a Western Blot that shows that aP2 increases GCGR-ECD binding to glucagon.

FIG. 5A is a binding curve determined from the interaction of FABP4 and glucagon obtained from the microscale thermophoresis experiments.

FIG. 5B is a binding curve determined from the interaction of GCGR-ECD and glucagon obtained from the microscale thermophoresis experiments.

FIG. 5C is a binding curve determined from the interaction of GCGR-ECD with tag and hFABP4 obtained from the microscale thermophoresis experiments.

FIG. 5D is a binding curve determined from the interaction of GCGR-ECD without tag and hFABP4 obtained from the microscale thermophoresis experiments.

FIG. 5E is a binding curve determined from the interaction of aP2 and glucagon.

FIG. 6A is a representative model of aP2 and glucagon binding assuming one to one stoichiometric relationship between molecules.

FIG. 6B is a representative model of multiple subunits of aP2 per glucagon molecule.

FIG. 6C illustrates frequency mapping of the models generated using prediction servers to map out highly probable interaction sites between aP2 and glucagon. Darker spots on the map represent higher frequency of appearance of interaction between models submitted. From this analysis, the most probable interaction sites appear to be the C-terminus of glucagon with potential binding sites to aP2 clustering around first alpha helix and around residues 57 and 76 (two beta-barrel loops). Crystal structures used for this analysis was 1GCN for glucagon, and 3P6C and 1LIC for dimeric aP2.

FIG. 7A is a line graph illustrating glycemia (mg/dl) vs. time (minutes) during a glucose tolerance test. The test was performed in 12-week male littermate wild-type or aP2 deficient mice subjected to 4-hour food withdrawal before administering synthetic glucagon (16 μg/kg), aP2 (50 μg) or combination of both. Graphs show data as mean±s.e.m. Experiments were repeated at least three times with similar results.

FIG. 7B is a bar graph illustrating the area under the curve determined from the glucagon tolerance test from FIG. 7A. One-way ordinary ANOVA, with Tukey correction was used for multiple group comparisons. Experiments were repeated at least three times with similar results.

FIG. 7C is a bar graph illustrating glycogen levels that were measured 3 hours after 24-hour fasting and refeeding in the mice from FIG. 7A, to prevent any differences that might be due to postprandial state. Bar graphs represent mean±s.d., n=4-5 per group. *P≤0.05, n.s. not-significant. Unpaired t-test was used to compare treatments between two groups. Experiments were repeated at least three times with similar results.

FIG. 7D is a bar graph illustrating DPP IV activity in the mice from FIG. 7A that was measured using a fluorogenic substrate from blood (Promega). Bar graphs represent mean±s.d., n=4-5 per group. *P≤0.05, n.s. not-significant. Unpaired t-test was used to compare treatments between two groups. Experiments were repeated at least three times with similar results.

FIG. 7E livers were harvested the same way as described in FIG. 7C and were homogenized in RIPA buffer and subjected to western blot following SDS-PAGE. Bar graphs of the normalized signal intensity determined from the western blot are illustrated in the lower panel. Bar graphs represent mean±s.d., n=4-5 per group. *P≤0.05, n.s. not-significant. Unpaired t-test was used to compare treatments between two groups. Experiments were repeated at least three times with similar results.

FIG. 7F is a line graph illustrating blood glucose (mg/dL) vs. time (minutes) in jugular vein catheterized wild-type or aP2 deficient littermate mice that were restrained and infused with somatostatin to rule out any pancreatic effects and inherent differences between genotypes, and basal level of insulin (0.5 mU/kg/min), with pharmacological doses of glucagon (1 mg/kg/min). Wild-type mice responded to glucagon with increase in glycemia, whereas aP2 deficient mice failed to do so. At the end of this 60 min period, aP2 deficient mice required glucose infusion to keep them at euglycaemia whereas wild-type mice had further increase in their glycemia. Graphs show data as mean±s.e.m. All experiments were repeated at least three times with similar results. Experiments were repeated at least three times with similar results.

FIG. 7G is a line graph measuring glucose tolerance of aP2 deficient and wild-type mice treated with either PBS, glucagon, or glucagon and aP2. The x-axis is time and the y-axis is Glucose excursion measured in mg/dL.

FIG. 7H is a bar graph measuring the glucose AUC in aP2 deficient and wild type mice treated with either PBS, glucagon, or glucagon and aP2. The x-axis are the different treatment regimens and the y-axis is AUC.

FIG. 7I is a bar graph that shows the glycogen content at baseline in livers of aP2 deficient mice. The x-axis is wild-type and aP2 deficient mice and the y-axis is glycogen (mg)/dry liver (mg).

FIG. 7J is a bar graph shows the glycogen content at time of euthanasia in livers of aP2 deficient mice. The x-axis is wild-type and aP2 deficient mice and the y-axis is glycogen (mg)/dry liver (mg).

FIG. 7K is a line graph that shows cAMP measurement in wild-type and aP2 deficient mice following glucagon administration. The x-axis is time in minutes following glucagon administration and the y-axis is pmol of cAMP per ug of DNA.

FIG. 8A is a bar graph illustrating HGP (mg/kg/min) levels in conscious, jugular vein catheterized aP2 deficient mice that were restrained and infused with high level of insulin (3 mU/kg/min). To examine the counter-regulatory effects of hormones, the groups were infused with PBS, glucagon (1 mg/kg/min), aP2 and basal glucagon (8 μg/kg/min aP2, 0.1 mg/kg/min) or high levels of glucagon and aP2 together. Only combinatorial administration of high levels of glucagon and aP2 managed to counteract the effects of insulin (last group, as denoted by non-significant suppression of hepatic glucose production). n=6-9 per group. *P≤0.05, **P≤0.01, ***P≤0.001, ****P≤0.0001, ns P>0.05. Multiple group comparisons under clamp conditions were done using one-way ANOVA statistics with Tukey post-test correction. Basal and clamp conditions between groups were compared using repeated measures two-way ANOVA with Sidak correction. Graphs show data as mean±s.e.m.

FIG. 8B is a line graph of a pancreatic clamp experiment in live mice. Under constant infusion of glucagon, there is no glucose production in response to glucagon in aP2 deficient mice. The x-axis is time of glucagon infusion and the y-axis is blood glucose measured in mg/dL.

FIG. 9A is a table listing the binding affinities (Kd(M)) of anti-aP2 monoclonal antibodies (CA33, CA13, CA15, CA23, and H3) to human and mouse aP2 as determined by biomolecular interaction analysis, using a Biacore T200 system.

FIG. 9B is bar graph showing blood glucose levels (mg/dL) at week 0 (open bars) or week 4 (solid bars) in obese mice on a high-fat diet (HFD) treated with vehicle or anti-aP2 monoclonal antibodies CA33, CA13, CA15, CA23, or H3. Blood glucose levels were measured after 6 hours of day-time food withdrawal. * p≤0.05, ** p≤0.01.

FIG. 9C is a line graph showing glucose levels (mg/dL) vs. time (minutes) during a glucose tolerance test (GTT). The test was performed after 2 weeks of treatment in obese mice on HFD with vehicle (diamonds) or anti-aP2 monoclonal antibodies (0.75 g/kg glucose)(CA33; squares)(CA15; triangles). * p<0.05.

FIG. 10A is a bar graph of the signal interaction (nm) as determined by octet analysis for the anti-aP2 antibodies CA33 and H3 against aP2 (black bars) compared to the related proteins FABP3 (gray bars) and FABP5/Mal1 (light gray bars).

FIG. 10B is a table of antibody crossblocking of H3 vs. CA33, CA13, CA15, and CA23 as determined by Biacore analysis. ++=complete blocking; +=partial blocking; −=no crossblocking.

FIG. 10C shows the epitope sequence of aP2 residues involved in the interaction with CA33 and H3, as identified by hydrogen-deuterium exchange mass spectrometry (HDX). Interacting residues are underlined.

FIG. 10D is a superimposed image of the Fab of CA33 co-crystallized with aP2 and the Fab of H3 co-crystallized with aP2.

FIG. 10E is a high-resolution mapping of CA33 epitope on aP2. Interacting residues in both molecules are indicated. Hydrogen bonds are shown as dashed lines. The side chain of K10 in aP2 forms a hydrophobic interaction with the phenyl side chain of Y92.

FIG. 10F is a line graph showing paranaric acid binding to aP2 (relative fluorescence) vs. pH in the presence of IgG control antibody (circles) or CA33 antibody (squares).

FIG. 10G is a graph showing ¹²⁵I glucagon binding as discussed in Example 2. Anti-mouse IgG SPA beads were incubated with serum from wild-type or aP2 knockout mice with ¹²⁵I glucagon. The x-axis shows the glucagon binding of different anti-aP2 antibodies in wild-type and aP2-deficient mice with the background CPM removed.

FIG. 10H is a graph showing ¹²⁵I glucagon binding as discussed in Example 2. Anti-mouse IgG SPA beads were incubated with serum from wild-type or aP2 knockout mice with ¹²⁵I glucagon. The x-axis shows the glucagon binding of different anti-aP2 antibodies in wild-type and aP2-deficient mice as a percentage of input.

FIG. 11A is a bar graph showing fasting blood glucose (mg/dL) in HFD-induced obese aP2−/− mice before (open bars) and after CA33 antibody or vehicle treatment for three weeks (solid bars).

FIG. 11B is a line graph showing glucose levels (mg/dL) in HFD-induced obese aP2−/− mice vs. time (minutes) during a glucose tolerance test (GTT). The test was performed after 2 weeks of vehicle (triangles) or CA33 antibody treatment (squares) in aP2−/− mice.

FIG. 11C is a bar graph showing fasting blood glucose levels (mg/dL) in ob/ob mice before (open bars) and after (solid bars) 3 weeks of CA33 antibody or vehicle treatment (n=10 mice per group). ** p<0.01.

FIG. 11D is a line graph showing glucose levels (mg/dL) in ob/ob mice vs. time (minutes) during a glucose tolerance test (GTT). The test was performed after 2 weeks of vehicle (triangles) or CA33 antibody treatment (squares) in aP2−/− mice. * p<0.05.

FIG. 11E is a line graph showing glucose levels (mg/dL) in ob/ob mice vs. time (minutes) during a glucose tolerance test (GTT). The test was performed after 3 weeks of vehicle (triangles) or CA33 antibody treatment (squares) in aP2−/− mice.

FIG. 11F is a bar graph that shows the glucose AUC levels in ob/ob mice vs. time (minutes) during a glucose tolerance test (GTT). The test was performed after 3 weeks of vehicle (triangles) or CA33 antibody treatment (squares) in aP2−/− mice.

FIG. 12A is a line graph showing glucose levels (mg/dL) vs. time (minutes) in a glucose tolerance test (GTT) following two weeks of selective antibody treatment using high affinity antibodies (CA13, CA15, CA23, and H3) versus vehicle control in high fat diet fed mice.

FIG. 12B is a line graph showing glucose levels (mg/dl) vs. time (minutes) in an insulin tolerance test (ITT) following three weeks of selective antibody treatment using high affinity antibodies (CA13, CA15, CA23, and H3) versus vehicle control in high fat diet fed mice.

FIG. 12C is a line graph showing that aP2 administration to aP2 knockout mice with glucagon rescues glucagon unresponsiveness and preincubation with CA33 and aP2 prevents that.

FIG. 12D is a bar graph showing that aP2 administration to aP2 knockout mice with glucagon rescues glucagon unresponsiveness and preincubation with CA33 and aP2 prevents that.

FIG. 13 provides anti-human glucagon/aP2 complex humanized kappa light chain variable region antibody fragments, wherein the 909 sequence is rabbit variable light chain sequence, and the 909 gL1, gL10, gL13, gL50, gL54, and gL55 sequences are humanized grafts of 909 variable light chain using IGKV1-17 human germline as the acceptor framework. The CDRs are shown in bold/underlined, while the applicable donor residues are shown in bold/italic and are highlighted: 2V, 3V, 63K and 70D. The mutation in CDRL3 to remove a Cysteine residue is shown in bold/underlined and is highlighted: 90A.

FIG. 14A provides anti-human glucagon/aP2 humanized heavy chain variable region antibody fragments, wherein the 909 sequence is rabbit variable heavy chain sequence, and the 909gH1, gH14, gH15, gH61, and gH62 sequences are humanized grafts of 909 variable heavy chain using IGHV4-4 human germline as the acceptor framework. The CDRs are shown in bold/underlined. The two-residue gap in framework 3, in the loop between beta sheet strands D and E, is highlighted in gH1: 75 and 76. Applicable donor residues are shown in bold/italic and are highlighted: 23T, 67F, 71K, 72A, 73S, 74T, 77T, 78V, 79D, 89T, and 91F. The mutation in CDRH2 to remove a Cysteine residue is shown in bold/underlined and is highlighted: 59S. The mutation in CDRH3 to remove a potential Aspartate isomerization site is shown in bold/underlined and is highlighted: 98E. The N-terminal Glutamine residue is replaced with Glutamic acid, and is shown in bold and highlighted: 1E.

FIG. 14B is a bar graph that shows that incubation of aP2 with CA33 blocks the glucagon potentiation effect of aP2 as shown by cAMP response to glucagon here. The x-axis includes different anti-aP2 antibodies and the y-axis is luminescence.

FIG. 14C is a bar graph that shows that a serine mutant (C2S) does not reduce the effect of aP2 as shown by cAMP response to glucagon here. The x-axis is wild-type aP2 and aP2 mutants and the y-axis is luminescence.

FIG. 15 is a line graph illustrating glucose levels (mg/dL) vs. time (minutes) during a glucagon challenge test in mice with diet-induced obesity treated with vehicle or anti-aP2-glucagon monoclonal antibody.

FIG. 16 is a graph illustrating binding affinity (nm) vs. time (seconds) of tethered aP2 to glucagon, monoclonal antibody (mAb), or glucagon plus mAb.

FIGS. 17A-17B are live-cell microscopy image of U2-OS cells that express GCGR-GFP 15 minutes after treatment with aP2 but no glucagon. Without glucagon treatment, minimal internalization of aP2 into cells was observed (Example 6).

FIGS. 17C-17E are live-cell microscopy image of U2-OS cells that express GCGR 30 minutes after treatment with glucagon and aP2. Colocalization of the GCG-GFP signal and the aP2 signal is shown in white. Internalization of aP2 was greatly increased in the presence of glucagon stimulation (Example 6).

FIG. 17F is a graph comparing microscopy images of the islet area of a cell from aP2^{+/+} and aP2^{-/-} cell lines. The difference in pixel count was not significant between the two cells lines. As discussed in Example 7, aP2 deficiency does not cause alpha cell hyperplasia. The two cell lines are shown on the x-axis and pixel count is shown on the y-axis.

FIG. 17G is a graph comparing microscopy images of glucagon-positive staining in a cell from aP2^{+/+} and aP2^{-/-} cell lines (Example 7). The difference in pixel count was not significant between the two cells lines. The two cell lines are shown on the x-axis and pixel count is shown on the y-axis.

FIG. 17H is a graph comparing microscopy images of glucagon-positive staining and islet area in a cell from aP2^{+/+} and aP2^{-/-} cell lines (Example 7). The difference in pixel count was not significant between the two cells lines. The two cell lines are shown on the x-axis and pixel count is shown on the y-axis.

FIG. 17I is a live-cell microscopy image of a cell from the aP2^{+/+} cell line as discussed in Example 7.

FIG. 17J is a live-cell microscopy image of a cell from the aP2^{-/-} cell line. Compared to a cell from the aP2^{+/+} cell line (FIG. 17I), the aP2^{-/-} cell does not exhibit hyperplasia (Example 7). Hyperplasia is a result of glucagon receptor antagonism, a trait distinguishable from aP2 deficiency.

### DETAILED DESCRIPTION OF THE INVENTION

The present invention is based on the discovery that glucagon forms a complex with aP2 as an obligate binding partner which activates the glucagon receptor and, ultimately, promotes hepatic glucose production. In one embodiment, altering the ability of the glucagon-aP2 complex from binding to the glucagon receptor results in disrupting glucagon signaling activity and modulating excess hepatic glucose production, leading to a reduction in blood glucose levels. Such a discovery provides new methods of addressing chronic, elevated blood glucose levels in subjects, for example humans, and new methods for identifying compounds useful in treating disorders associated with chronic, elevated blood glucose levels.

Based on this discovery, methods are provided for identifying compounds capable of interfering with the ability of the glucagon/aP2 complex from agonizing the glucagon receptor (GCGR). Such Compounds are capable of decreasing glucagon signaling activity in a human or other mammal by targeting the glucagon/aP2 protein complex. In one embodiment, the compound is an antibody, antibody-binding agent, or fragment. In one embodiment, the compound preferentially binds glucagon/aP2 complex over aP2 and/or glucagon. In one embodiment, the antibody, agent or fragment is a loose binder of aP2, for example, with a Kd of greater than 10⁻⁷ M.

When administered to a host in need thereof, an antibody, antigen-binding agent or antibody-binding fragment targeting the glucagon/aP2 protein complex neutralizes the activity of glucagon in association with aP2 and provides a decrease in the production of hepatic glucose production, and/or a decrease in blood glucose levels, and/or reduces the occurrence of chronic hyperglycemia. Therefore, by targeting the interaction of aP2 with glucagon, metabolic disorders associated with increased blood glucose levels including, but not limited to, diabetes (both type 1 and type 2), hyperglycemia, diabetic ketoacidosis, hyperglycemic hyperosmolar syndrome, cardiovascular disease, diabetic nephropathy or kidney failure, diabetic retinopathy, impaired fasting glucose, impaired glucose tolerance, dyslipidemia, obesity, cataracts, stroke, impaired wound healing, perioperative hyperglycemia, hyperglycemia in the intensive care unit patient and insulin resistance syndrome can be treated. In certain embodiments, when administered to a subject in need thereof, the antibody or antigen binding agent is useful to reduce fat mass, liver steatosis, improved serum lipid profiles, and/or reduce atherogenic plaque formation or maintenance in a subject. Therefore, the antibodies and antigen binding agents described herein are particularly useful to treat metabolic disorders associated with dysregulated glucagon activity that results in abnormal or excessive blood glucose levels, including, but not limited to, diabetes (both type 1 and type 2), hyperglycemia, obesity, fatty liver disease, or dyslipidemia.

The present invention thus provides at least the following:
(a) A method for identifying compounds which modulate/affect, and preferably neutralize, the agonistic activity of glucagon/aP2 on GCGR for use in a therapy described herein.
(b) A method of modulating glucagon receptor signaling activity by administering an antibody, antigen-binding agent or antibody-binding fragment that targets the glucagon/aP2 protein complex, as described herein, or a described variant or conjugate thereof, that causes glucagon/aP2 complex mediated G-protein coupled receptor activity disruption.
(c) A method of treating a subject, and in particular a human, with an upregulated glucagon-mediated disorder by administering to the subject an antibody, antigen-binding agent or antibody-binding fragment, or a described variant or conjugate thereof, that causes glucagon/aP2 complex mediated G-protein coupled receptor activity disruption.
(d) A method of treating a subject, and in particular a human, with elevated blood glucose levels by administering to the subject an antibody, antigen-binding agent or antibody-binding fragment, or a described variant or conjugate thereof, that causes glucagon/aP2 complex mediated G-protein coupled receptor activity disruption.
(e) A composition comprising glucagon in complex with aP2 bound to an antibody, antigen binding agent, or antibody fragment.

Other features and advantages of the invention will be apparent from the following detailed description and claims.

### General Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques may be performed according to manufacturer's specifications, as commonly accomplished in the art or as described herein. The nomenclatures used in connection with, and the laboratory procedures and techniques of, analytical chemistry, synthetic organic chemistry, and medicinal, and pharmaceutical chemistry described herein are those well-known and commonly used in the art. Standard techniques are used for chemical syntheses, chemical analyses, pharmaceutical preparation, formulation, and delivery, and treatment of patients.

That the present invention may be more readily understood, selected terms are defined below.

The term “host,” “subject,” or “patient” as used herein, typically refers to a human subject, and in particular where a human or humanized framework is used as an acceptor structure. Where another host is treated, it is understood by those of skill in the art that an antibody or antigen binding agent may need to be tailored to that host to avoid rejection or to make more compatible. It is known how to use the CDRs in the present invention and engineer them into the proper framework or peptide sequence for desired delivery and function for a range of hosts. Other hosts may include other mammals or vertebrate species. The term “host,” therefore, can alternatively refer to animals such as mice, monkeys, dogs, pigs, rabbits, domesticated swine (pigs and hogs), ruminants, equine, poultry, felines, murines, bovines, canines, and the like, where the antibody or antigen binding agent, if necessary is suitably designed for compatibility with the host.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments, and polypeptide analogs of a protein sequence. A polypeptide may be monomeric or polymeric.

The term “human aP2 protein” or “human FABP4/aP2 protein”, as used herein refers to the protein encoded by Seq. ID. No. 1, and natural variants thereof, as described by Baxa, C. A., Sha, R. S., Buelt, M. K., Smith, A. J., Matarese, V., Chinander, L. L., Boundy, K. L., Bernlohr, A. Human adipocyte lipid-binding protein: purification of the protein and cloning of its complementary DNA. Biochemistry 28: 8683-8690, 1989.

The term “mouse aP2 protein” or “mouse FAB4P/aP2 protein”, as used herein, refers to the protein encoded by Seq. ID. No. 2, and natural variants thereof. The mouse protein is registered in Swiss-Prot under the number P04117.

“Antigen binding agents” as used herein include single chain antibodies (i.e. a full length heavy chain and light chain); Fab, modified Fab, Fab′, modified Fab′, F(ab′)2, Fv, Fab-Fv, Fab-dsFv, single domain antibodies (e.g. VH or VL or VHH) for example as described in WO 2001090190, scFv, bi, tri or tetra-valent antibodies, Bis-scFv, diabodies, tribodies, triabodies, tetrabodies and epitope-antigen binding agents of any of the above (see for example Holliger and Hudson, 2005, Nature Biotech. 23(9):1126-1136; Adair and Lawson, 2005, Drug Design Reviews-Online 2(3), 209-217). The methods for creating and manufacturing these antibody fragments are well known in the art (see for example Verma et al., 1998, Journal of Immunological Methods, 216, 165-181). The Fab-Fv format was first disclosed in WO2009/040562 and the disulphide stabilised versions thereof, the Fab-dsFv was first disclosed in WO2010/035012. Other antibody fragments for use in the present invention include the Fab and Fab′ fragments described in International patent applications WO2005/003169, WO2005/003170, and WO2005/003171. Multi-valent antibodies may comprise multiple specificities e.g. bispecific or may be monospecific (see for example WO 92/22583 and WO05/113605). One such example of the latter is a Tri-Fab (or TFM) as described in WO92/22583.

A typical Fab′ molecule comprises a heavy and a light chain pair in which the heavy chain comprises a variable region VH, a constant domain CH1 and a natural or modified hinge region and the light chain comprises a variable region VL and a constant domain CL.

A dimer of a Fab′ to create a F(ab′)2 for example dimerization may be through a natural hinge sequence described herein, or derivative thereof, or a synthetic hinge sequence.

The terms “specific binding” or “specifically binding”, as used herein, in reference to the interaction of an antibody, a protein, or a peptide with a second chemical species, mean that the interaction is dependent upon the presence of a particular structure (e.g., an “antigenic determinant” or “epitope” as defined below) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains at least some portion of the epitope binding features of an Ig molecule allowing it to specifically bind to aP2. Such mutant, variant, or derivative antibody formats are known in the art and described below. Nonlimiting embodiments of which are discussed below. An antibody is said to be “capable of binding” a molecule if it is capable of specifically reacting with the molecule to thereby bind the molecule to the antibody.

A “monoclonal antibody” as used herein is intended to refer to a preparation of antibody molecules, which share a common heavy chain and common light chain amino acid sequence, or any functional fragment, mutant, variant, or derivation thereof which retains at least the light chain epitope binding features of an Ig molecule, in contrast with “polyclonal” antibody preparations that contain a mixture of different antibodies. Monoclonal antibodies can be generated by several known technologies like phage, bacteria, yeast or ribosomal display, as well as classical methods exemplified by hybridoma-derived antibodies (e.g., an antibody secreted by a hybridoma prepared by hybridoma technology, such as the standard Kohler and Milstein hybridoma methodology ((1975) Nature 256:495-497).

In a full-length antibody, each heavy chain is comprised of a heavy chain variable region (abbreviated herein as HCVR or VH) and a heavy chain constant region (CH). The heavy chain constant region is comprised of four domains-either CH1, Hinge, CH2, and CH3 (heavy chains γ, a and 6), or CH1, CH2, CH3, and CH4 (heavy chains μ and ε). Each light chain is comprised of a light chain variable region (abbreviated herein as LCVR or VL) and a light chain constant region (CL). The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “antibody construct” as used herein refers to a polypeptide comprising one or more of the antigen binding portions of the invention linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain, for example a human IgA, IgD, IgE, IgG or IgM constant domains. Heavy chain and light chain constant domain amino acid sequences are known in the art.

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having human heavy and light chain variable regions in which one or more of the human CDRs (e.g., CDR3) has been replaced with murine CDR sequences.

The terms “Kabat numbering”, “Kabat definitions” and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31-35 (CDR-H1), residues 50-65 (CDR-H2) and residues 95-102 (CDR-H3) according to the Kabat numbering system. However, according to Chothia (Chothia et al., (1987) J. Mol. Biol., 196, 901-917 (1987)), the loop equivalent to CDR-H1 extends from residue 26 to residue 32. Thus, unless indicated otherwise “CDR-H1” as employed herein is intended to refer to residues 26 to 35, as described by a combination of the Kabat numbering system and Chothia's topological loop definition. For the light chain variable region, the hypervariable region ranges from amino acid positions 24 to 34 for CDRL1, amino acid positions 50 to 56 for CDRL2, and amino acid positions 89 to 97 for CDRL3.

As used herein, the terms “acceptor” and “acceptor antibody” refer to the antibody or nucleic acid sequence providing or encoding at least 80%, at least 85%, at least 90%, at least 95%, at least 98% or 100% of the amino acid sequences of one or more of the framework regions. In some embodiments, the term “acceptor” refers to the antibody amino acid or nucleic acid sequence providing or encoding the constant region(s). In yet another embodiment, the term “acceptor” refers to the antibody amino acid or nucleic acid sequence providing or encoding one or more of the framework regions and the constant region(s). In a specific embodiment, the term “acceptor” refers to a human antibody amino acid or nucleic acid sequence that provides or encodes at least 80%, preferably, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available).

As used herein, the term “CDR” refers to the complementarity determining region within antibody variable sequences. There are three CDRs in each of the variable regions of the heavy chain and the light chain, which are designated CDRH1, CDRH2 and CDRH3 for the heavy chain CDRs, and CDRL1, CDRL2, and CDRL3 for the light chain CDRs. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia, or a mixture thereof, defined CDRs.

As used herein, the term “canonical” residue refers to a residue in a CDR or framework that defines a particular canonical CDR structure as defined by Chothia et al. (J. Mol. Biol. 196:901-907 (1987); Chothia et al., J. Mol. Biol. 227:799 (1992), both are incorporated herein by reference). According to Chothia et al., critical portions of the CDRs of many antibodies have nearly identical peptide backbone conformations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In a preferred embodiment, the donor antibody is an antibody from a species different from the antibody from which the framework regions are obtained or derived. In the context of a humanized antibody, the term “donor antibody” refers to a non-human antibody providing one or more CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, —H2, and —H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

Human heavy chain and light chain acceptor sequences are known in the art.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. See, e.g., Shapiro et al., Crit. Rev. Immunol. 22(3): 183-200 (2002); Marchalonis et al., Adv Exp Med Biol. 484:13-30 (2001). One of the advantages provided by various embodiments of the present invention takes advantage of the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or O -glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

The term “humanized antibody” generally refers to antibodies which comprise heavy and light chain variable region sequences from a non-human species (e.g., a rabbit, mouse, etc.) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. Another type of humanized antibody is a CDR-grafted antibody, in which at least one non-human CDR is inserted into a human framework. The latter is typically the focus of the present invention.

In particular, the term “humanized antibody” as used herein, is an antibody or a variant, derivative, analog or fragment thereof which immuno-specifically binds to an antigen of interest and which comprises a framework (FR) region having substantially the amino acid sequence of a human antibody and a complementarity determining region (CDR) having substantially the amino acid sequence of a non-human antibody. As used herein, the term “substantially” in the context of a CDR refers to a CDR having an amino acid sequence at least 50, 55, 60, 65, 70, 75 or 80%, preferably at least 85%, at least 90%, at least 95%, at least 98% or at least 99% identical to the amino acid sequence of a non-human antibody CDR. In one embodiment, the humanized antibody has a CDR region having one or more (for example 1, 2, 3 or 4) amino acid substitutions, additions and/or deletions in comparison to the non-human antibody CDR. Further, the non-human CDR can be engineered to be more “human-like” or compatible with the human body, using known techniques. A humanized antibody comprises substantially all of at least one, and typically two, variable domains (Fab, Fab′, F(ab′)2, F(ab′)c, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, and CH3, or CH1, CH2, CH3, and CH4 of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgY, IgM, IgG, IgD, IgA and IgE, and any isotype, including without limitation IgA1, IgA2, IgG1, IgG2, IgG3 and IgG4. The humanized antibody may comprise sequences from more than one class or isotype, and particular constant domains may be selected to optimize desired effector functions using techniques well known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond exactly to either the donor antibody or the consensus framework. In a preferred embodiment, such mutations, however, will not be extensive. Usually, at least 50, 55, 60, 65, 70, 75 or 80%, preferably at least 85%, more preferably at least 90%, and most preferably at least 95%, 98% or 99% of the humanized antibody residues will correspond to those of the parental FR and CDR sequences. In one embodiment, one or more (for example 1, 2, 3 or 4) amino acid substitutions, additions and/or deletions may be present in the humanized antibody compared to the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

As used herein, “Vernier” zone refers to a subset of framework residues that may adjust CDR structure and fine-tune the fit to antigen as described by Foote and Winter (1992, J. Mol. Biol. 224:487-499, which is incorporated herein by reference). Vernier zone residues form a layer underlying the CDRs and may impact on the structure of CDRs and the affinity of the antibody.

As used herein, the term “neutralizing” refers to neutralization of biological activity of glucagon/aP2 protein complex activity when a compound specifically interferes with the ability of glucagon/aP2 protein complex to agonize GCGR. Preferably a neutralizing binding protein, for example an antibody, is a binding protein who's binding to aP2, glucagon, and/or glucagon/aP2 protein complex results in neutralization of a biological activity of glucagon/aP2 protein complex. Preferably the neutralizing binding protein decreases a biologically activity of glucagon/aP2 protein complex by at least about 5%, 10%, 15%, 20%, 25%, 30%, 35%, 40%, 50%, 60%, 80%, 85%, or more. Neutralization of a biological activity of glucagon/aP2 protein complex by a neutralizing antibody can be assessed by measuring one or more indicators of glucagon/aP2 protein complex biological activity described herein.

A “neutralizing monoclonal antibody” as used herein is intended to refer to a preparation of antibody molecules, which upon binding to glucagon/aP2 protein complex are able to inhibit or reduce the biological activity of the glucagon/aP2 protein complex activity, that is the ability of the glucagon/aP2 protein complex to activate the glucagon receptor, either partially or fully.

The term “blood glucose level” shall mean blood glucose concentration. In certain embodiments, a blood glucose level is a plasma glucose level. Plasma glucose may be determined in accordance with, e.g., Etgen et al., (2000) Metabolism, 49(5): 684-688 or calculated from a conversion of whole blood glucose concentration in accordance with D'Orazio et al., (2006) Clin. Chem. Lab. Med., 44(12): 1486-1490.

The term “normal glucose levels” refers to mean plasma glucose values in humans of less than about 100 mg/dL for fasting levels, and less than 145 mg/dL for 2-hour postprandial levels or 125 mg/dL for a random glucose. The term “elevated blood glucose level” or “elevated levels of blood glucose” shall mean an elevated blood glucose level such as that found in a subject demonstrating clinically inappropriate basal and postprandial hyperglycemia or such as that found in a subject in oral glucose tolerance test (oGTT), with “elevated levels of blood glucose” being greater than 100 mg/dL when tested under fasting conditions, and greater than about 200 mg/dL when tested at 1 hour.

As used herein, the term “attenuation,” “attenuate,” and the like refers to the lessening or reduction in the severity of a symptom or condition caused by elevated blood glucose levels.

The term “epitope” or “antigenic determinant” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

The term “Kd”, as used herein, is intended to refer to the Affinity (or Affinity constant), which is a measure of the rate of binding (association and dissociation) between the antibody and antigen, determining the intrinsic binding strength of the antibody binding reaction.

As used herein, the term “preferentially binds to glucagon/aP2” means that the compound has a greater affinity for glucagon/aP2 in complex than to aP2, glucagon, and/or GCGR alone. For example, the affinity of the compound for glucagon/aP2 may be on the order of 1-2 greater than its affinity for glucagon, aP2, and/or GCGR alone. Accordingly, in one embodiment, compounds that preferentially bind glucagon/aP2 over aP2, glucagon, and/or GCGR have an affinity that is 1 order of magnitude higher than its binding affinity to aP2, glucagon, and/or GCGR alone. In one embodiment, compounds that preferentially bind glucagon/aP2 over aP2, glucagon, and/or GCGR have an affinity that is 2 orders of magnitude greater than its binding affinity to aP2, glucagon, and/or GCGR alone. In one embodiment, compounds that preferentially bind glucagon/aP2 over aP2, glucagon, and/or GCGR have an affinity that is 3 orders of magnitude greater than its binding affinity to aP2, glucagon, and/or GCGR alone. In the case of an antibody, antigen binding agent, or antibody fragment, affinity can be measured as the equilibrium dissociation constant (K_{D}), a ratio of k_{off}/kₒₙ, between the antibody and its antigen. K_{D} and affinity are inversely related. The K_{D} value relates to the concentration of antibody (the amount of antibody needed for a particular experiment) and so the lower the K_{D} value (lower concentration) the higher the affinity of the antibody. In one embodiment, the compound has a K_{D} value for the glucagon/aP2 complex less than 10⁻⁷, and a K_{D} value for aP2, glucagon, or GCGR greater than 10⁻⁷. In one embodiment, the K_{D} values of the compound bound to glucagon/aP2 is between about 10⁻¹⁰ and 10⁻⁸, while the K_{D} value for the compound bound to glucagon, aP2, and/or GCGR is greater than 10⁻⁸. In one embodiment, the K_{D} value for the compound bound to aP2, glucagon, or GCGR is greater than 10⁻⁷.

The terms “crystal”, and “crystallized” as used herein, refer to an antibody, or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giege, R. and Ducruix, A. Barrett, Crystallization of Nucleic Acids and Proteins, a Practical Approach, 2nd ea., pp. 20 1-16, Oxford University Press, New York, N.Y., (1999).”

As used herein, the term “effective amount” refers to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g. prophylactic or therapeutic agent).

### Glucagon and Glucagon Receptor (GCGR)

Glucagon is a 29-amino acid hormone processed from its pre-pro-form in the pancreatic alpha cells by cell specific expression of prohormone convertase 2 (PC2), a neuroendocrine-specific protease involved in the intracellular maturation of prohormones and proneuropeptides (Furuta et al., (2001) J. Biol. Chem. 276: 27197-27202). In vivo, glucagon is a major counter-regulatory hormone for insulin actions. During fasting, glucagon secretion increases in response to falling glucose levels. Increased glucagon secretion stimulates glucoses production by promoting hepatic glycogenolysis and gluconeogenesis (Dunning and Gerich (2007) Endocrine Reviews 28: 253-283). Thus, glucagon counterbalances the effects of insulin in maintaining normal levels of glucose in animals.

The glucagon amino acid sequence is:

HSQGTFTSDYSKYLDSRRAQDFVQWLMNT (SEQ. ID. No. 82.)

The biological effects of glucagon are mediated through the binding and subsequent activation of a specific cell surface receptor, the glucagon receptor. The glucagon receptor (GCGR) is a member of the secretin subfamily (family B) of G-protein coupled receptors (GPCR). GPCRs are seven-transmembrane receptors located in the cell membrane that bind extracellular substances and transmit signals to an intracellular molecule called a G-protein, which typically either activates the cAMP signal pathway or the phosphatidylinositol signal pathway. The human GCGR is a 477-amino acid sequence GPCR and the amino acid sequence of GCGR is highly conserved across species (Mayo et al., (2003) Pharmacological Rev. 55:167-194). The glucagon receptor is predominately expressed in the liver, where it regulates hepatic glucose output, on the kidney, and on islet β-cells, reflecting its role in gluconeogenesis, intestinal smooth muscle, brain, and adipose tissue. The activation of the glucagon receptors in the liver stimulates the activity of adenylate cyclase and phosphoinositol turnover which subsequently results in increased levels of hepatic glucose production, increased intracellular cAMP, increased glycogenolysis, and increased expression of gluconeogenic enzymes including phosphoenolpyruvate carboxykinase (PEPCK) fructose-1,6-bisphosphate (FBPase-1) and glucose-6-phosphatase (G-6-Pase). In addition, glucagon signaling activates glycogen phophorylase and inhibits glycogen synthase. Studies have shown that higher basal glucagon levels and lack of suppression of postprandial glucagon secretion contribute to diabetic conditions in humans (Muller et al., (1970) NEJM 283: 109-115). Recently, it has been suggested that it is an excess of glucagon activity, rather than insulin deficiency, that is responsible for the diabetic phenotype (Unger R H, Cherrington A D. Glucagonocentric restructuring of diabetes: a pathophysiologic and therapeutic makeover. J Clin. Invest. 2012 Jan. 3; 122(1):4-12).

Adipocyte Protein 2 (aP2)

Human adipocyte lipid binding protein (aP2), also known as fatty-acid binding protein 4 (FABP4), belongs to a family of intra-cellular lipid-binding proteins involved in the transport and storage of lipids (Banzszak et al., (1994) Adv. Protein Chem. 45, 89-151). The aP2 protein is involved in lipolysis and lipogenesis and has been indicated in diseases of lipid and energy metabolism such as diabetes, atherosclerosis, and metabolic syndromes. aP2 has also been indicated in the integration of metabolic and inflammatory response systems. (Ozcan et al., (2006) Science 313(5790):1137-40; Makowski et al., (2005) J Biol Chem. 280 (13):12888-95; and Erbay et al., (2009) Nat Med. 15(12):1383-91). More recently, aP2 has been shown to be differentially expressed in certain soft tissue tumors such as certain liposarcomas (Kashima et al., (2013) Virchows Arch. 462, 465-472).

aP2 is highly expressed in adipocytes and regulated by peroxisome-proliferator-activated receptor-gamma (PPAR-gamma) agonists, insulin, and fatty acids (Hertzel et al., (2000) Trends Endocrinol. Metab. 11, 175-180; Hunt et al., (1986) PNAS USA 83, 3786-3790; Melki et al., (1993) J. Lipid Res. 34, 1527-1534; Distel et al., (1992) J. Biol. Chem. 267, 5937-5941). Studies in aP2 deficient mice (FABP4 indicate protection against the development of insulin resistance associated with genetic or diet-induced obesity and improved lipid profile in adipose tissue with increased levels of C16:1n7-palmitoleate, reduced hepatosteatosis, and improved control of hepatic glucose production and peripheral glucose disposal (Hotamisligil et al., (1996) Science 274, 1377-1379; Uysal et al., (2000) Endocrinol. 141, 3388-3396; Cao et al., (2008) Cell 134, 933-944).

In addition, genetic deficiency or pharmacological blockade of aP2 reduces both early and advanced atherosclerotic lesions in an apolipoprotein E-deficient (ApoE^{-/-}) mouse model (Furuhashi et al., (2007) Nature, June 21; 447 (7147):959-65; Makowski et al., (2001) Nature Med. 7, 699-705; Layne et al., (2001) FASEB 15, 2733-2735; Boord et al., (2002) Arteriosclerosis, Thrombosis, and Vas. Bio. 22, 1686-1691). Furthermore, aP2-deficiency leads to a marked protection against early and advanced atherosclerosis in apolipoprotein E-deficient (ApoE^{-/-}) mice (Makowski et al., (2001) Nature Med. 7, 699-705; Fu et al., (2000) J. Lipid Res. 41, 2017-2023). Hence, aP2 plays a critical role in many aspects of development of metabolic disease in preclinical models.

In the past two decades, the biological functions of FABPs in general and aP2 in particular have primarily been attributed to their action as intracellular proteins. Since the abundance of aP2 protein in the adipocytes is extremely high, accounting for up to a few percent of the total cellular protein (Cao et al., (2013) Cell Metab. 17 (5):768-78), therapeutically targeting aP2 with traditional approaches has been challenging, and the promising success obtained in preclinical models (Furuhashi et al., (2007) Nature 447, 959-965; Won et al., (2014) Nature Mat. 13, 1157-1164; Cai et al., (2013) Acta Pharm. Sinica 34, 1397-1402; Hoo et al., (2013) J. of Hepat. 58, 358-364) has been slow to progress toward clinical translation.

In addition to its presence in the cytoplasm, it has recently been shown that aP2 is actively secreted from adipose tissue through a non-classical regulated pathway (Cao et al., (2013) Cell Metab. 17(5), 768-778; Ertunc et al., (2015) J. Lipid Res. 56, 423-424). The secreted form of aP2 acts as a novel adipokine and regulates hepatic glucose production and systemic glucose homeostasis in mice in response to fasting and fasting-related signals. Serum aP2 levels are significantly elevated in obese mice, and blocking circulating aP2 improves glucose homeostasis in mice with diet-induced obesity (Cao et al., (2013) Cell Metab. 17(5):768-78). Importantly, the same patterns are also observed in human populations where secreted aP2 levels are increased in obesity and strongly correlate with metabolic and cardiovascular diseases in multiple independent human studies (Xu et al., (2006) Clin. Chem. 53, 405-413; Yoo et al., (2011) J. Clin. Endocrin. & Metab. 96, 488-492; von Eynatten et al., (2012) Arteriosclerosis, Thrombosis, and Vas. Bio. 32, 2327-2335; Suh et al., (2014) Scandinavian J. Gastro. 49, 979-985; Furuhashi et al., (2009) Metabolism: Clinical and Experimental 58, 1002-1007; Kaess et al., (2012) J. Endocrin. & Metab. 97, E1943-47; Cabre et al., (2007) Atherosclerosis 195, e150-158). Finally, humans carrying a haploinsufficiency allele which results in reduced aP2 expression are protected against diabetes and cardiovascular disease (Tuncman et al., (2006) PNAS USA 103, 6970-6975; Saksi et al., (2014) Circulation, Cardiovascular Genetics 7, 588-598). WO 2010/102171, titled Secreted aP2 and Methods of Inhibiting Same, to President and Fellows of Harvard University and WO 2016/176656, titled Anti-aP2 Antibodies and Antigen Binding Agents to Treat Metabolic Disorders, to President and Fellows of Harvard University and UCB Biopharma SPRL, describe the use of antibodies targeting circulating aP2 in order to modulate metabolic disorders.

Fatty acid-binding proteins (FABPs) are members of the superfamily of lipid-binding proteins (LBP). Nine different FABPs have to date been identified, each showing relative tissue enrichment: L (liver), I (intestinal), H (muscle and heart), A (adipocyte), E (epidermal), Il (ileal), B (brain), M (myelin) and T (testis). The primary role of all the FABP family members is regulation of fatty acid uptake and intracellular transport. The structures of all FABPs are similar—the basic motif characterizing these proteins is ß-barrel, and a fatty acid ligand or ligands (e.g. a fatty acid, cholesterol, or retinoid) bound in its internal water-filled cavity.

WO2016/176656 to President and Fellows of Harvard College and titled “Anti-aP2 Antibodies and Antigen Binging Agents to Treat Metabolic Disorders” describes monoclonal antibodies directed to aP2 for use in treating disorders such as diabetes, obesity, cardiovascular disease, fatty liver disease, and/or cancer, among others.

The human aP2 protein is a 14.7 kDa intracellular and extracellular (secreted) lipid binding protein that consists of 132 amino acids comprising the amino acid sequence (Seq. ID No. 1) of Table 1. The cDNA sequence of human aP2 was previously described in Baxa, C. A., Sha, R. S., Buelt, M. K., Smith, A. J., Matarese, V., Chinander, L. L., Boundy, K. L., Bernlohr, A. Human adipocyte lipid-binding protein: purification of the protein and cloning of its complementary DNA. Biochemistry 28: 8683-8690, 1989, and is provided in Seq. ID No. 5. The human protein is registered in Swiss-Prot under the number P15090.

TABLE 1
aP2 Protein and cDNA Sequences

| Protein or cDNA | Seq. ID No. | SEQUENCE |
|---|---|---|
| Fatty acid-binding protein, | 1 | MCDAFVGTWKLVSSENFDDYMKEVGVG |
| adipocyte | | FATRKVAGMAKPNMIISVNGDVITIKSEST |
| (FABP4/aP2)[H. sapiens] | | FKNTEISFILGQEFDEVTADDRKVKSTITLD |
| | | GGVLVHVQKWDGKSTTIKRKREDDKLVV |
| | | ECVMKGVTSTRVYERA |
| Fatty acid-binding protein, | 2 | MCDAFVGTWKLVSSENFDDYMKEVGVG |
| adipocyte (FABP4/aP2 | | FATRKVAGMAKPNMIISVNGDLVTIRSEST |
| [M. musculus]) | | FKNTEISFKLGVEFDEITADDRKVKSIITLD |
| | | GGALVQVQKWDGKSTTIKRKRDGDKLVV |
| | | ECVMKGVTSTRVYERA |
| aP2 nuclear localization | 3 | KEVGVGFATRK |
| amino acid sequence | | |
| aP2 fatty acid binding | 4 | RVY |
| domain amino acid | | |
| sequence | | |
| Fatty acid-binding protein, | 5 | ATGTGTGATGCTTTTGTAGGTACCTGGA |
| adipocyte | | AACTTGTCTCCAGTGAAAACTTTGATGA |
| (FABP4/aP2)[H. sapiens] | | TTATATGAAAGAAGTAGGAGTGGGCTTT |
| cDNA | | GCCACCAGGAAAGTGGCTGGCATGGCC |
| | | AAACCTAACATGATCATCAGTGTGAATG |
| | | GGGATGTGATCACCATTAAATCTGAAAG |
| | | TACCTTTAAAAATACTGAGATTTCCTTCA |
| | | TACTGGGCCAGGAATTTGACGAAGTCAC |
| | | TGCAGATGACAGGAAAGTCAAGAGCAC |
| | | CATAACCTTAGATGGGGGTGTCCTGGTA |
| | | CATGTGCAGAAATGGGATGG |
| | | AAAATCAACCACCATAAAGAGAAAACG |
| | | AGAGGATGATAAACTGGTGGTGGAATG |
| | | CGTCATGAAAGGCGTCACTTCCACGAGA |
| | | GTTTATGAGAGAGCATAA |
| Fatty acid-binding protein, | 6 | ATGTGTGATGCCTTTGTGGGAACCTGGA |
| adipocyte (FABP4/aP2 | | AGCTTGTCTCCAGTGAAAACTTCGATGA |
| [M. musculus]) cDNA | | TTACATGAAAGAAGTGGGAGTGGGCTTT |
| | | GCCACAAGGAAAGTGGCAGGCATGGCC |
| | | AAGCCCAACATGATCATCAGCGTAAATG |
| | | GGGATTTGGTCACCATCCGGTCAGAGAG |
| | | TACTTTTAAAAACACCGAGATTTCCTTC |
| | | AAACTGGGCGTGGAATTCGATGAAATCA |
| | | CCGCAGACGACAGGAAGGTGAAGAGCA |
| | | TCATAACCCTAGATGGCGGGGCCCTGGT |
| | | GCAGGTGCAGAAGTGGGATGGAAAGTC |
| | | GACCACAATAAAGAGAAAACGAGATGG |
| | | TGACAAGCTGGTGGTGGAATGTGTTATG |
| | | AAAGGCGTGACTTCCACAAGAGTTTATG |
| | | AAAGGGCATGA |

Methods of Identifying Compounds that Neutralize Glucagon/aP2 Agonism of GCGR

One aspect of the present invention relates to a method for identifying compounds which modulate/affect, and preferably neutralize, the agonistic activity of glucagon/aP2 on GCGR for use in a therapy described herein. In one embodiment, the compounds interact with glucagon, aP2, and/or glucagon/aP2, without directly antagonizing GCGR. Compounds of the present invention may include, by way of non-limiting example, peptides produced by expression of an appropriate nucleic acid sequence in a host cell or using synthetic organic chemistries (e.g., antibodies, antibody fragments, or antigen binding agents), or non-peptide small molecules produced using conventional synthetic organic chemistries well known in the art. Identifying assays may be automated in order to facilitate the identification of a large number of small molecules at the same time.

Methods used for identifying compounds may be cell-based or cell-free. In one embodiment, the screen is cell free, and compounds are screened to determine their ability to interact or bind to aP2, glucagon, and or glucagon/aP2. For example, a compound is contacted with aP2, glucagon, and/or glucagon/aP2 and then an assay is performed to detect binding of the compound to aP2, glucagon, and or glucagon/aP2. In further embodiments, the compound can be contacted with aP2, glucagon, and/or glucagon/aP2 in the presence of GCGR, and the binding of said glucagon/aP2 to GCGR can be measured and compared to the binding of said glucagon/aP2 outside of the presence of the compound.

Assays to detect binding of compounds are well known in the art, for example as described in McFedries, et al, Methods for the Elucidation of Protein-Small Molecule Interactions. Chemistry & Biology (2013); Vol. 20(5):667-673; Pollard, A Guide to Simple and Informative Binding Assays, Mol. Biol. Cell (2010) Vol. 21, 4061-4067, both incorporated herein by reference in their entirety.

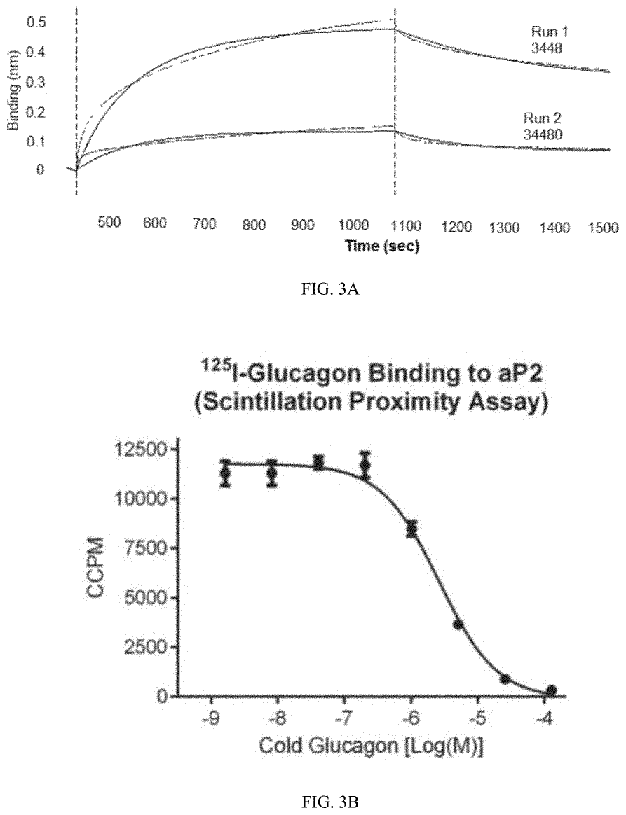

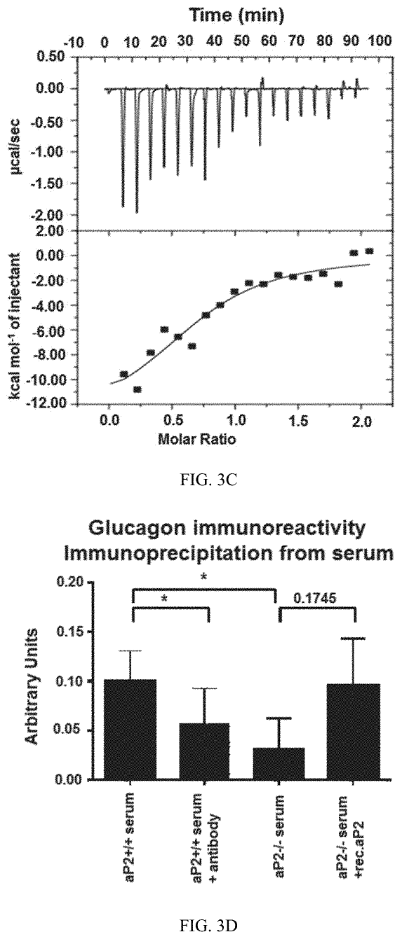

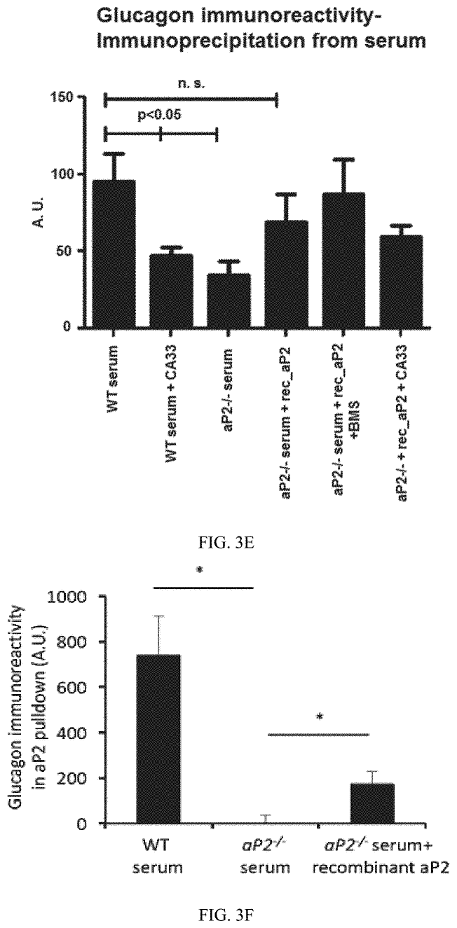

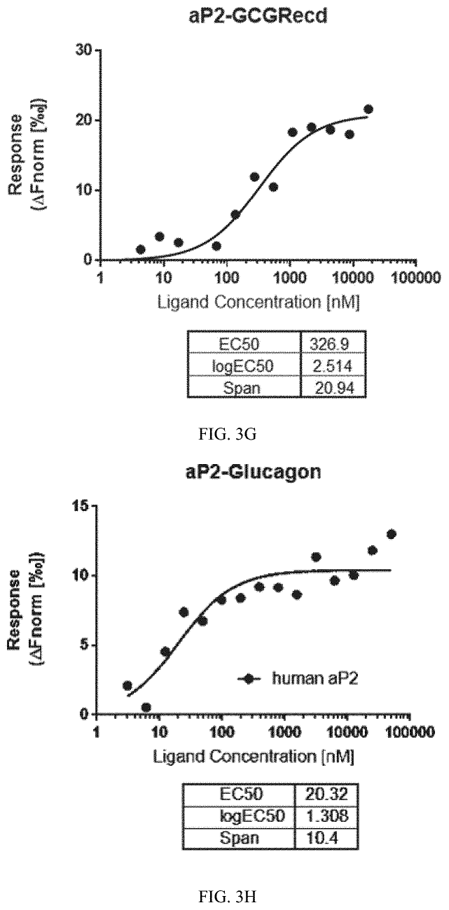

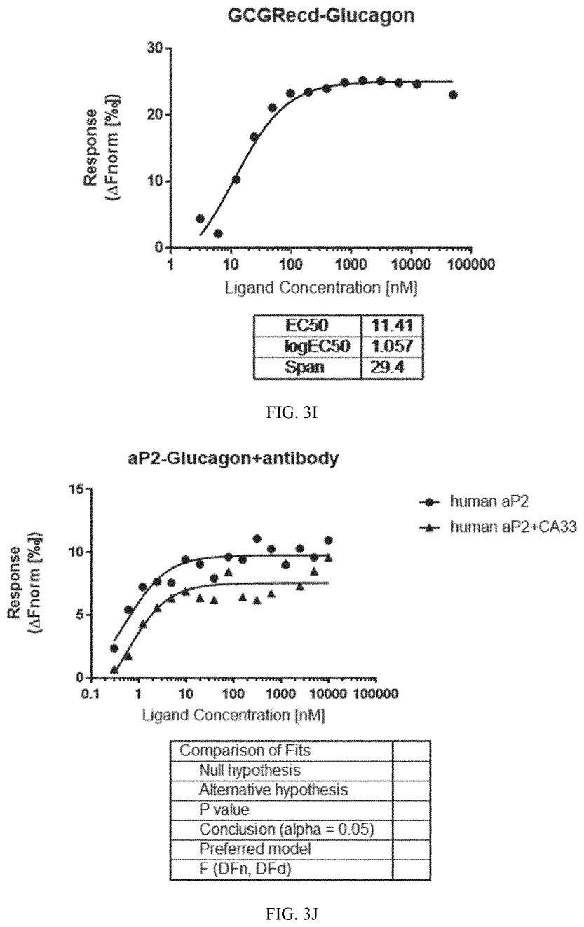

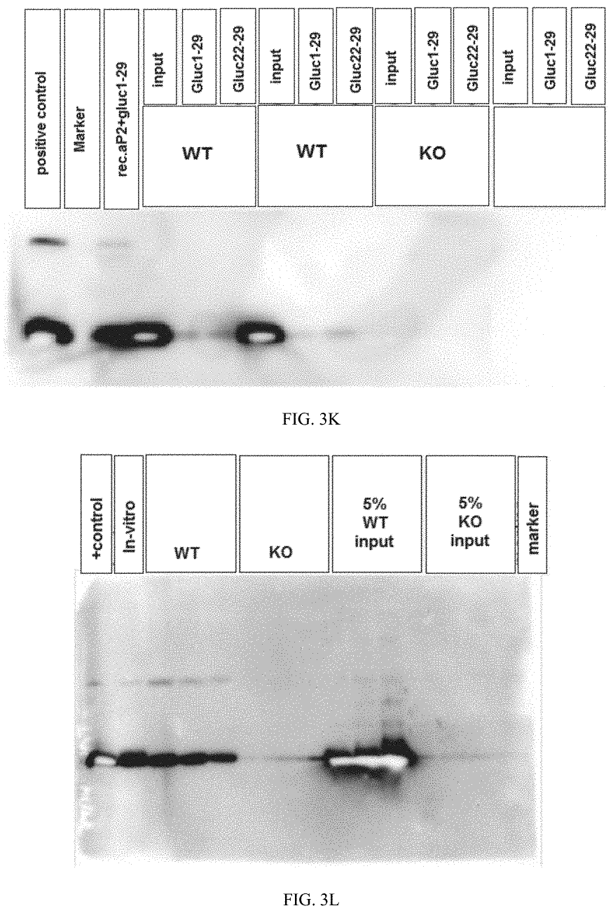

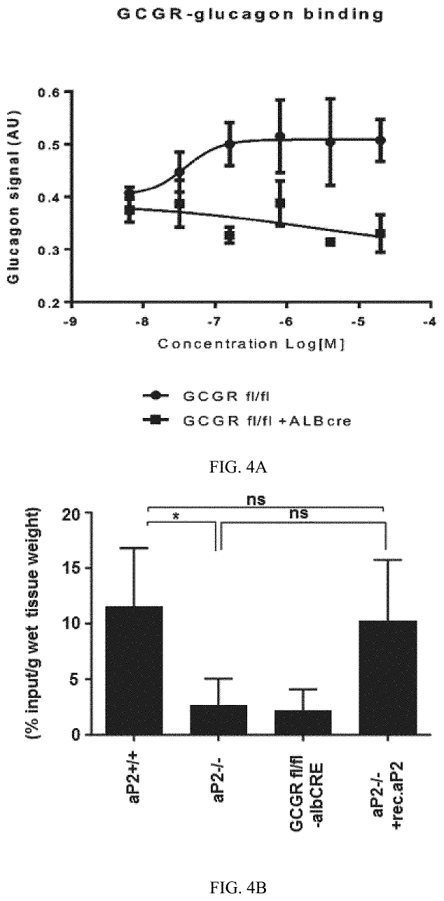

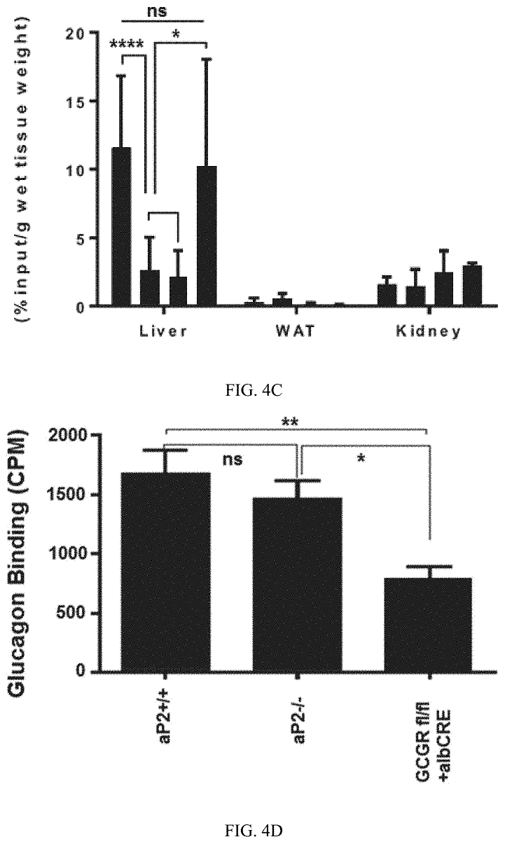

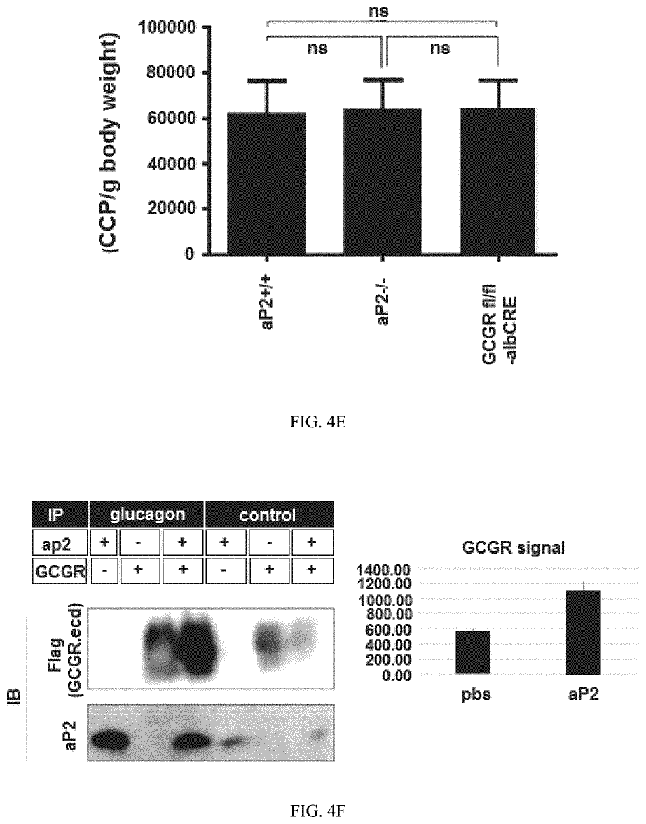

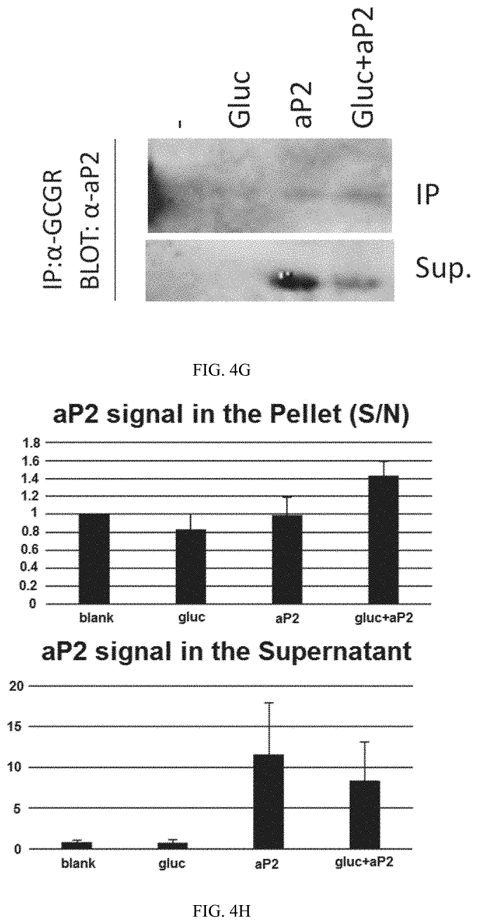

For example, the assay may measure the formation of complexes between aP2, glucagon, and/or glucagon/aP2 and the compound being tested, or examine the degree to which the formation of a complex between glucagon/aP2 and GCGR is interfered with by the compound being tested. Thus, the present invention provides methods of identifying compounds comprising contacting a compound with aP2, glucagon, and/or glucagon/aP2 and assaying (i) for the presence of a complex between aP2, glucagon, and/or glucagon/aP2 and the compound or (ii) for the presence of a complex between glucagon/aP2 and GCGR. In such competitive binding assays, aP2, glucagon, and/or glucagon/aP2 can be labelled. Free glucagon/aP2 is separated from that present in a complex and the amount of free (i.e. uncomplexed) label is a measure of the binding of the compound being tested to aP2, glucagon, and/or glucagon/aP2 or its interference with binding of the glucagon/aP2 to GCGR, respectively. Examples of competitive binding assays that can be utilized include biolayer interferometry with direct interaction of aP2 with biotinylated glucagon (See Example 1; FIG. 3a), scintillation proximity assay, in which ¹²⁵I-glucagon interacted with biotinylated aP2 (See Example 1; FIG. 3b), isothermal titration calorimetry, which measures heat liberated from binding events in solution (See Example 1; FIG. 3c) and microscale thermophoresis (See Example 1; FIGS. 4A-4D).

The identification of a compound capable of neutralizing glucagon/aP2 agonism of GCGR can further be confirmed in additional assays, for example, cell based biological assays or cell-free phosphorylation assays. A sequence for facilitating the detection or purification of bound glucagon/aP2:GCGR complex or glucagon/aP2:compound complex, such as the sequence containing a histidine residue or a continuous sequence thereof (poly-His), a c-Myc partial peptide (Myc-tag), a hemagglutinin partial peptide (HA-tag), a Flag partial peptide (Flag-tag), a glutathione-S-transferase (GST), a maltose-binding protein (MBP), botinylation, labeling with a fluorescent substance (such as a fluorescein), an Eu chelate, a chromophore, a luminophore, an enzyme, or a radioisotope (such as ¹²⁵I or tritium); or binding of a compound having a hydroxysuccinimide residue, a vinyl pyridine residue, etc. for facilitating the binding to a solid phase (such as a container or a carrier), may be introduced into the amino terminal, the carboxy terminal, or an intermediate region of the amino acid sequence of aP2, GCGR, glucagon, or the compound, if the compound is an antibody or fragment thereof, and such proteins can be used during the screen.

In one embodiment, the present invention provides a method of identifying compounds capable of neutralizing glucagon/aP2 agonism of GCGR utilizing eukaryotic cells expressing GCGR and analyzing the biological effects the compound has on glucagon/aP2 agonism of GCGR. Such cells, either in viable or fixed form, can be used for standard binding assays. For example, the assay may measure the formation of complexes between glucagon/aP2 and GCGR in the presence of the compound, or examine the degree to which biological activity of GCGR in the presence of glucagon/aP2 is interfered with by the compound. Thus, the present invention provides methods of identifying compounds comprising contacting a compound and aP2, glucagon, and/or glucagon/aP2 and assaying (i) for the presence of a complex between the glucagon/aP2 and GCGR or (ii) for inhibition of glucagon/aP2 agonism on GCGR by measuring the biological effect of GCGR. The influence of the compound on a biological activity of GCGR can be determined by methods well known in the art. In such activity assays the biological activity of GCGR is typically monitored by provision of a reporter system. For example, this may involve provision of a natural or synthetic substrate that generates a detectable signal in proportion to the degree to which it is acted upon by the biological activity of GCGR stimulation, for example, the measurement of cyclic AMP formation, the expression levels of gluconeogenic enzymes including phosphoenolpyruvate carboxykinase (PEPCK), fructose-1,6-bisphosphate (FBPase-1), and glucose-6-phosphatase (G-6-Pase), the measurement of glycogen phosphorylase and glycogen synthase, hepatic glucose production, and glycogenolysis.

The cell-based assay includes a cell that expresses GCGR, either endogenously or recombinantly. GCGR, as expressed, may be in the state of a monomer, a dimer or a multimer, as long as it is capable of eliciting a measurable biological affect upon stimulation by glucagon/aP2 binding. GCGR may be derived from any organism such as human beings, mice, rat, cattle, pig, or rabbit. In one embodiment, the GCGR expressed is of human nature and derived from the endogenous human GCGR protein (UniProtKB—P47871 (GLR_HUMAN)). GCGR may be extracted from a cell or tissue existing in nature, and may be extracted from a cell or tissue which expresses the subunit by a genetic engineering procedure. GCGR may be purified or unpurified. GCGR produced by a genetic engineering procedure having a reported amino acid sequence or a variant amino acid sequence obtained by genetic mutation can be used as long as it substantially maintains the activity.

In one embodiment, the assay is a cell-free assay and the compound is brought into contact with aP2, glucagon, and/or glucagon/aP2 in a liquid phase, or alternatively aP2, glucagon, and/or glucagon/aP2 is fixed to a solid phase (such as a column) and then contacted with the compound. For example, the glucagon/aP2 may be fixed to the solid phase by biotin/streptavidin, by using a reactable amino group, such as a hydroxysuccinimide group, by using a reactive carboxyl group on a surface, such as a hydrazine group, or by using a group reactable with a thiol group on a surface, such as a vinyl pyridine group. For example, glucagon/aP2 may be fixed to the solid phase (such as a column) by attaching to a solid phase composed of a polystyrene resin or a glass using the electrostatic attractive force or the intermolecular force, by binding glucagon/aP2 to a solid phase obtained by immobilizing an antibody against an amino acid sequence added to aP2 and/or glucagon/aP2 (such as poly-His, Myc-tag, HA-tag, Flag-tag, GST, or MBP), by binding glucagon/aP2 attached with poly-His to a solid phase having on the surface a metal chelate, by binding glucagon/aP2 attached with GST to a solid phase having on the surface a glutathione, or by binding glucagon/aP2 attached with MBP to a solid phase having on the surface a sugar such as maltose. Glucagon/aP2 may also be fixed to the solid phase by another generally known method.

The contacting step of the compound with glucagon/aP2 may be conducted, for example, by mixing a solution containing them. Alternatively, if, for example, glucagon/aP2 or, alternatively the compound, is fixed to a solid phase such as a column, tube, or a multi-well plate, adding a solution containing the non-bound compound.

Compounds that are found to bind to aP2, glucagon, and/or glucagon/aP2 can be further tested in a cell free assay to determine the ability to prevent glucagon/aP2 binding of GCGR. For example, in one embodiment, the binding of glucagon/aP2 contained in a liquid phase or fixed to a solid phase (such as a column, container, or a carrier) with GCGR can be measured in the presence and absence of the compound respectively, and the change of the binding depending on the addition of the compound is observed, to evaluate the inhibitory effect of the compound on the binding of glucagon/aP2 to GCGR. The binding of glucagon/aP2 to GCGR may be measured with or without separating them. For example, glucagon/aP2, GCGR, and glucagon/aP2 bound to GCGR (glucagon/aP2:GCGR) may be separated by a gel filtration method, a column method using an affinity resin, an ion exchange resin, etc., a centrifugation method, or a washing method. For example, the amount of glucagon/aP2 bound to GCGR, or amount of glucagon/aP2 unbound to GCGR, may be measured after separating glucagon/aP2 bound to GCGR, GCGR, and unbound glucagon/aP2 from the liquid phase by the gel filtration method or the column method (an affinity resin, an ion exchange resin, etc.). In the case of fixing glucagon/aP2 to the solid phase (such as a column, container or carrier), the solid phase (such as the column, container, or carrier) may be separated from a liquid phase by centrifugation, washing, distributive segregation, precipitation, etc., both in the presence and absence of the compound. In this case, the binding amount may be obtained directly by measuring the amount of GCGR bound to the separated solid phase (such as the column, container or carrier), or indirectly by measuring the amount of GCGR remaining in the liquid phase, both in the presence and absence of the compound. The GCGR in the liquid phase may be separated by an immunoprecipitation method using a protein or an antibody specifically reactable with GCGR, as well as a gel filtration method, a column method using an affinity resin, an ion exchange resin, etc., a centrifugation method, or a washing method. The binding amount of glucagon/aP2 and GCGR may be obtained directly by measuring the amount of the separated glucagon/aP2 or GCGR, or indirectly by measuring the amount of glucagon/aP2 or GCGR contained in a fraction separated from fractions containing the bound glucagon/aP2 and GCGR.

In the methods above, the amount of glucagon/aP2:GCGR and glucagon/aP2:compound contained in a solution may be measured using, for example, glucagon/aP2 labeled with biotin, a radioisotope, a fluorophore, a chromophore, or a chemiluminescent moiety. For example, the amount of the biotin-labeled glucagon/aP2 may be measured by using a protein capable of binding to the biotin with high affinity such as avidin, streptavidin, or a variant protein thereof (hereinafter referred to as the avidins) such that avidins are labeled with the radioisotope, the fluorophore, the luminophore, or the enzyme, which can be easily detected, and bound to the biotin-labeled compound. The radioactive substance may be measured using a common radiation measuring apparatus such as a scintillation counter, a gamma counter, or a GM meter. The fluorophore, the chromophore, and the luminophore may be measured using a fluorescence measuring apparatus, an absorptiometer, and a luminescence measuring apparatus respectively. The amount of the enzyme-labeled compound can be easily measured using a compound that is converted by the enzyme to a chromogenic, fluorescent, or luminescent compound.

The amount of the glucagon/aP2 bound or unbound contained in a solution may be measured as follows. For example, the glucagon/aP2 labeled with the biotin, the fluorescent substance (such as the fluorescein), the Eu chelate, the chromophore, the luminophore, or the radioisotope (such as ¹²⁵I or tritium) may be measured in the same manner as above. The biotinylated glucagon/aP2 may be measured by an immunoprecipitation method, a Western blot method, a solid-phase enzyme immunoassay (an enzyme-linked immuno-sorbent assay: ELISA), or a sandwich assay such as a radioimmunoassay, by using a protein such as streptavidin; an antibody against glucagon/aP2; an antibody against an amino acid sequence added to aP2 (such as poly-His, Myc-tag, HA-tag, Flag-tag, GST, or MBP); a molecule having a metal chelate against a poly-His-added glucagon/aP2; a molecule having a glutathione against a GST-added glucagon/aP2; a molecule having a sugar such as maltose against an MBP-added glucagon/aP2; etc.

In a more specific example, glucagon/aP2 having a Myc-tag sequence is contacted with a tritium-labeled GCGR using a 96-multi-well plate in the presence/absence of a compound in the presence of an anti-Myc antibody (a mouse-derived monoclonal antibody) and an anti-mouse immunoglobulin antibody-fixed SPA bead, and after a certain period, the binding amount of the glucagon-aP2 and the tritium-labeled GCGR is measured using a scintillation counter, and the counted values obtained in the presence/absence of the compound are compared, whereby the inhibitory effect of the compound against the binding of glucagon/aP2 to GCGR is measured.

The method for measuring the inhibitory activity of the compound against the binding of glucagon/aP2 to GCGR is not particularly limited. For example, the inhibitory activity may be measured by fixing glucagon/aP2 to the solid phase; contacting glucagon/aP2 with GCGR in the presence or absence of a compound; and measuring the amount of the GCGR bonded to glucagon/aP2 on the solid phase to measure the inhibitory activity of the compound against the binding of glucagon/aP2 to GCGR. Alternatively, the method comprises fixing the GCGR to the solid phase; contacting the GCGR with glucagon/aP2 in the presence or absence of a compound; and measuring the amount of glucagon/aP2 bonded to the solid phase to measure the inhibitory activity of the compound against the binding of glucagon/aP2 to GCGR. A further alternative includes contacting the glucagon/aP2 with GCGR in the presence or absence of a compound; and measuring the binding amount of the glucagon/aP2 and GCGR to measure the inhibitory activity of the compound against the binding of glucagon/aP2 to GCGR. In any of the methods above for example, the binding amount obtained by the contact in the presence of the compound may be compared with the binding amount obtained by the contact in the absence of the compound, to measure the inhibitory activity of the compound against the binding of glucagon/aP2 to GCGR.

In one embodiment, the assay is a cell-based assay, wherein the method for identifying the compound by measuring the inhibitory activity of the compound against the binding of glucagon/aP2 to GCGR uses a cell, a tissue, or an extract thereof containing GCGR. The cell or tissue substantially containing GCGR may be derived from any organism and may be any cell or tissue, although preferably a mammal cell or tissue, including a human cell or tissue. The cell or tissue may be one in which GCGR is endogenously expressed or is expressed by a genetic engineering procedure.

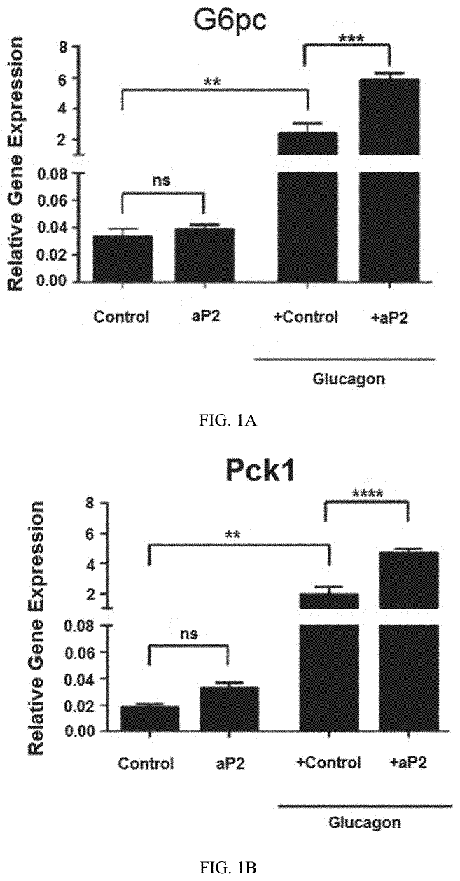

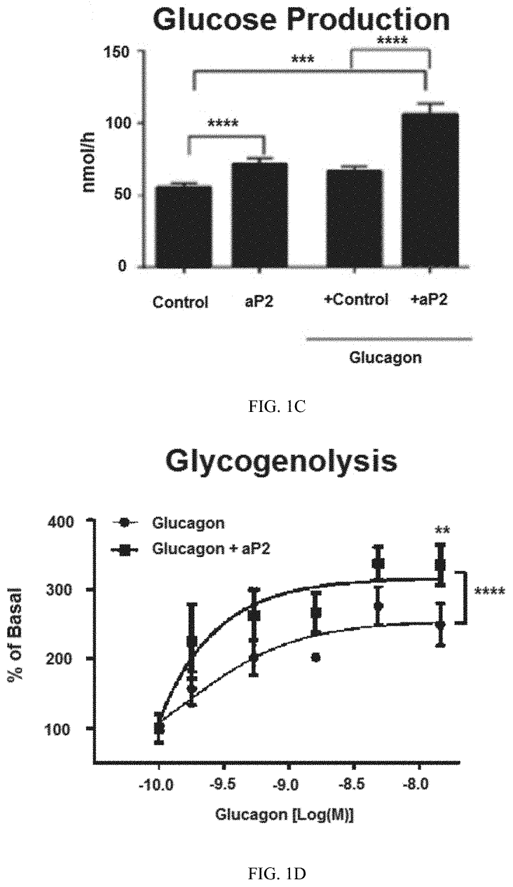

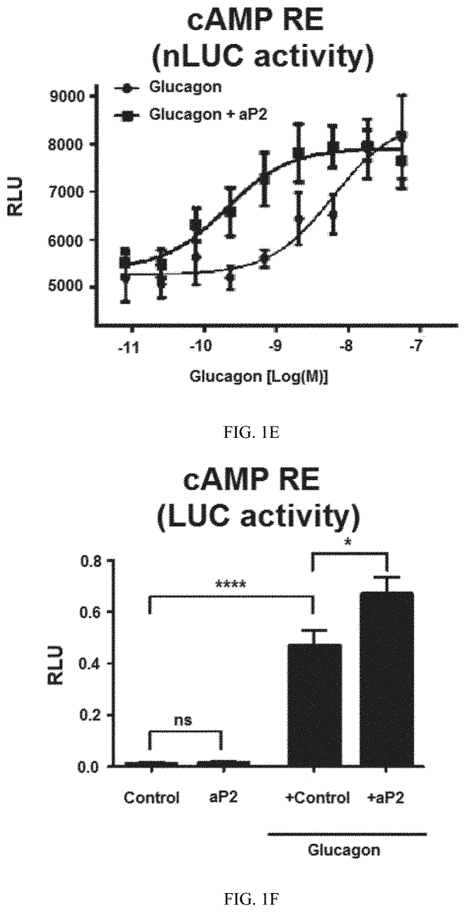

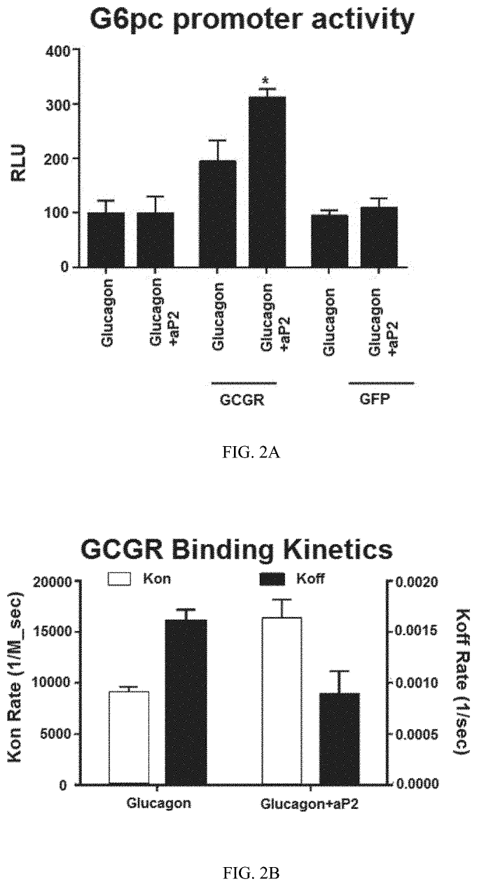

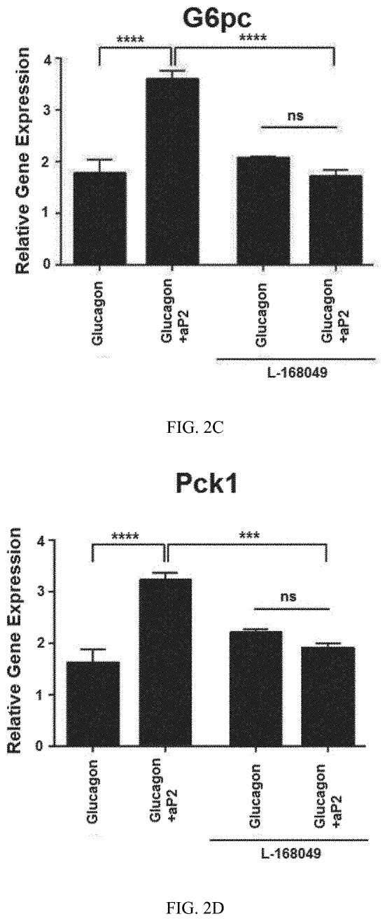

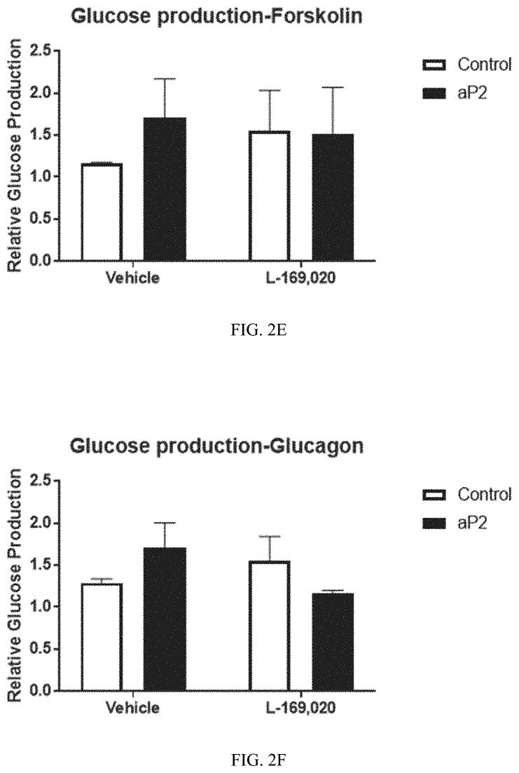

In one embodiment, a cell population expressing GCGR is contacted with a solution comprising glucagon, aP2, and/or glucagon/aP2, and the biological activity of GCGR is measured in the presence and absence of a compound. GCGR biological activity generally refers to any observable effect resulting from the interaction between the GCGR and its agonistic binding partner glucagon/aP2. The biological activity may be glucagon/aP2 binding to GCGR, detection of GCGR-mediated intracellular signal transduction; or determination of an end-point physiological effect. Representative, but non-limiting, examples of GCGR biological activity upon agonistic stimulation by glucagon/aP2 include, but are not limited to, signaling and regulation of the processes discussed herein, e.g., inhibition of cyclic AMP formation, reduction in glycogenolysis, reduced expression of gluconeogenic enzymes including phosphoenolpyruvate carboxykinase (PEPCK), fructose-1,6-bisphosphate, and glucose-6-phosphatase, inactivation of glycogen phosphorylase, and increased glycogen synthase activity. In one embodiment, the compound is a small molecule, a ligand, an antibody, antigen binding agent, or antibody fragment that binds to aP2, glucagon, and or glucagon/aP2 and neutralizes the ability of glucagon/aP2 to agonize GCGR. Methods of measuring biological effect of GCGR stimulation are known in the art and non-limiting examples of assays to detect GCGR biological activity are further exemplified in the Example below, and include, assays relating to reduced expression of gluconeogenic enzymes including phosphoenolpyruvate carboxykinase (PEPCK), fructose-1,6-bisphosphate (FBPase-1), and glucose-6-phosphatase (G-6-Pase) (See Example 1; FIGS. 1A and 1B; FIGS. 2A, 2C, and 2D), reduced hepatic glucose production (See Example 1; FIG. 1C), decreased glycogenolysis (See Example 1; FIG. 1D), and inhibition of cyclic AMP formation (See Example 1; FIGS. 1E and 1F).

In one non-limiting illustrative example, the cellular assay can be performed with varying concentrations of glucagon/aP2, GCGR, and/or compound to confirm, for example, the efficacy of the ability of the compound to interfere in glucagon/aP2 agonizing GCGR. For example, as described in the fifth aspect of the invention above, the first cellular assay of the fifth aspect of the present invention may be conducted as follows. 1 equivalent of the compound of interest is added to a solution of cells expressing GCGR in the presence of 1 equivalent of aP2 and 1 equivalent of glucagon. The activity of GCGR is then measured using any method described herein or known in the art. In a typical embodiment, the concentration of the compound of interest is equal to or higher than that of aP2 and glucagon in the cellular assay. In one embodiment, the concentration of the compound of interest is about 1, 2, 3, 4, 5, 10, 15, or 20 equivalents and the concentration of aP2 and glucagon is about 1 equivalent. Methods to measure the activity of GCGR in the presence of the compound of interest include those described herein and discussed in the paper by Thomas D. Pollard “A Guide to Simple and Informative Binding Assays”, MBOC; 2010; vol. 21 no. 23 4061.

In one non-limiting illustrative example, the second cellular assay of the fifth aspect of the present invention may be conducted as follows. 1 equivalent of the compound of interest is added to a solution of cells expressing GCGR in the presence of 20 equivalents of aP2 and 20 equivalents of glucagon. The activity of GCGR is then measured using any method described herein or known in the art. In a typical embodiment, the concentration of the compound of interest is less than that of aP2 and glucagon in the cellular assay (i.e. aP2 and glucagon are saturated with respect to the compound of interest). In one embodiment, the concentration of the aP2 and glucagon is about 5, 10, 15, 20, 25, 30, 35, or 40 equivalents and the concentration of the compound of interest is 1 equivalent.

In one embodiment, the equivalency of the compound of interest to glucagon and aP2 is not known and instead a concentration of compound is used.

In one non-limiting illustrative example, the cellular assays of the sixth aspect of the present invention may be conducted as follows. 0.5 equivalent of the compound of interest is added to a solution of cells expressing GCGR in the presence of 1 equivalent of aP2 and 1 equivalent of glucagon. The activity of GCGR is then measured using any method described herein or known in the art. Then the assay is serially repeated using 1 equivalent of the compound of interest, followed by 1.5 equivalents, 2 equivalents, etc. In one embodiment, the above procedure is conducted via serial dilution, starting with the highest concentration of compound and diluting it repeatedly to attain the lowest concentration. Methods to measure the activity of GCGR in the presence of the compound of interest include those described herein and discussed in the paper by Thomas D. Pollard “A Guide to Simple and Informative Binding Assays”, MBOC; 2010; vol. 21 no. 23 4061. In one embodiment, the concentration of the compound of interest is varied logarithmically for example 100 equivalents, 10 equivalents, 1 equivalent, and 0.1 equivalents of compound. In another embodiment, the equivalents of compound is not known and instead a concentration of the compound is varied, for example 100 mM, 10 mM, 1 mM, 100 nM, 10 nM, and 1 nM could be the concentrations used.

Methods for selecting a compound, for example an antibody, that selectively bind to glucagon/aP2 over aP2 alone are also provided. Methods for identifying preferably binding antibodies are generally known in the field. In one embodiment, provided herein is a method of identifying an antibody that selectively binds glucagon/aP2 over aP2 generally comprising administering to a non-human animal, for example a rabbit, mouse, rat, or goat, a heterologous glucagon/aP2 protein complex, for example human glucagon/aP2, in order to raise antibodies against the heterologous glucagon/aP2 in complex, isolating said antibodies, subjecting said antibodies to one or more binding assays measuring the binding affinity to glucagon/aP2 and aP2 alone, for example a competitive binding assay, wherein antibodies that preferably bind glucagon/aP2 over aP2 are isolated for use to neutralize glucagon/aP2 agonism of GCGR. For example, antibodies to glucagon/aP2 can be raised using hybridomas accomplished by standard procedures well known to those skilled in the field of immunology. Preferred methods for determining mAb specificity and affinity by competitive inhibition can be found in Harlow, et al., Antibodies: A Laboratory Manual, Cold Spring Harbor Laboratory Press, Cold Spring Harbor, N.Y., 1988), Colligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference.

Fusion partner cell lines and methods for fusing and selecting hybridomas and screening for mAbs are well known in the art. The glucagon/aP2 specific mAb can be produced in large quantities by injecting hybridoma or transfectoma cells secreting the antibody into the peritoneal cavity of mice and, after appropriate time, harvesting the ascites fluid which contains a high titer of the mAb, and isolating the mAb therefrom. For such in vivo production of the mAb with a non-murine hybridoma (e.g., rat or human), hybridoma cells are preferably grown in irradiated or athymic nude mice. Alternatively, the antibodies can be produced by culturing hybridoma or transfectoma cells in vitro and isolating secreted mAb from the cell culture medium or recombinantly, in eukaryotic or prokaryotic cells.

It should be noted that the methods for identifying the compounds above are considered to be illustrative and not restrictive.

aP2 and/or Glucagon-aP2 Complex Neutralizing Compounds

In one aspect of the invention, methods for modulating GCGR signaling are provided which include administering to a subject a compound that neutralizes the agonism of GCGR by glucagon/aP2 by inhibiting the formation of the glucagon/aP2 complex or the interaction of the glucagon/aP2 complex with GCGR by directly targeting glucagon/aP2, glucagon, or aP2, effectively neutralizing glucagon/aP2's ability to stimulate GCGR. In one embodiment, the compound an anti-aP2 and/or anti-glucagon/aP2 complex antibody, antibody fragment, or antigen binding agent. including, for example a monoclonal antibody, antibody fragment, or antigen binding agent. In one embodiment, the compound is a humanized monoclonal antibody or antigen binding agent. In one embodiment, the antibody, antibody fragment, or antigen binding agent preferentially binds to glucagon/aP2 over aP2 and glucagon. In one embodiment, the antibody, antibody fragment, or antigen binding agent preferentially binds to glucagon/aP2 over aP2 and glucagon. In one embodiment, the antibody, antibody fragment, or antigen binding agent does not bind to GCGR.

Methods of producing antibodies, antibody fragments, or antigen binding agents are known in the art. See, e.g., US2011/0129464. For example, polyclonal antibodies are preferably raised in animals by multiple subcutaneous (sc) or intraperitoneal (ip) injections of the relevant antigen and an adjuvant, for example, aP2, glucagon, or preferably glucagon in complex with aP2 (glucagon/aP2). It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl2, or R1N═C═NR, where R and R1 are different alkyl groups.

For example, animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567). In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et. al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (MA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy chain and light chain constant domains in place of the homologous murine sequences (U.S. Pat. No. 4,816,567; and Morrison, et al., Proc. Natl. Acad. Sci. USA, 81:6851(1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding. Various forms of the humanized antibody or affinity matured antibody are contemplated. For example, the humanized antibody or affinity matured antibody may be an antibody fragment, such as a Fab, which is optionally conjugated with one or more cytotoxic agent(s) in order to generate an immunoconjugate. Alternatively, the humanized antibody or affinity matured antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immunol., 7:33 (1993); and U.S. Pat. Nos. 5,591,669, 5,589,369 and 5,545,807.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S. and Chiswell, David J., Current Opinion in Structural Biology 3:564-571(1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894; and 5,587,458. The antibody fragment may also be a “linear antibody”, e.g., as described in U.S. Pat. No. 5,641,870 for example. Such linear antibody fragments may be monospecific or bispecific.

Techniques for generating antibodies have been described above. One may further select antibodies with certain biological characteristics, as desired, for example, preferential binding to the glucagon/aP2 complex over aP2 and/or glucagon/and/or GCGR.

To identify an antibody which inhibits glucagon/aP2 agonism of the GCGR receptor, the ability of the antibody to glucagon/aP2 ligand binding to cells expressing the GCGR may be determined. For example, cells naturally expressing, or transfected to express, GCGR receptors may be incubated with the antibody and then exposed to labeled glucagon/aP2. The ability of the anti-glucagon/aP2 antibody to block binding to GCGR may then be evaluated.

For example, inhibition of glucagon/aP2 binding to GCGR in hepatocytes by anti-glucagon/aP2 monoclonal antibodies may be performed using monolayer hepatocyte cultures on ice in a 24-well-plate format. Anti-glucagon/aP2 monoclonal antibodies may be added to each well and incubated for 30 minutes. ¹²⁵I-labeled glucagon or aP2 or glucagon/aP2 may then be added, and the incubation may be continued for 4 to 16 hours. Dose response curves may be prepared and an IC50 value may be calculated for the antibody of interest. In one embodiment, the antibody which blocks ligand activation of GCGR receptor will have an IC50 for inhibiting glucagon/aP2 binding to hepatocyte cells in this assay of about 50 nM or less, more preferably 10 nM or less. Where the antibody is an antibody fragment such as a Fab fragment, the IC50 for inhibiting glucagon/aP2 binding to GCGR on hepatocyte cells in this assay may, for example, be about 100 nM or less, more preferably 50 nM or less.

Alternatively, or additionally, the ability of the anti-glucagon/aP2 antibody to block glucagon/aP2 ligand-stimulated cAMP production through GCGR may be assessed. For example, cells endogenously expressing the GCGR or transfected to expressed GCGR may be incubated with the antibody and then assayed for glucagon/aP2 ligand-dependent cAMP activity.

In one embodiment, antibodies and fragments administered contain a light chain or light chain fragment having a variable region, wherein said variable region comprises one, two or three CDRs independently selected from Seq. ID No. 7, Seq. ID No. 8, and Seq. ID No. 9, Seq. ID No. 10, Seq. ID No. 11, Seq. ID No. 12 and Seq. ID No. 13. Alternatively, one or more of the disclosed and selected CDRs can be altered by substitution of one or more amino acids (for example, 1, 2, 3, 4, 5, 6, 7 or 8 amino acids) that do not adversely affect or that improve the properties of the antibody or antigen binding agent, as further described herein. In one embodiment, the selected CDR(s) is/are placed in a human immunoglobulin framework. In one embodiment, the human immunoglobulin framework is further modified or altered to maintain the binding affinity specificity of the grafted CDR region.

In one aspect of the present invention, an antibody or antigen binding agent is administered to a subject wherein the antibody comprises at least one, or more than one, of the CDR regions provided in Table 2.

TABLE 2
Anti-aP2/aP2-glucagon protein complex
Antibody Complementarity Determining Regions

| Protein | Seq. ID No. | SEQUENCE |
|---|---|---|
| CDRL1 | 7 | QASEDISRYLV |
| CDRL1 variant 1 | 22 | SVSSSISSSNLH |
| CDRL2 | 8 | KASTLAS |
| CDRL2 variant 1 | 23 | GTSNLAS |
| CDRL3 | 9 | QCTYGTYAGSFFYS |
| CDRL3 variant 1 | 10 | QATYGTYAGSFFYS |
| CDRL3 variant 2 | 11 | QQTYGTYAGSFFYS |
| CDRL3 variant 3 | 12 | QHTYGTYAGSFFYS |
| CDRL3 variant 4 | 13 | QQASHYPLT |
| CDRL3 variant 5 | 24 | QQWSHYPLT |
| CDRH1 | 14 | GFSLSTYYMS |
| CDRH1 variant 1 | 15 | GYTFTSNAIT |
| CDRH1 variant 2 | 25 | GYTFTSNWIT |
| CDRH2 | 16 | IIYPSGSTYCASWAKG |
| CDRH2 variant 1 | 17 | IIYPSGSTYSASWAKG |
| CDRH2 variant 2 | 18 | DISPGSGSTTNNEKFKS |
| CDRH2 variant 3 | 26 | DIYPGSGSTTNNEKFKS |
| CDRH3 | 19 | PDNDGTSGYLSGFGL |
| CDRH3 variant 1 | 20 | PDNEGTSGYLSGFGL |
| CDRH3 variant 2 | 21 | LRGFYDYFDF |
| CDRH3 variant 3 | 27 | LRGYYDYFDF |

In one embodiment, the glucagon/aP2 neutralizing antibody or antigen binding fragment is a monoclonal antibody or antigen binding fragment comprising a light chain wherein the variable domain comprises one, two, or three CDRs independently selected from CDRL1 (QASEDISRYLV) (Seq. ID No. 7), CDRL1 variant 1 (SVSSSISSSNLH) (Seq. ID No. 22), CDRL2 (KASTLAS) (Seq. ID No. 8), CDRL2 variant 1 (GTSNLAS) (Seq. ID No. 23), CDRL3 (QCTYGTYAGSFFYS) (Seq. ID. No. 9), CDRL3 variant 1 (QATYGTYAGSFFYS) (Seq. ID No. 10), CDRL3 variant 2 (QQTYGTYAGSFFYS) (Seq. ID No. 11), CDRL3 variant 3 (QHTYGTYAGSFFYS) (Seq. ID No. 12), CDRL3 variant 4 (QQASHYPLT) (Seq. ID No. 13), or CDRL3 variant 5 (QQWSHYPLT) (Seq. ID No. 24). In one embodiment, the antibody or antigen binding agent comprises a light chain variable region comprising CDRL1 (Seq. ID No. 7), CDRL2 (Seq. ID No. 8), and CDRL3 (Seq. ID No. 9). In one embodiment, the antibody or antigen binding agent comprises a light chain variable region comprising CDRL1 (Seq. ID No. 7), CDRL2 (Seq. ID No. 8), and CDRL3 variant 1 (Seq. ID No. 10). In one embodiment, the antibody or antigen binding agent comprises a light chain variable region comprising CDRL1 (Seq. ID No. 7), CDRL2 (Seq. ID No. 8), and CDRL3 variant 2 (Seq. ID No. 11). In one embodiment, the antibody or antigen binding agent comprises a light chain variable region comprising CDRL1 (Seq. ID No. 7), CDRL2 (Seq. ID No. 8), and CDRL3 variant 3 (Seq. ID No. 12).

In one embodiment, the antibody or antigen binding agent comprises a light chain variable region comprising CDRL3 variant 4 (Seq. ID No. 13), wherein the antibody has a Kd of about ≥10⁻⁷M. In one embodiment, the antibody or antigen binding agent comprises a light chain variable region comprising CDRL1 variant 1 (Seq. ID No. 22), CDRL2 variant 1 (Seq. ID No. 23), and CDRL3 variant 4 (Seq. ID No. 13). In one embodiment, the antibody or antigen binding agent comprises a light chain variable region comprising CDRL3 variant 4 (Seq. ID No. 13) and a heavy chain variable region comprising CDHR1 variant 1 (GYTFTSNAIT) (Seq. ID No. 15), CDRH2 variant 2 (DISPGSGSTTNNEKFKS) (Seq. ID No. 18), and, in one embodiment, CDRH3 variant 2 (LRGFYDYFDF) (Seq. ID No. 21).

In one embodiment, the antibody or antigen binding agent comprises one, two, or three CDRs selected from CDRL1 (Seq. ID No. 7), CDRL2 (Seq. ID No. 8), CDRL3 (Seq. ID No. 9), CDRL3 variant 1 (Seq. ID No. 10), CDRL3 variant 2 (Seq. ID No. 11), CDRL3 variant 3 (Seq. ID No. 12), and CDRL3 variant 4 (Seq. ID No. 13), and has a Kd of about ≥10⁻⁷ M. In one embodiment, the CDR sequences identified above are grafted into a human immunoglobulin framework. In one embodiment, the human immunoglobulin framework is further modified or altered, for example within the Vernier zone, to maintain the binding affinity specificity of the grafted CDR region.

In one embodiment, the antibody or antigen binding agent comprises a light chain wherein the variable domain comprises one, two, or three CDRs independently selected from an amino acid sequence that is at least 80%, 85%, 90%, or 95% homologous with CDRL1 (Seq. ID No. 7), CDRL2 (Seq. ID No. 8), CDRL3 (Seq. ID No. 9), CDRL3 variant 1 (Seq. ID No. 10), CDRL3 variant 2 (Seq. ID No. 11), CDRL3 variant 3 (Seq. ID No. 12), or CDRL3 variant 4 (Seq. ID No. 13). In one embodiment, the antibody or antigen binding agent has a Kd of about ≥10⁻⁷ M. In one embodiment, the CDR sequences identified above are grafted into a human immunoglobulin framework. In one embodiment, the human immunoglobulin framework is further modified or altered, for example within the Vernier zone, to maintain the binding affinity specificity of the grafted CDR region. In one embodiment, the antibody or antigen binding agent comprises a light chain wherein the variable domain comprises one, two, or three CDRs independently selected from an amino acid sequence that has one or more (for example, 1, 2, 3, or 4) amino acid substitutions, additions, or deletions as compared with CDRL1 (Seq. ID No. 7), CDRL2 (Seq. ID No. 8), CDRL3 (Seq. ID No. 9), CDRL3 variant 1 (Seq. ID No. 10), CDRL3 variant 2 (Seq. ID No. 11), CDRL3 variant 3 (Seq. ID No. 12), or CDRL3 variant 4 (Seq. ID No. 13).

In one embodiment, the antibody or antigen binding agent comprises a light chain wherein the variable domain comprises one, two, or three CDRs selected from CDRL1 (Seq. ID No. 7), CDRL2 (Seq. ID No. 8), CDRL3 (Seq. ID No. 9), CDRL3 variant 1 (Seq. ID No. 10), CDRL3 variant 2 (Seq. ID No. 11), CDRL3 variant 3 (Seq. ID No. 12), or CDRL3 variant 4 (Seq. ID No. 13), and one, two, or three CDRs selected from CDRH1 (GFSLSTYYMS) (Seq. ID NO. 14), CDRH1 variant 1 (Seq. ID No. 15), CDRH1 variant 2 (GYTFTSNWIT) (Seq. ID No. 25), CDRH2 (IIYPSGSTYCASWAKG) (Seq. ID No. 16), CDRH2 variant 1 (IIYPSGSTYSASWAKG) (Seq. ID No. 17), CDRH2 variant 2 (Seq. ID No. 18), CDRH2 variant 3 (DIYPGSGSTTNNEKFKS) (Seq. ID No. 26), CDHR3 (PDNDGTSGYLSGFGL) (Seq. ID No. 19), CDRH3 variant 1 (PDNEGTSGYLSGFGL) (Seq. ID No. 20), CDRH3 variant 2 (Seq. ID No. 21), or CDRH3 variant 3 (LRGYYDYFDFW) (Seq. ID No. 27). In one embodiment, the antibody or antigen binding agent comprises a heavy chain variable region comprising CDRH1 variant 1 (Seq. ID No. 15), CDRH2 variant 2 (Seq. ID No. 18), and CDRH3 variant 3 (Seq. ID No. 27). In one embodiment, the antibody or antigen binding agent comprises a heavy chain variable region comprising CDRH1 variant 1 (Seq. ID No. 15), CDRH2 variant 2 (Seq. ID No. 18), and CDRH3 variant 2 (Seq. ID No. 21). In one embodiment, the antibody or antigen binding agent has a Kd of about ≥10⁻⁷ M. In one embodiment, the CDR sequences identified above are grafted into a human immunoglobulin framework. In one embodiment, the human immunoglobulin framework is further modified or altered, for example within the Vernier zone, to maintain the binding affinity specificity of the grafted CDR region.

In one embodiment, the antibody or antigen binding agent comprises one, two, or three CDRs selected from CDRH1 (Seq. ID NO. 14), CDRH1 variant 1 (Seq. ID No. 15), CDRH2 (Seq. ID No. 16), CDRH2 variant 1 (Seq. ID No. 17), CDRH2 variant 2 (Seq. ID No. 18), CDRH3 (Seq. ID No. 19), CDRH3 variant 1 (Seq. ID No. 20), or CDRH3 variant 2 (Seq. ID No. 21), and has a KD of about ≥10⁻⁷M. In one embodiment, the antibody or antigen binding agent comprises CDRs CDRH1 (Seq. ID No. 14), CDRH2 (Seq. ID No. 16), and CDRH3 (Seq. ID No. 19). In one embodiment, the antibody or antigen binding agent comprises CDRs CDRH1 (Seq. ID No. 14), CDRH2 variant 1 (Seq. ID No. 17), and CDHR3 variant 1 (Seq. ID No. 20). In one embodiment, the antibody comprises CDRs CDRH1 variant 1 (Seq. ID No. 15) and CDRH2 variant 2 (Seq. ID No. 18). In one embodiment, the antibody comprises CDRs CDRH1 variant 1 (Seq. ID No. 15), and CDRH2 variant 2 (Seq. ID No. 18), and CDRH3 variant 2 (Seq. ID No. 21). In one embodiment, the CDR sequences identified above are grafted into a human immunoglobulin framework. In one embodiment, the human immunoglobulin framework is further modified or altered, for example within the Vernier zone, to maintain the binding affinity specificity of the grafted CDR region. In one embodiment, the antibody or antigen binding agent comprises one, two, or three CDRs selected from an amino acid sequence that has one or more (for example, 1, 2, 3, or 4) amino acid substitutions, additions, or deletions as compared to CDRH1 (Seq. ID NO. 14), CDRH1 variant 1 (Seq. ID No. 15), CDRH2 (Seq. ID No. 16), CDRH2 variant 1 (Seq. ID No. 17), CDRH2 variant 2 (Seq. ID No. 18), CDRH3 (Seq. ID No. 19), CDRH3 variant 1 (Seq. ID No. 20), or CDRH3 variant 2 (Seq. ID No. 21).

In one embodiment, the antibody or antigen binding agent comprises a heavy chain wherein the variable domain comprises one, two, or three CDRs selected from an amino acid sequence that is at least 80%, 85%, 90%, or 95% homologous with CDRH1 (Seq. ID No. 14), CDRH1 variant 1 (Seq. ID No. 15), CDRH2 (Seq. ID No. 16), CDRH2 variant 1 (Seq. ID No. 17), CDRH2 variant 2 (Seq. ID No. 18), CDRH3 (Seq. ID No. 19), CDRH3 variant 1 (Seq. ID No. 20), or CDRH3 variant 2 (Seq. ID No. 21). In one embodiment, the antibody or antigen binding agent has a Kd of about ≥10⁻⁷ M. In one embodiment, the CDR sequences identified above are grafted into a human immunoglobulin framework. In one embodiment, the human immunoglobulin framework is further modified or altered, for example within the Vernier zone, to maintain the binding affinity specificity of the grafted CDR region.

CDRs can be altered or modified to provide for improved binding affinity, minimize loss of binding affinity when grafted into a different backbone, or to decrease unwanted interactions between the CDR and the hybrid framework as described further below.

In one aspect of the present invention, the antibodies and fragments for administration are humanized.

Construction of CDR-grafted antibodies is generally described in European Patent Application EP-A-0239400, which discloses a process in which the CDRs of a mouse monoclonal antibody are grafted onto the framework regions of the variable domains of a human immunoglobulin by site directed mutagenesis using long oligonucleotides, and is incorporated herein. The CDRs determine the antigen binding specificity of antibodies and are relatively short peptide sequences carried on the framework regions of the variable domains.

The human variable heavy and light chain germline subfamily classification can be derived from the Kabat germline subgroup designations: VH1, VH2, VH3, VH4, VH5, VH6 or VH7 for a particular VH sequence and JH1, JH2, JH3, JH4, JH5, and JH6 for a for a particular variable heavy joining group for framework 4; VK1, VK2, VK3, VK4, VK5 or VK6 for a particular VL kappa sequence for framework 1, 2, and 3, and JK1, JK2, JK3, JK4, or JK5 for a particular kappa joining group for framework 4; or VL1, VL2, VL3, VL4, VL5, VL6, VL7, VL8, VL9, or VL10 for a particular VL lambda sequence for framework 1, 2, and 3, and JL1, JL2, JL3, or JL7 for a particular lambda joining group for framework 4.

The general framework of the light chain comprises the structures selected from FR1-CDRL1-FR2-CDRL2-FR3-CDRL3-FR4 and FR1-CDRL1-FR2-CDRL2-FR3-CDRL3-FR4-CL, and variations thereof, wherein the CDR regions are selected from at least one variable light chain CDR selected from Seq. ID Nos. 7-13, the framework regions are selected from either an immunoglobulin kappa light chain variable framework region, or an immunoglobulin lambda light chain variable framework region, and an immunoglobulin light chain constant region from either a kappa light chain constant region when the framework region is a kappa light chain variable framework region, or a lambda light chain constant region when the framework region is a lambda light chain variable framework region.

In one embodiment, the general framework of the heavy chain regions contemplated herein comprises the structures selected from FR1-CDRH1-FR2-CDRH2-FR3-CDRH3-FR4, FR1-CDRH1-FR2-CDRH2-FR3-CDRH3-FR4-CH1, FR1-CDRH1-FR2-CDRH2-FR3-CDRH3-FR4-CH1-Hinge-CH2 for IgG, IgD, and IgA immunoglobulin classes and FR1-CDRH1-FR2-CDRH2-FR3-CDRH3-FR4-CH1-CH2 for IgM and IgE immunoglobulin classes, FR1-CDRH1-FR2-CDRH2-FR3-CDRH3-FR4-CH1-Hinge-CH2-CH3 for IgG, IgD, and IgA immunoglobulin classes, FR1-CDRH1-FR2-CDRH2-FR3-CDRH3-FR4-CH1-CH2-CH3 for IgM and IgE immunoglobulin classes, and FR1-CDRH1-FR2-CDRH2-FR3-CDRH3-FR4-CH1-CH2-CH3-CH4 for IgM and IgE immunoglobulin classes, and variations thereof, wherein the CDR regions are selected from at least one variable heavy chain CDR selected from Seq. ID Nos. 14-21, and the framework regions are selected from heavy chain variable framework regions, and the heavy chain constant regions. IgA and IgM classes can further comprise a joining polypeptide that serves to link two monomer units of IgM or IgA together, respectively. In the case of IgM, the J chain-joined dimer is a nucleating unit for the IgM pentamer, and in the case of IgA it induces larger polymers.

The constant region domains of the antibody molecule for administration, if present, may be selected having regard to the proposed function of the antibody molecule, and in particular the effector functions which may be required. For example, the constant region domains may be human IgA, IgD, IgE, IgG or IgM domains. In particular embodiments, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required.

In one embodiment, the antibody administered comprises a variable light chain selected from Seq. ID. Nos. 28-36 or 37-40 (Table 3 below). In one embodiment, the antibody administered comprises a variable heavy chain selected from Seq. ID. Nos. 41-51 (Table 4 below). In one embodiment, the antibody administered comprises a variable light chain selected from Seq. ID. Nos. 28-36 or 487-490 and/or a variable heavy chain selected from Seq. ID. Nos. 41-51 or an antibody sequence which is 80% similar or more identical to Seq. ID. Nos. 28-36, 37-40 and/or a variable heavy chain selected from Seq. ID. Nos. 41-51, for example 85%, 90%, 91%, 92%, 93%, 94%, 95% 96%, 97%, 98% or 99% over part or whole of the relevant sequence, for example a variable domain sequence, a CDR sequence or a variable domain sequence excluding the CDRs.

TABLE 3
Sequences of Humanized Anti-aP2/glucagon/aP2 protein complex
Light Chain Regions

| | Seq. ID | |
|---|---|---|
| Protein | No. | Sequence |
| Rabbit Ab 909 VL- | 28 | DVVMTQTPASVSEPVGGTVTIKCQASEDISRYLVWYQ |
| region | | QKPGQPPKRLIYKASTLASGVPSRFKGSGSGTDFTLTIS |
| | | DLECDDAATYYCQCTYGTYAGSFFYSFGGGTEVVVE |
| 909 gL1 VL-region | 29 | DVVMTQSPSSLSASVGDRVTITCQASEDISRYLVWYQ |
| | | QKPGKAPKRLIYKASTLASGVPSRFKGSGSGTDFTLTIS |
| | | SLQPEDFATYYCQCTYGTYAGSFFYSFGGGTKVEIK |
| 909 gL1 VL + CL- | 30 | DVVMTQSPSSLSASVGDRVTITCQASEDISRYLVWYQ |
| region | | QKPGKAPKRLIYKASTLASGVPSRFKGSGSGTDFTLTIS |
| | | SLQPEDFATYYCQCTYGTYAGSFFYSFGGGTKVEIKRT |
| | | VAAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| 909 gL10 VL- | 31 | DVVMTQSPSSLSASVGDRVTITCQASEDISRYLVWYQ |
| region | | QKPGKAPKRLIYKASTLASGVPSRFKGSGSGTDFTLTIS |
| | | SLQPEDFATYYCQATYGTYAGSFFYSFGGGTKVEIK |
| 909 gL10 VL + | 32 | DVVMTQSPSSLSASVGDRVTITCQASEDISRYLVWYQ |
| CL-region | | QKPGKAPKRLIYKASTLASGVPSRFKGSGSGTDFTLTIS |
| | | SLQPEDFATYYCQATYGTYAGSFFYSFGGGTKVEIKRT |
| | | VAAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| 909 gL54 VL- | 33 | DVVMTQSPSSLSASVGDRVTITCQASEDISRYLVWYQ |
| region | | QKPGKAPKRLIYKASTLASGVPSRFKGSGSGTDFTLTIS |
| | | SLQPEDFATYYCQQTYGTYAGSFFYSFGGGTKVEIK |
| 909 gL54 VL + | 34 | DVVMTQSPSSLSASVGDRVTITCQASEDISRYLVWYQ |
| CL-region | | QKPGKAPKRLIYKASTLASGVPSRFKGSGSGTDFTLTIS |
| | | SLQPEDFATYYCQQTYGTYAGSFFYSFGGGTKVEIKRT |
| | | VAAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| 909 gL55 VL- | 35 | DVVMTQSPSSLSASVGDRVTITCQASEDISRYLVWYQ |
| region | | QKPGKAPKRLIYKASTLASGVPSRFKGSGSGTDFTLTIS |
| | | SLQPEDFATYYCQHTYGTYAGSFFYSFGGGTKVEIK |
| 909 gL55 VL + | 36 | DVVMTQSPSSLSASVGDRVTITCQASEDISRYLVWYQ |
| CL-region | | QKPGKAPKRLIYKASTLASGVPSRFKGSGSGTDFTLTIS |
| | | SLQPEDFATYYCQHTYGTYAGSFFYSFGGGTKVEIKRT |
| | | VAAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| 909 gL13 VL- | 37 | DIQMTQSPSSLSASVGDRVTITCQASEDISRYLVWYQQ |
| region | | KPGKAPKRLIYKASTLASGVPSRFSGSGSGTEFTLTISSL |
| | | QPEDFATYYCQATYGTYAGSFFYSFGGGTKVEIK |
| 909 gL13 VL + | 38 | DIQMTQSPSSLSASVGDRVTITCQASEDISRYLVWYQQ |
| CL-region | | KPGKAPKRLIYKASTLASGVPSRFSGSGSGTEFTLTISSL |
| | | QPEDFATYYCQATYGTYAGSFFYSFGGGTKVEIKRTV |
| | | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| 909 gL50 VL- | 39 | DVVMTQSPSSLSASVGDRVTITCQASEDISRYLVWYQ |
| region | | QKPGKAPKRLIYKASTLASGVPSRFKGSGSGTDFTLTIS |
| | | SLQPEDFATYYAQATYGTYAGSFFYSFGGGTKVEIK |
| 909 gL50 VL + | 40 | DVVMTQSPSSLSASVGDRVTITCQASEDISRYLVWYQ |
| CL-region | | QKPGKAPKRLIYKASTLASGVPSRFKGSGSGTDFTLTIS |
| | | SLQPEDFATYYAQATYGTYAGSFFYSFGGGTKVEIKRT |
| | | VAAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |

TABLE 4
Sequences of Humanized aP2/glucagon/aP2 protein complex
Heavy Chain Regions

| | Seq. ID | |
|---|---|---|
| Protein | No. | Sequence |
| Rabbit Ab 909 VH | 41 | QSVEESGGRLVTPGTPLTLTCTVSGFSLSTYYMSWVRQ |
| region | | APGKGLEWIGIIYPSGSTYCASWAKGRFTISKASTTVDL |
| | | KITSPTTEDTATYFCARPDNDGTSGYLSGFGLWGQGTL |
| | | VTVSS |
| 909gH1 VH region | 42 | EVQLQESGPGLVKPSGTLSLTCTVSGFSLSTYYMSWVR |
| | | QPPGKGLEWIGIIYPSGSTYCASWAKGRFTISKASTTVD |
| | | LKLSSVTAADTATYFCARPDNDGTSGYLSGFGLWGQG |
| | | TLVTVSS |
| 909gH1 IgG4 VH + | 43 | EVQLQESGPGLVKPSGTLSLTCTVSGFSLSTYYMSWVR |
| human γ-4P | | QPPGKGLEWIGIIYPSGSTYCASWAKGRFTISKASTTVD |
| constant | | LKLSSVTAADTATYFCARPDNDGTSGYLSGFGLWGQG |
| | | TLVTVSSASTKGPSVFPLAPCSRSTSESTAALGCLVKD |
| | | YFPEPVTVSWNSGALTSGVHTFPAVLQSSGLYSLSSVV |
| | | TVPSSSLGTKTYTCNVDHKPSNTKVDKRVESKYGPPCP |
| | | PCPAPEFLGGPSVFLFPPKPKDTLMISRTPEVTCVVVDV |
| | | SQEDPEVQFNWYVDGVEVHNAKTKPREEQFNSTYRV |
| | | VSVLTVLHQDWLNGKEYKCKVSNKGLPSSIEKTISKA |
| | | KGQPREPQVYTLPPSQEEMTKNQVSLTCLVKGFYPSDI |
| | | AVEWESNGQPENNYKTTPPVLDSDGSFFLYSRLTVDK |
| | | SRWQEGNVFSCSVMHEALHNHYTQK SLSLSLGK |
| 909gH14 VH | 44 | EVQLQESGPG |
| region | | LVKPSGTLSLTCAVSGFSLSTYYMSWVRQP |
| | | PGKGLEWIGIIYPSGSTYCASWAKGRFTISKASTKNTV |
| | | DLKLSSVTAADTATYFCARPDNDGTSGYLSGFGLWGQ |
| | | GTLVTVSS |
| 909gH14 IgG4 VH + | 45 | EVQLQESGPGLVKPSGTLSLTCAVSGFSLSTYYMSWV |
| human γ-4P | | RQPPGKGLEWIGIIYPSGSTYCASWAKGRFTISKASTKN |
| constant | | TVDLKLSSVTAADTATYFCARPDNDGTSGYLSGFGLW |
| | | GQGTLVTVSSASTKGPSVFPLAPCSRSTSESTAALGCL |
| | | VKDYFPEPVTVSWNSGALTSGVHTFPAVLQSSGLYSLS |
| | | SVVTVPSSSLGTKTYTCNVDHKPSNTKVDKRVESKYG |
| | | PPCPPCPAPEFLGGPSVFLFPPKPKDTLMISRTPEVTCV |
| | | VVDVSQEDPEVQFNWYVDGVEVHNAKTKPREEQFNS |
| | | TYRVVSVLTVLHQDWLNGKEYKCKVSNKGLPSSIEKT |
| | | ISKAKGQPREPQVYTLPPSQEEMTKNQVSLTCLVKGFY |
| | | PSDIAVEWESNGQPENNYKTTPPVLDSDGSFFLYSRLT |
| | | VDKSRWQEGNVFSCSVMHEALHNHYTQKSLSLSLGK |
| 909 gH15 VH | 46 | EVQLQESGPGLVKPSGTLSLTCTVSGFSLSTYYMSWVR |
| region | | QPPGKGLEWIGIIYPSGSTYSASWAKGRFTISKASTKNT |
| | | VDLKLSSVTAADTATYFCARPDNEGTSGYLSGFGLWG |
| | | QGTLVTVSS |
| 909gH15 IgG4 VH + | 47 | EVQLQESGPGLVKPSGTLSLTCTVSGFSLSTYYMSWVR |
| human γ-4P | | QPPGKGLEWIGIIYPSGSTYSASWAKGRFTISKASTKNT |
| constant | | VDLKLSSVTAADTATYFCARPDNEGTSGYLSGFGLWG |
| | | QGTLVTVSSASTKGPSVFPLAPCSRSTSESTAALGCLV |
| | | KDYFPEPVTVSWNSGALTSGVHTFPAVLQSSGLYSLSS |
| | | VVTVPSSSLGTKTYTCNVDHKPSNTKVDKRVESKYGP |
| | | PCPPCPAPEFLGGPSVFLFPPKPKDTLMISRTPEVTCVV |
| | | VDVSQEDPEVQFNWYVDGVEVHNAKTKPREEQFNST |
| | | YRVVSVLTVLHQDWLNGKEYKCKVSNKGLPSSIEKTI |
| | | SKAKGQPREPQVYTLPPSQEEMTKNQVSLTCLVKGFY |
| | | PSDIAVEWESNGQPENNYKTTPPVLDSDGSFFLYSRLT |
| | | VDKSRWQEGNVFSCSVMHEALHNHYTQKSLSLSLGK |
| 909 gH61 VH | 48 | EVQLQESGPGLVKPSGTLSLTCAVSGFSLSTYYMSWV |
| region | | RQPPGKGLEWIGIIYPSGSTYCASWAKGRVTISKDSSK |
| | | NQVSLKLSSVTAADTAVYYCARPDNDGTSGYLSGFGL |
| | | WGQGTLVTVSS |
| 909gH61 IgG4 VH + | 49 | EVQLQESGPGLVKPSGTLSLTCAVSGFSLSTYYMSWV |
| human γ-4P | | RQPPGKGLEWIGIIYPSGSTYCASWAKGRVTISKDSSK |
| constant | | NQVSLKLSSVTAADTAVYYCARPDNDGTSGYLSGFGL |
| | | WGQGTLVTVSSASTKGPSVFPLAPCSRSTSESTAALGC |
| | | LVKDYFPEPVTVSWNSGALTSGVHTFPAVLQSSGLYSL |
| | | SSVVTVPSSSLGTKTYTCNVDHKPSNTKVDKRVESKY |
| | | GPPCPPCPAPEFLGGPSVFLFPPKPKDTLMISRTPEVTC |
| | | VVVDVSQEDPEVQFNWYVDGVEVHNAKTKPREEQFN |
| | | STYRVVSVLTVLHQDWLNGKEYKCKVSNKGLPSSIEK |
| | | TISKAKGQPREPQVYTLPPSQEEMTKNQVSLTCLVKGF |
| | | YPSDIAVEWESNGQPENNYKTTPPVLDSDGSFFLYSRL |
| | | TVDKSRWQEGNVFSCSVMHEALHNHYTQKSLSLSLG |
| | | K |
| 909 gH62 VH | 50 | EVQLQESGPGLVKPSGTLSLTCAVSGFSLSTYYMSWV |
| region | | RQPPGKGLEWIGIIYPSGSTYSASWAKGRVTISKDSSKN |
| | | QVSLKLSSVTAADTAVYYCARPDNEGTSGYLSGFGLW |
| | | GQGTLVTVSS |
| 909gH62 IgG4 VH + | 51 | EVQLQESGPGLVKPSGTLSLTCAVSGFSLSTYYMSWV |
| human γ-4P | | RQPPGKGLEWIGIIYPSGSTYSASWAKGRVTISKDSSKN |
| constant | | QVSLKLSSVTAADTAVYYCARPDNEGTSGYLSGFGLW |
| | | GQGTLVTVSSASTKGPSVFPLAPCSRSTSESTAALGCL |
| | | VKDYFPEPVTVSWNSGALTSGVHTFPAVLQSSGLYSLS |
| | | SVVTVPSSSLGTKTYTCNVDHKPSNTKVDKRVESKYG |
| | | PPCPPCPAPEFLGGPSVFLFPPKPKDTLMISRTPEVTCV |
| | | VVDVSQEDPEVQFNWYVDGVEVHNAKTKPREEQFNS |
| | | TYRVVSVLTVLHQDWLNGKEYKCKVSNKGLPSSIEKT |
| | | ISKAKGQPREPQVYTLPPSQEEMTKNQVSLTCLVKGFY |
| | | PSDIAVEWESNGQPENNYKTTPPVLDSDGSFFLYSRLT |
| | | VDKSRWQEGNVFSCSVMHEALHNHYTQKSLSLSLGK |

In one embodiment, the antibody molecule administered is a Fab, Fab′, or F(ab′)2 antibody fragment comprising a light chain variable region selected from Seq. ID Nos. 29, 31, 37, 38, 33, or 35, and a heavy chain variable region selected from Seq. ID Nos. 42, 44, 46, 48, or 50.

In one embodiment, the antibody molecule of the present disclosure is a full length IgG1 antibody comprising the variable regions shown in Seq. ID Nos. 29, 31, 37, 38, 33, or 35 for the light chain and Seq. ID Nos. 42, 44, 46, 48, or 50 for the heavy chain.

In one embodiment, the antibody molecule of the present disclosure is a full length IgG4 antibody comprising the variable regions shown in Seq. ID Nos. 29, 31, 37, 38, 33, or 35 for the light chain and Seq. ID Nos. 42, 44, 46, 48, or 50 for the heavy chain.

In one embodiment, the antibody molecule of the present disclosure is a full length IgG4P antibody comprising the variable regions shown in Seq. ID Nos. 29, 31, 37, 38, 33, or 35 for the light chain and Seq. ID Nos. 42, 44, 46, 48, or 50 for the heavy chain.

In one embodiment, the fusion protein administered comprises two domain antibodies, for example as a variable heavy (VH) and variable light (VL) pairing, optionally linked by a disulphide bond.

The antibody fragment administered may include Fab, Fab′, F(ab′)2, scFv, diabody, scFAb, dFv, single domain light chain antibodies, dsFv, a peptide comprising CDR, and the like.

Methods of Treating Disorders Associated with GCGR Agonism

Methods are provided for neutralizing GCGR agonism by the glucagon/aP2 complex (glucagon/aP2) within the liver, where it regulates hepatic glucose output, on the kidney, and on islet β-cells, reflecting its role in gluconeogenesis, intestinal smooth muscle, brain, and adipose tissue. Because of the prominent role glucagon/aP2 complex plays in inducing hepatic glucose production by agonizing GCGR, neutralizing, either fully or partially, GCGR agonism has the ability to modulate the severity of underlying conditions and disorders associated with dysregulated GCGR stimulation. In one embodiment, a compound which interferes with the formation of the glucagon/aP2 complex or the ability of the glucagon/aP2 complex to agonize GCGR is administered to a subject having an underlying condition or disorder associated with excessive or dysregulated GCGR stimulation. In one embodiment, a monoclonal antibody as described herein is used to neutralize glucagon/aP2's ability to agonize GCGR.

An antibody, antigen-binding agent or antibody-binding fragment that targets the glucagon/aP2 protein complex, including anti-glucagon/aP2 protein complex humanized antibody, antigen-binding agent or antibody-binding fragments, is useful in treating metabolic disorders involving dysregulated glucagon signaling resulting in chronic elevated blood glucose levels, including, but not limited to, diabetes (Type I and Type II), obesity, and nonalcoholic fatty liver disease (NAFLD), nonalcoholic steatohepatitis (NASH), metabolic disorders, cardiovascular disease, atherosclerosis, fibrosis, cirrhosis, hepatocellular carcinoma, insulin resistance, dyslipidemia, hyperglycemia, hyperglucanemia, hyperinsulinemia. For example, the anti-glucagon/aP2 protein complex antibody, antigen-binding agent or antibody-binding fragments described herein are capable of binding to secreted aP2 and/or glucagon/aP2 protein complex at a low-binding affinity, which, when administered to a host in need thereof, neutralizes glucagon receptor activity and provides lower fasting blood glucose levels, improved systemic glucose metabolism, increased systemic insulin sensitivity, reduced fat mass, liver steatosis, improved serum lipid profiles, and/or reduced atherogenic plaque formation in a host.

In one aspect of the present invention, a method is provided for treating a disease or disorder caused by dysregulated glucagon activity resulting in an aberrant level of excess glucose in the blood of a host by administering an effective amount of an antibody, antigen-binding agent or antibody-binding fragment that targets the glucagon/aP2 protein complex. In one embodiment, the disorder is a metabolic disorder. In one embodiment, the disorder is diabetes. In one embodiment, the disorder is Type I diabetes. In one embodiment, the disorder is Type II diabetes. In one embodiment, the disorder is hyperglycemia. In one embodiment, the disorder is obesity. In one embodiment, the disorder is dyslipidemia. In one embodiment, the disorder is nonalcoholic fatty liver disease (NAFLD). In one embodiment, the disorder is nonalcoholic steatoheptatis (NASH).

Diabetes

Diabetes mellitus is the most common metabolic disease worldwide. Every day, 1700 new cases of diabetes are diagnosed in the United States, and at least one-third of the 16 million Americans with diabetes are unaware of it. Diabetes is the leading cause of blindness, renal failure, and lower limb amputations in adults and is a major risk factor for cardiovascular disease and stroke.

In one aspect of the present invention, a method is provided for treating diabetes by administering to a host an effective amount of an antibody, antigen-binding agent or antibody-binding fragment that targets glucagon/aP2 protein complex. In one embodiment, the disorder is Type I diabetes. In one embodiment, the disorder is Type II diabetes.

Type I diabetes results from autoimmune destruction of pancreatic beta cells causing insulin deficiency. Type II or non-insulin-dependent diabetes mellitus (NIDDM) accounts for >90% of cases and is characterized by a resistance to insulin action on glucose uptake in peripheral tissues, especially skeletal muscle and adipocytes, impaired insulin action to inhibit hepatic glucose production, and misregulated insulin secretion.

In one embodiment of the present invention, provided herein is a method of treating Type I diabetes in a host by administering to the host an effective amount of an antibody, antigen-binding agent or antibody-binding fragment that targets the glucagon/aP2 protein complex in combination or alteration with insulin. In one embodiment of the present invention, provided herein is a method of treating Type I diabetes in a host by administering to the host an effective amount of antibody, antigen-binding agent or antibody-binding fragment that targets the glucagon/aP2 protein complex in combination or alteration with a synthetic insulin analog.

Some people who have Type II diabetes can achieve their target blood sugar levels with diet and exercise alone, but many also need diabetes medications or insulin therapy. In one embodiment of the present invention, provided herein is a method of treating Type II diabetes in a host by administering to the host an effective amount of an antibody, antigen-binding agent or antibody-binding fragment that targets the glucagon/aP2 protein complex. In one embodiment, provided herein is a method of treating a disease or condition associated with diabetes by administering to a host an effective amount of an antibody, antigen-binding agent or antibody-binding fragment that targets the glucagon/aP2 protein complex. Diseases and conditions associated with diabetes mellitus can include, but are not restricted to, hyperglycemia, hyperinsulinaemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis and ulcerative colitis. Furthermore, diseases and conditions associated with diabetes mellitus comprise, but are not restricted to: coronary heart disease, hypertension, angina pectoris, myocardial infarction, stroke, skin and connective tissue disorders, foot ulcerations, metabolic acidosis, arthritis, osteoporosis and in particular conditions of impaired glucose tolerance.

Body Weight Disorders

In one embodiment of the present invention, a method is provided for treating obesity due to dysregulated glucagon activity in a host by administering an effective amount of an antibody, antigen-binding agent or antibody-binding fragment that targets the glucagon/aP2 protein complex. Obesity represents the most prevalent of body weight disorders, affecting an estimated 30 to 50% of the middle-aged population in the western world.

In one embodiment of the present invention, a method is provided for treating obesity in a host by administering an effective amount of an antibody, antigen-binding agent or antibody-binding fragment that targets the glucagon/aP2 protein complex. In one embodiment, a method is provided for reducing or inhibiting weight gain caused by dysregulated glucagon activity in a host by administering an effective amount of an antibody, antigen-binding agent or antibody-binding fragment that targets the glucagon/aP2 protein complex.

Nonalcoholic Fatty Liver Disease (NAFLD)

There is a need for compositions and methods for the treatment and prevention of the development of fatty liver and conditions stemming from fatty liver, such as nonalcoholic steatohepatitis (NASH), liver inflammation, cirrhosis and liver failure caused by dysregulation of glucagon and chronic hyperglycemia. In one embodiment of the present invention, a method is provided for treating NAFLD in a host by administering an effective amount of an antibody, antigen-binding agent or antibody-binding fragment that targets the glucagon/aP2 protein complex.

Nonalcoholic Steatohepatitis (NASH)

Nonalcoholic steatohepatitis (NASH), which is an advanced form of nonalcoholic fatty liver disease (NAFLD), refers to the accumulation of hepatic steatosis not due to excess alcohol consumption. NASH is a liver disease characterized by inflammation of the liver with concurrent fat accumulation. NASH is also frequently found in people with diabetes and obesity and is related to metabolic syndrome. NASH is the progressive form of the relatively benign non-alcoholic fatty liver disease, for it can slowly worsen causing fibrosis accumulation in the liver, which leads to cirrhosis (reviewed in Smith et al., (2011), Crit. Rev. Clin. Lab. Sci., 48(3):97-113). Currently, no approved therapies for NASH exist.

In one embodiment of the present invention, a method is provided for treating NASH in a host by administering an effective amount of an antibody, antigen-binding agent or antibody-binding fragment that targets the glucagon/aP2 protein complex.

Glucagonoma and Necrolytic Migratory Erythema

A glucagonoma is a rare tumor of the alpha cells of the pancreas that results in the overproduction of the hormone glucagon. The primary physiological effect of glucagonoma is an overproduction of the peptide hormone glucagon. Necrolytic migratory erythema (NME) is a classical symptom observed in patients with glucagonoma and is the presenting problem in 70% of cases (van Beek et al., (November 2004). “The glucagonoma syndrome and necrolytic migratory erythema: a clinical review”. Eur. J. Endocrinol. 151 (5): 531-7). Associated NME is characterized by the spread of erythematous blisters and swelling across areas subject to greater friction and pressure, including the lower abdomen, buttocks, perineum, and groin.

In one embodiment of the present invention, a method is provided to treat glucagonoma and/or necrolytic migratory erythemain (NME) a host by administering an effective amount of an antibody, antigen-binding agent or antibody-binding fragment that targets the glucagon/aP2 protein complex. In one embodiment, the antibody or antibody binding agent contains a light chain or light chain fragment having a variable region, wherein said variable region comprises one, two, or three complementarity determining regions (CDRs) independently selected from Seq. ID No. 7, Seq. ID No. 8, and Seq. ID No. 9. In another embodiment, the antibody or antigen binding agent administered to a subject comprises a light chain or light chain fragment having a variable region, wherein said variable region comprises one, two, or three CDRs independently selected from Seq. ID No. 10, Seq. ID No. 11, Seq. ID No. 12, Seq. ID No. 13, Seq. ID No. 22, Seq. ID No. 23, or Seq. ID No. 24. In still another embodiment, the antibody or antibody binding agent administered comprises a light chain or light chain fragment having a variable region, wherein said variable region comprises one, two, or three CDRs independently selected from Seq. ID No. 7, Seq. ID No. 8 and Seq. ID No. 9, Seq. ID No. 10, Seq. ID No. 11, Seq. ID No. 12, Seq. ID No. 13, Seq. ID No. 22, Seq. ID No. 23, or Seq. ID No. 24. In one embodiment, the antibody or antibody binding agent administered to a subject comprises a light chain or light chain fragment having a variable region, wherein said variable region comprises Seq. ID No. 7, Seq. ID. No. 8, and at least one CDR selected from Seq. ID. No. 9, Seq. ID No. 10, Seq. ID No. 11, Seq. ID No. 12, Seq. ID No. 13, Seq. ID No. 22, Seq. ID No. 23, or Seq. ID No. 24. Alternatively, one or more of the disclosed and selected CDRs can be altered by substitution of one or more amino acids that do not adversely affect or that improve the properties of the antibody or antigen binding agent, as further described herein. In one embodiment, the selected CDR(s) is/are placed in a human immunoglobulin framework. In one embodiment, the human immunoglobulin framework is further modified or altered to maintain the binding affinity specificity of the grafted CDR region. In one embodiment, the antibody or antibody binding agent administered has a KD for human aP2 of ≥10−7 M.

In one embodiment, the antibody or antibody binding agent administered to a subject includes at least one CDR selected from Seq. ID Nos. 7-13 or Seq. ID Nos. 22-24, and at least one CDR selected from CDRH1 (Seq. ID NO. 14), CDRH1 variant 1 (Seq. ID No. 15), CDRH1 variant 2 (Seq. ID No. 25), CDRH2 (Seq. ID No. 16), CDRH2 variant 1 (Seq. ID No. 17), CDRH2 variant 2 (Seq. ID No. 18), CDRH2 variant 3 (Seq. ID No. 26), CDHR3 (Seq. ID No. 19), CDHR3 variant 1 (Seq. ID No. 20), CDRH3 variant 2 (Seq. ID No. 21), or CDRH3 variant 3 (Seq. ID No. 27), wherein the CDR sequences are grafted into a human immunoglobulin framework. In one embodiment, the human immunoglobulin framework is further modified or altered to maintain the binding affinity specificity of the grafted CDR region.

In certain embodiments, the antibody or antigen binding agent administered includes at least the light chain variable sequence 909 gL1 (Seq. ID No. 29), the light chain sequence 909 gL1 VL+CL (Seq. ID No. 30), the light chain variable sequence 909 gL10 (Seq. ID No. 31), the light chain sequence 909 gL10 VL+CL (Seq. ID No. 32), the light chain variable sequence 909 gL13 (Seq. ID No. 37), the light chain sequence 909 gL13 VL+CL (Seq. ID No. 39), the light chain variable sequence 909 gL50 (Seq. ID No. 38), the light chain sequence 909 gL50 VL+CL (Seq. ID No. 40), the light chain variable sequence 909 gL54 (Seq. ID No. 33), the light chain sequence 909 gL54 VL+CL (Seq. ID No. 34), the light chain variable sequence 909 gL55 (Seq. ID No. 35) or the light chain sequence 909 gL55 VL+CL (Seq. ID No. 36).

In other embodiments, the antibody or antigen binding agent administered includes a light chain variable sequence selected from 909 gL1 (Seq. ID No. 29), 909 gL10 (Seq. ID No. 31), 909 gL13 (Seq. ID No. 37), 909 gL50 (Seq. ID No. 38), 909 gL54 (Seq. ID No. 33), or 909 gL55 (Seq. ID No. 35), and a heavy chain variable sequence selected from 909 gH1 (Seq. ID No. 42), 909 gH14 (Seq. ID No. 44), 909 gH15 (Seq. ID No. 46), 909 gH61 (Seq. ID No. 48), or 909 gH62 (Seq. ID No. 50). For example, the antibody or antigen binding agent can include at least the light chain variable sequence 909 gL1 (Seq. ID No. 29) and the heavy chain variable sequence 909 gH1 (Seq. ID. No. 42).

Metabolic Disorders

In one aspect of the present invention, a method is provided for treating metabolic disorder in a host mediated by dysregulated glucagon activity by administering an effective amount of an antibody, antigen-binding agent or antibody-binding fragment that targets the glucagon/aP2 protein complex. A metabolic disorder includes a disorder, disease, or condition, which is caused or characterized by an abnormal metabolism (i.e., the chemical changes in living cells by which energy is provided for vital processes and activities) in a subject. Metabolic disorders include diseases, disorders, or conditions associated with hyperglycemia. Metabolic disorders can detrimentally affect cellular functions such as cellular proliferation, growth, differentiation, or migration, cellular regulation of homeostasis, inter- or intra-cellular communication; tissue function, such as liver function, renal function, or adipocyte function; systemic responses in an organism, such as hormonal responses (e.g., glucagon response). Examples of metabolic disorders include obesity, diabetes, hyperphagia, endocrine abnormalities, triglyceride storage disease, Bardet-Biedl syndrome, Laurence-Moon syndrome, Prader-Labhart-Willi syndrome, and disorders of lipid metabolism.

Methods of Attenuating the Severity of a Glucagon Receptor Mediated Disorder

A method of preventing or treating a disease or disorder caused by a dysregulation in glucagon activity resulting in an aberrant level of excess glucose in the blood of a host, typically a human, is provided by administering to the host a therapeutically effective amount of an antibody, antigen-binding agent or antibody-binding fragment that targets the glucagon/aP2 protein complex. The antibody, antigen-binding agent or antibody-binding fragment is administered at a dose sufficient to inhibit or reduce the biological activity of glucagon/aP2 protein complex either partially or fully.

In one aspect, a method of preventing or attenuating the severity of an glucagon-mediated disorder in a host is provided by administering an effective amount of an antibody, antigen-binding agent or antibody-binding fragment that targets the glucagon/aP2 protein complex, resulting in the reduction or attenuation of the biological activity of glucagon, and a reduction in the associated physiological effects of dysregulated glucagon, for example, a reduction in fasting blood glucose levels, fat mass levels, hepatic glucose production, fat cell lipolysis, hyperinsulinemia, and/or liver steatosis. In one embodiment, the attenuation of the biological activity of glucagon results in an increase in insulin sensitivity, glucose metabolism, and/or the prevention of islet β-cell death, dysfunction, or loss.

In other aspects of the present invention, methods are providing for:

reducing fasting blood glucose levels;

reducing fat mass levels;

reducing hepatic glucose production;

reducing fat cell lipolysis;

reducing hyperinsulinemia;

reducing liver steatosis;

increasing glucose metabolism;

increasing insulin sensitivity;

preventing β-cell death, dysfunction, or loss; and/or

determining circulating secreted aP2 levels in a host;

comprising administering an effective amount of an antibody, antigen-binding agent or antibody-binding fragment that targets the glucagon/aP2 protein complex to a host, typically a human, in need thereof.

### Combination Therapies

A compound capable of interfering with glucagon/aP2 agonism of GCGR can be used to treat an underlying disorder mediated through excessive GCGR agonism. In one embodiment, the compound is administered to a subject in need thereof in combination or alternation with an additional active ingredient.

In some embodiments, provided herein are methods utilizing combination therapy wherein a compound capable of interfering with glucagon/aP2 agonism of GCGR are administered to a subject with another therapeutic agent. Examples of additional therapeutic agents that may be administered in combination with a compound of the present invention, and either administered separately or in the same pharmaceutical composition, include, but are not limited to:

(a) anti-diabetic agents such as (1) PPARγ agonists such as glitazones (e.g. ciglitazone; darglitazone; englitazone; isaglitazone (MCC-555); pioglitazone (ACTOS); rosiglitazone (AVANDIA); troglitazone; rivoglitazone, BRL49653; CLX-0921; 5-BTZD, GW-0207, LG-100641, R483, and LY-300512, and the like and compounds disclosed in WO97/10813, 97/27857, 97/28115, 97/28137, 97/27847, 03/000685, and 03/027112 and SPPARMS (selective PPAR gamma modulators) such as T131 (Amgen), FK614 (Fujisawa), netoglitazone, and metaglidasen; (2) biguanides such as buformin; metformin; and phenformin, and the like; (3) protein tyrosine phosphatase-1B (PTP-1B) inhibitors such as ISIS 113715, A-401674, A-364504, IDD-3, IDD 2846, KP-40046, KR61639, MC52445, MC52453, C7, OC-060062, OC-86839, 0C29796, TTP-277BC1, and those agents disclosed in WO 04/041799, 04/050646, 02/26707, 02/26743, 04/092146, 03/048140, 04/089918, 03/002569, 04/065387, 04/127570, and US 2004/167183; (4) sulfonylureas such as acetohexamide; chlorpropamide; diabinese; glibenclamide; glipizide; glyburide; glimepiride; gliclazide; glipentide; gliquidone; glisolamide; tolazamide; and tolbutamide, and the like; (5) meglitinides such as repaglinide, metiglinide (GLUFAST) and nateglinide, and the like; (6) alpha glucoside hydrolase inhibitors such as acarbose; adiposine; camiglibose; emiglitate; miglitol; voglibose; pradimicin-Q; salbostatin; CKD-711; MDL-25,637; MDL-73,945; and MOR 14, and the like; (7) alpha-amylase inhibitors such as tendamistat, trestatin, and Al-3688, and the like; (8) insulin secreatagogues such as linogliride nateglinide, mitiglinide (GLUFAST), ID1101 A-4166, and the like; (9) fatty acid oxidation inhibitors, such as clomoxir, and etomoxir, and the like; (10) A2 antagonists, such as midaglizole; isaglidole; deriglidole; idazoxan; earoxan; and fluparoxan, and the like; (11) insulin or insulin mimetics, such as biota, LP-100, novarapid, insulin detemir, insulin lispro, insulin glargine, inulin degludec, insulin zinc suspension (lente and ultralente); Lys-Pro insulin, GLP-1 (17-36), GLP-1 (73-7) (insulintropin); GLP-1 (7-36)-NH2) exenatide/Exendin-4, Exenatide LAR, Linaglutide, AVE0010, CJC 1131, BIM51077, CS 872, TH0318, BAY-694326, GP010, ALBUGON (GLP-1 fused to albumin), HGX-007 (Epac agonist), S-23521, and compounds disclosed in WO 04/022004, WO 04/37859, and the like; (12) non-thiazolidinediones such as JT-501, and farglitazar (GW-2570/GI-262579), and the like; (13) PPARa/γ dual agonists such as AVE 0847, CLX-0940, GW-1536, GW1929, GW-2433, KRP-297, L-796449, LBM 642, LR-90, LY510919, MK-0767, ONO 5129, SB 219994, TAK-559, TAK-654, 677954 (GlaxoSmithkline), E-3030 (Eisai), LY510929 (Lilly), AK109 (Asahi), DRF2655 (Dr. Reddy), DRF8351 (Dr. Reddy), MC3002 (Maxocore), TY51501 (ToaEiyo), aleglitazar, farglitazar, naveglitazar, muraglitazar, peliglitazar, tesaglitazar (GALIDA), reglitazar (JT-501), chiglitazar, and those disclosed in WO 99/16758, WO 99/19313, WO 99/20614, WO 99/38850, WO 00/23415, WO 00/23417, WO 00/23445, WO 00/50414, WO 01/00579, WO 01/79150, WO 02/062799, WO 03/033481, WO 03/033450, WO 03/033453; and (14), insulin, insulin mimetics and other insulin sensitizing drugs; (15) VPAC2 receptor agonists; (16) GLK modulators, such as PSN105, RO 281675, RO 274375 and those disclosed in WO 03/015774, WO 03/000262, WO 03/055482, WO 04/046139, WO 04/045614, WO 04/063179, WO 04/063194, WO 04/050645, and the like; (17) retinoid modulators such as those disclosed in WO 03/000249; (18) GSK 3beta/GSK 3 inhibitors such as 4-[2-(2-bromophenyl)-4-(4-fluorophenyl-1H-imidazol-5-yl]pyridine, CT21022, CT20026, CT-98023, SB-216763, SB410111, SB-675236, CP-70949, XD4241 and those compounds disclosed in WO 03/037869, 03/03877, 03/037891, 03/024447, 05/000192, 05/019218 and the like; (19) glycogen phosphorylase (HGLPa) inhibitors, such as AVE 5688, PSN 357, GPi-879, those disclosed in WO 03/037864, WO 03/091213, WO 04/092158, WO 05/013975, WO 05/013981, US 2004/0220229, and JP 2004-196702, and the like; (20) ATP consumption promotors such as those disclosed in WO 03/007990; (21) fixed combinations of PPAR γ agonists and metformin such as AVANDAMET; (22) PPAR pan agonists such as GSK 677954; (23) GPR40 (G-protein coupled receptor 40) also called SNORF 55 such as BG 700, and those disclosed in WO 04/041266, 04/022551, 03/099793; (24) GPR119 (G-protein coupled receptor 119, also called RUP3; SNORF 25) such as RUP3, HGPRBMY26, PFI 007, SNORF 25; (25) adenosine receptor 2B antagonists such as ATL-618, AT1-802, E3080, and the like; (26) carnitine palmitoyl transferase inhibitors such as ST 1327, and ST 1326, and the like; (27) Fructose 1,6-bisphospohatase inhibitors such as CS-917, MB7803, and the like; (28) glucagon antagonists such as AT77077, BAY 694326, GW 4123X, NN2501, and those disclosed in WO 03/064404, WO 05/00781, US 2004/0209928, US 2004/029943, and the like; (30) glucose-6-phosphate inhibitors; (31) phosphoenolpyruvate carboxykinase (PEPCK) inhibitors; (32) pyruvate dehydrogenase kinase (PDK) activators; (33) RXR agonists such as MC1036, CS00018, JNJ 10166806, and those disclosed in WO 04/089916, U.S. Pat. No. 6,759,546, and the like; (34) SGLT inhibitors such as AVE 2268, KGT 1251, T1095/RWJ 394718; (35) BLX-1002; (36) alpha glucosidase inhibitors; (37) glucagon receptor agonists; (38) glucokinase activators; 39) GIP-1; 40) insulin secretagogues; 41) GPR-40 agonists, such as TAK-875, 5[4-[[(1R)-446-(3-hydroxy-3-methylbutoxy)-2-methylpyridine-3-yl]-2,3-dihydro-1H-indene-1-yl]oxy]phenyl] isothiazole-3-ol 1-oxide, 5-(4-((3-(2,6-dimethyl-4-(3-(methyl sulfonyl)propoxy)-phenyl)phenyl)-methoxy)phenyl)i so, 5-(4-((3-(2-methyl-6-(3-hydroxypropoxyl)pyridine-3-yl)-2-methylphenyl)methoxy)phenyl)isothiazole-3-ol 1-oxide, and 5-[4-[ [3-[4-(3-aminopropoxy)-2,6-dimethylphenyl]phenyl]methoxy]phenyl]isothiazole-3-ol 1-oxide), and those disclosed in WO 11/078371; 42) SGLT-2 inhibitors such as canagliflozin, dapagliflozin, tofogliflozin, empagliflozin, ipragliflozin, luseogliflozin (TS-071), ertugliflozin (PF-04971729), and remogliflozin; and 43) SGLT-1/SGLT-2 inhibitors, such as LX4211;

(b) anti-dyslipidemic agents such as (1) bile acid sequestrants such as, cholestyramine, colesevelem, colestipol, dialkylaminoalkyl derivatives of a cross-linked dextran; Colestid®; LoCholest®; and Questran®, and the like; (2) HMG-CoA reductase inhibitors such as atorvastatin, itavastatin, pitavastatin, fluvastatin, lovastatin, pravastatin, rivastatin, simvastatin, rosuvastatin (ZD-4522), and other statins, particularly simvastatin; (3) HMG-CoA synthase inhibitors; (4) cholesterol absorption inhibitors such as FMVP4 (Forbes Medi-Tech), KT6-971 (Kotobuki Pharmaceutical), FM-VA12 (Forbes Medi-Tech), FM-VP-24 (Forbes Medi-Tech), stanol esters, beta-sitosterol, sterol glycosides such as tiqueside; and azetidinones such as ezetimibe, and those disclosed in WO 04/005247 and the like; (5) acyl coenzyme A-cholesterol acyl transferase (ACAT) inhibitors such as avasimibe, eflucimibe, pactimibe (KY505), SMP 797 (Sumitomo), SM32504 (Sumitomo), and those disclosed in WO 03/091216, and the like; (6) CETP inhibitors such as anacetrapib, JTT 705 (Japan Tobacco), torcetrapib, CP 532,632, BAY63-2149 (Bayer), SC 591, SC 795, and the like; (7) squalene synthetase inhibitors; (8) anti-oxidants such as probucol, and the like; (9) PPARa agonists such as beclofibrate, bezafibrate, ciprofibrate, clofibrate, etofibrate, fenofibrate, gemcabene, and gemfibrozil, GW 7647, BM 170744 (Kowa), LY518674 (Lilly), GW590735 (GlaxoSmithkline), KRP-101 (Kyorin), DRF10945 (Dr. Reddy), NS-220/R1593 (Nippon Shinyaku/Roche, ST1929 (Sigma Tau) MC3001/MC3004 (MaxoCore Pharmaceuticals, gemcabene calcium, other fibric acid derivatives, such as Atromid®, Lopid® and Tricor®, and those disclosed in U.S. Pat. No. 6,548,538, and the like; (10) FXR receptor modulators such as GW 4064 (GlaxoSmithkline), SR 103912, QRX401, LN-6691 (Lion Bioscience), and those disclosed in WO 02/064125, WO 04/045511, and the like; (11) LXR receptor modulators such as GW 3965 (GlaxoSmithkline), T9013137, and XTC0179628 (X-Ceptor Therapeutics/Sanyo), and those disclosed in WO 03/031408, WO 03/063796, WO 04/072041, and the like; (12) lipoprotein synthesis inhibitors such as niacin; (13) renin angiotensin system inhibitors; (14) PPAR δ partial agonists, such as those disclosed in WO 03/024395; (15) bile acid reabsorption inhibitors, such as BARI 1453, SC435, PHA384640, 58921, AZD7706, and the like; and bile acid sequesterants such as colesevelam (WELCHOL/CHOLESTAGEL), colestipol, cholestyramine, and dialkylaminoalkyl derivatives of a cross-linked dextran, (16) PPAR agonists such as GW 501516 (Ligand, GSK), GW 590735, GW-0742 (GlaxoSmithkline), T659 (Amgen/Tularik), LY934 (Lilly), NNC610050 (Novo Nordisk) and those disclosed in WO97/28149, WO 01/79197, WO 02/14291, WO 02/46154, WO 02/46176, WO 02/076957, WO 03/016291, WO 03/033493, WO 03/035603, WO 03/072100, WO 03/097607, WO 04/005253, WO 04/007439, and JP10237049, and the like; (17) triglyceride synthesis inhibitors; (18) microsomal triglyceride transport (MTTP) inhibitors, such as implitapide, LAB687, JTT130 (Japan Tobacco), CP346086, and those disclosed in WO 03/072532, and the like; (19) transcription modulators; (20) squalene epoxidase inhibitors; (21) low density lipoprotein (LDL) receptor inducers; (22) platelet aggregation inhibitors; (23) 5-LO or FLAP inhibitors; and (24) niacin receptor agonists including HM74A receptor agonists; (25) PPAR modulators such as those disclosed in WO 01/25181, WO 01/79150, WO 02/79162, WO 02/081428, WO 03/016265, WO 03/033453; (26) niacin-bound chromium, as disclosed in WO 03/039535; (27) substituted acid derivatives disclosed in WO 03/040114; (28) infused HDL such as LUV/ETC-588 (Pfizer), APO-A1 Milano/ETC216 (Pfizer), ETC-642 (Pfizer), ISIS301012, D4F (Bruin Pharma), synthetic trimeric ApoAl, Bioral Apo A1 targeted to foam cells, and the like; (29) IBAT inhibitors such as BARI143/HMR145A/HMR1453 (Sanofi-Aventis, PHA384640E (Pfizer), 58921 (Shionogi) AZD7806 (AstraZeneca), AK105 (Asah Kasei), and the like; (30) Lp-PLA2 inhibitors such as SB480848 (GlaxoSmithkline), 659032 (GlaxoSmithkline), 677116 (GlaxoSmithkline), and the like; (31) other agents which affect lipic composition including ETC1001/ESP31015 (Pfizer), ESP-55016 (Pfizer), AGI1067 (AtheroGenics), AC3056 (Amylin), AZD4619 (AstrZeneca); and

(c) anti-hypertensive agents such as (1) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (4) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (5) neutral endopeptidase inhibitors such as omapatrilat, cadoxatril and ecadotril, fosidotril, sampatrilat, AVE7688, ER4030, and the like; (6) endothelin antagonists such as tezosentan, A308165, and YM62899, and the like; (7) vasodilators such as hydralazine, clonidine, minoxidil, and nicotinyl alcohol, nicotinic acid or salt thereof, and the like; (8) angiotensin II receptor antagonists such as candesartan, eprosartan, irbesartan, losartan, pratosartan, tasosartan, telmisartan, valsartan, and EXP-3137, FI6828K, and RNH6270, and the like; (9) α/β adrenergic blockers as nipradilol, arotinolol and amosulalol, and the like; (10) alpha 1 blockers, such as terazosin, urapidil, prazosin, bunazosin, trimazosin, doxazosin, naftopidil, indoramin, WHIP 164, and XEN010, and the like; (11) alpha 2 agonists such as lofexidine, tiamenidine, moxonidine, rilmenidine and guanobenz, and the like; (12) aldosterone inhibitors, and the like; (13) angiopoietin-2-binding agents such as those disclosed in WO 03/030833; and

(d) anti-obesity agents, such as (1) 5HT (serotonin) transporter inhibitors, such as paroxetine, fluoxetine, fenfluramine, fluvoxamine, sertraline, and imipramine, and those disclosed in WO 03/00663, as well as serotonin/noradrenaline re uptake inhibitors such as sibutramine (MERIDIA/REDUCTIL) and dopamine uptake inhibitor/Norepenephrine uptake inhibitors such as radafaxine hydrochloride, 353162 (GlaxoSmithkline), and the like; (2) NE (norepinephrine) transporter inhibitors, such as GW 320659, despiramine, talsupram, and nomifensine; (3) CB1 (cannabinoid-1 receptor) antagonist/inverse agonists, such as rimonabant (ACCOMPLIA Sanofi Synthelabo), SR-147778 (Sanofi Synthelabo), AVE1625 (Sanofi-Aventis), BAY 65-2520 (Bayer), SLV 319 (Solvay), SLV326 (Solvay), CP945598 (Pfizer), E-6776 (Esteve), 01691 (Organix), ORG14481 (Organon), VER24343 (Vernalis), NESS0327 (Univ of Sassari/Univ of Cagliari), and those disclosed in U.S. Pat. Nos. 4,973,587, 5,013,837, 5,081,122, 5,112,820, 5,292,736, 5,532,237, 5,624,941, 6,028,084, and 6,509,367; and WO 96/33159, WO97/29079, WO98/31227, WO 98/33765, WO98/37061, WO98/41519, WO98/43635, WO98/43636, WO99/02499, WO00/10967, WO00/10968, WO 01/09120, WO 01/58869, WO 01/64632, WO 01/64633, WO 01/64634, WO 01/70700, WO 01/96330, WO 02/076949, WO 03/006007, WO 03/007887, WO 03/020217, WO 03/026647, WO 03/026648, WO 03/027069, WO 03/027076, WO 03/027114, WO 03/037332, WO 03/040107, WO 04/096763, WO 04/111039, WO 04/111033, WO 04/111034, WO 04/111038, WO 04/013120, WO 05/000301, WO 05/016286, WO 05/066126 and EP-658546 and the like; (4) ghrelin agonists/antagonists, such as BVT81-97 (BioVitrum), RC1291 (Rejuvenon), SRD-04677 (Sumitomo), unacylated ghrelin (TheraTechnologies), and those disclosed in WO 01/87335, WO 02/08250, WO 05/012331, and the like; (5) H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, and those disclosed in WO 02/15905; and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO 03/024928 and WO 03/024929; (6) melanin-concentrating hormone 1 receptor (MCH1R) antagonists, such as T-226296 (Takeda), T71 (Takeda/Amgen), AMGN-608450, AMGN-503796 (Amgen), 856464 (GlaxoSmithkline), A224940 (Abbott), A798 (Abbott), ATC0175/AR224349 (Arena Pharmaceuticals), GW803430 (GlaxoSmithKline), NBI-1A (Neurocrine Biosciences), NGX-1 (Neurogen), SNP-7941 (Synaptic), SNAP9847 (Synaptic), T-226293 (Schering Plough), TPI-1361-17 (Saitama Medical School/University of California Irvine), and those disclosed WO 01/21169, WO 01/82925, WO 01/87834, WO 02/051809, WO 02/06245, WO 02/076929, WO 02/076947, WO 02/04433, WO 02/51809, WO 02/083134, WO 02/094799, WO 03/004027, WO 03/13574, WO 03/15769, WO 03/028641, WO 03/035624, WO 03/033476, WO 03/033480, WO 04/004611, WO 04/004726, WO 04/011438, WO 04/028459, WO 04/034702, WO 04/039764, WO 04/052848, WO 04/087680; and Japanese Patent Application Nos. JP 13226269, JP 1437059, JP2004315511, and the like; (7) MCH2R (melanin concentrating hormone 2R) agonist/antagonists; (8) NPY1 (neuropeptide Y Y1) antagonists, such as BMS205749, BIBP3226, J-115814, MO 3304, LY-357897, CP-671906, and GI-264879A; and those disclosed in U.S. Pat. No. 6,001,836; and WO 96/14307, WO 01/23387, WO 99/51600, WO 01/85690, WO 01/85098, WO 01/85173, and WO 01/89528; (9) NPY5 (neuropeptide Y Y5) antagonists, such as 152,804, 52367 (Shionogi), E-6999 (Esteve), GW-569180A, GW-594884A (GlaxoSmithkline), GW-587081X, GW-548118X; FR 235,208; FR226928, FR 240662, FR252384; 1229U91, GI-264879A, CGP71683A, C-75 (Fasgen) LY-377897, LY366377, PD-160170, SR-120562A, SR-120819A, S2367 (Shionogi), JCF-104, and H409/22; and those compounds disclosed in U.S. Pat. Nos. 6,140,354, 6,191,160, 6,258,837, 6,313,298, 6,326,375, 6,329,395, 6,335,345, 6,337,332, 6,329,395, and 6,340,683; and EP-01010691, EP-01044970, and FR252384; and PCT Publication Nos. WO 97/19682, WO 97/20820, WO 97/20821, WO 97/20822, WO 97/20823, WO 98/27063, WO 00/107409, WO 00/185714, WO 00/185730, WO 00/64880, WO 00/68197, WO 00/69849, WO 01/09120, WO 01/14376, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/20488, WO 02/22592, WO 02/48152, WO 02/49648, WO 02/051806, WO 02/094789, WO 03/009845, WO 03/014083, WO 03/022849, WO 03/028726, WO 05/014592, WO 05/01493; and Norman et al., J. Med. Chem. 43:4288-4312 (2000); (10) leptin, such as recombinant human leptin (PEG-OB, Hoffman La Roche) and recombinant methionyl human leptin (Amgen); (11) leptin derivatives, such as those disclosed in U.S. Pat. Nos. 5,552,524; 5,552,523; 5,552,522; 5,521,283; and WO 96/23513; WO 96/23514; WO 96/23515; WO 96/23516; WO 96/23517; WO 96/23518; WO 96/23519; and WO 96/23520; (12) opioid antagonists, such as nalmefene (Revex®), 3-methoxynaltrexone, naloxone, and naltrexone; and those disclosed in WO 00/21509; (13) orexin antagonists, such as SB-334867-A (GlaxoSmithkline); and those disclosed in WO 01/96302, 01/68609, 02/44172, 02/51232, 02/51838, 02/089800, 02/090355, 03/023561, 03/032991, 03/037847, 04/004733, 04/026866, 04/041791, 04/085403, and the like; (14) BRS3 (bombesin receptor subtype 3) agonists; (15) CCK-A (cholecystokinin-A) agonists, such as AR-R 15849, GI 181771, JMV-180, A-71378, A-71623, PD170292, PD 149164, SR146131, SR125180, butabindide, and those disclosed in U.S. Pat. No. 5,739,106; (16) CNTF (ciliary neurotrophic factors), such as GI-181771 (Glaxo-SmithKline); SR146131 (Sanofi Synthelabo); butabindide; and PD170,292, PD 149164 (Pfizer); (17) CNTF derivatives, such as axokine (Regeneron); and those disclosed in WO 94/09134, WO 98/22128, and WO 99/43813; (18) GHS (growth hormone secretagogue receptor) agonists, such as NN703, hexarelin, MK-0677, SM-130686, CP-424,391, L-692,429 and L-163,255, and those disclosed in U.S. Pat. No. 6,358,951, U.S. Patent Application Nos. 2002/049196 and 2002/022637; and WO 01/56592, and WO 02/32888; (19) 5HT2c (serotonin receptor 2c) agonists, such as APD3546/AR10A (Arena Pharmaceuticals), ATH88651 (Athersys), ATH88740 (Athersys), BVT933 (Biovitrum/GSK), DPCA37215 (BMS), IK264; LY448100 (Lilly), PNU 22394; WAY 470 (Wyeth), WAY629 (Wyeth), WAY161503 (Biovitrum), R-1065, VR1065 (Vernalis/Roche) YM 348; and those disclosed in U.S. Pat. No. 3,914,250; and PCT Publications 01/66548, 02/36596, 02/48124, 02/10169, 02/44152; 02/51844, 02/40456, 02/40457, 03/057698, 05/000849, and the like; (20) Mc3r (melanocortin 3 receptor) agonists; (21) Mc4r (melanocortin 4 receptor) agonists, such as CHIR86036 (Chiron), CHIR915 (Chiron); ME-10142 (Melacure), ME-10145 (Melacure), HS-131 (Melacure), NBI72432 (Neurocrine Biosciences), NNC 70-619 (Novo Nordisk), TTP2435 (Transtech) and those disclosed in PCT Publications WO 99/64002, 00/74679, 01/991752, 01/0125192, 01/52880, 01/74844, 01/70708, 01/70337, 01/91752, 01/010842, 02/059095, 02/059107, 02/059108, 02/059117, 02/062766, 02/069095, 02/12166, 02/11715, 02/12178, 02/15909, 02/38544, 02/068387, 02/068388, 02/067869, 02/081430, 03/06604, 03/007949, 03/009847, 03/009850, 03/013509, 03/031410, 03/094918, 04/028453, 04/048345, 04/050610, 04/075823, 04/083208, 04/089951, 05/000339, and EP 1460069, and US 2005049269, and JP2005042839, and the like; (22) monoamine reuptake inhibitors, such as sibutratmine (Meridia®/Reductil®) and salts thereof, and those compounds disclosed in U.S. Pat. Nos. 4,746,680, 4,806,570, and 5,436,272, and U.S. Patent Publication No. 2002/0006964, and WO 01/27068, and WO 01/62341; (23) serotonin reuptake inhibitors, such as dexfenfluramine, fluoxetine, and those in U.S. Pat. No. 6,365,633, and WO 01/27060, and WO 01/162341; (24) GLP-1 (glucagon-like peptide 1) agonists; (25) Topiramate (Topimax®); (26) phytopharm compound 57 (CP 644,673); (27) ACC2 (acetyl-CoA carboxylase-2) inhibitors; (28) (33 (beta adrenergic receptor 3) agonists, such as rafebergron/AD9677/TAK677 (Dainippon/Takeda), CL-316,243, SB 418790, BRL-37344, L-796568, BMS-196085, BRL-35135A, CGP12177A, BTA-243, GRC1087 (Glenmark Pharmaceuticals) GW 427353 (solabegron hydrochloride), Trecadrine, Zeneca D7114, N-5984 (Nisshin Kyorin), LY-377604 (Lilly), KT07924 (Kissei), SR 59119A, and those disclosed in U.S. Pat. Nos. 5,705,515, 5,451,677; and WO94/18161, WO95/29159, WO97/46556, WO98/04526 WO98/32753, WO 01/74782, WO 02/32897, WO 03/014113, WO 03/016276, WO 03/016307, WO 03/024948, WO 03/024953, WO 03/037881, WO 04/108674, and the like; (29) DGAT1 (diacylglycerol acyltransferase 1) inhibitors; (30) DGAT2 (diacylglycerol acyltransferase 2) inhibitors; (31) FAS (fatty acid synthase) inhibitors, such as Cerulenin and C75; (32) PDE (phosphodiesterase) inhibitors, such as theophylline, pentoxifylline, zaprinast, sildenafil, amrinone, milrinone, cilostamide, rolipram, and cilomilast, as well as those described in WO 03/037432, WO 03/037899; (33) thyroid hormone β agonists, such as KB-2611 (KaroBioBMS), and those disclosed in WO 02/15845; and Japanese Patent Application No. JP 2000256190; (34) UCP-1 (uncoupling protein 1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), and retinoic acid; and those disclosed in WO 99/00123; (35) acyl-estrogens, such as oleoyl-estrone, disclosed in del Mar-Grasa, M. et al., Obesity Research, 9:202-9 (2001); (36) glucocorticoid receptor antagonists, such as CP472555 (Pfizer), KB 3305, and those disclosed in WO 04/000869, WO 04/075864, and the like; (37) 11β HSD-1 (11-beta hydroxy steroid dehydrogenase type 1) inhibitors, such as LY-2523199, BVT 3498 (AMG 331), BVT 2733, 3-(1-adamantyl)-4-ethyl-5-(ethylthio)-4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][11]annulene, and those compounds disclosed in WO 01/90091, 01/90090, 01/90092, 02/072084, 04/011410, 04/033427, 04/041264, 04/027047, 04/056744, 04/065351, 04/089415, 04/037251, and the like; (38) SCD-1 (stearoyl-CoA desaturase-1) inhibitors; (39) dipeptidyl peptidase IV (DPP-4) inhibitors, such as isoleucine thiazolidide, valine pyrrolidide, sitagliptin (Januvia), omarigliptin, saxagliptin, alogliptin, linagliptin, NVP-DPP728, LAF237 (vildagliptin), P93/01, TSL 225, TMC-2A/2B/2C, FE 999011, P9310/1(364, VIP 0177, SDZ 274-444, GSK 823093, E 3024, SYR 322, TS021, SSR 162369, GRC 8200, K579, NN7201, CR 14023, PHX 1004, PHX 1149, PT-630, SK-0403; and the compounds disclosed in WO 02/083128, WO 02/062764, WO 02/14271, WO 03/000180, WO 03/000181, WO 03/000250, WO 03/002530, WO 03/002531, WO 03/002553, WO 03/002593, WO 03/004498, WO 03/004496, WO 03/005766, WO 03/017936, WO 03/024942, WO 03/024965, WO 03/033524, WO 03/055881, WO 03/057144, WO 03/037327, WO 04/041795, WO 04/071454, WO 04/0214870, WO 04/041273, WO 04/041820, WO 04/050658, WO 04/046106, WO 04/067509, WO 04/048532, WO 04/099185, WO 04/108730, WO 05/009956, WO 04/09806, WO 05/023762, US 2005/043292, and EP 1 258 476; (40) lipase inhibitors, such as tetrahydrolipstatin (orlistat/XENICAL), ATL962 (Alizyme/Takeda), GT389255 (Genzyme/Peptimmune) Triton WR1339, RHC80267, lipstatin, teasaponin, and diethylumbelliferyl phosphate, FL-386, WAY-121898, Bay-N-3176, valilactone, esteracin, ebelactone A, ebelactone B, and RHC 80267, and those disclosed in WO 01/77094, WO 04/111004, and U.S. Pat. Nos. 4,598,089, 4,452,813, 5,512,565, 5,391,571, 5,602,151, 4,405,644, 4,189,438, and 4,242,453, and the like; (41) fatty acid transporter inhibitors; (42) dicarboxylate transporter inhibitors; (43) glucose transporter inhibitors; and (44) phosphate transporter inhibitors; (45) anorectic bicyclic compounds such as 1426 (Aventis) and 1954 (Aventis), and the compounds disclosed in WO 00/18749, WO 01/32638, WO 01/62746, WO 01/62747, and WO 03/015769; (46) peptide YY and PYY agonists such as PYY336 (Nastech/Merck), AC162352 (IC Innovations/Curis/Amylin), TM30335/TM30338 (7TM Pharma), PYY336 (Emisphere Technologies), pegylated peptide YY3-36, those disclosed in WO 03/026591, 04/089279, and the like; (47) lipid metabolism modulators such as maslinic acid, erythrodiol, ursolic acid uvaol, betulinic acid, betulin, and the like and compounds disclosed in WO 03/011267; (48) transcription factor modulators such as those disclosed in WO 03/026576; (49) Mc5r (melanocortin 5 receptor) modulators, such as those disclosed in WO 97/19952, WO 00/15826, WO 00/15790, US 20030092041, and the like; (50) Brain derived neutotropic factor (BDNF), (51) Mc1r (melanocortin 1 receptor modulators such as LK-184 (Proctor & Gamble), and the like; (52) 5HT6 antagonists such as BVT74316 (BioVitrum), BVT5182c (BioVitrum), E-6795 (Esteve), E-6814 (Esteve), SB399885 (GlaxoSmithkline), SB271046 (GlaxoSmithkline), RO-046790 (Roche), and the like; (53) fatty acid transport protein 4 (FATP4); (54) acetyl-CoA carboxylase (ACC) inhibitors such as CP640186, CP610431, CP640188 (Pfizer); (55) C-terminal growth hormone fragments such as A0D9604 (Monash Univ/Metabolic Pharmaceuticals), and the like; (56) oxyntomodulin; (57) neuropeptide FF receptor antagonists such as those disclosed in WO 04/083218, and the like; (58) amylin agonists such as Symlin/pramlintide/AC137 (Amylin); (59) Hoodia and trichocaulon extracts; (60) BVT74713 and other gut lipid appetite suppressants; (61) dopamine agonists such as bupropion (WELLBUTRIN/GlaxoSmithkline); (62) zonisamide (ZONEGRAN/Dainippon/Elan), and the like; and

(e) anorectic agents suitable for use in combination with a compound of the present invention include, but are not limited to, aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; and pharmaceutically acceptable salts thereof. A particularly suitable class of anorectic agent are the halogenated amphetamine derivatives, including chlorphentermine, cloforex, clortermine, dexfenfluramine, fenfluramine, picilorex and sibutramine; and pharmaceutically acceptable salts thereof. Particular halogenated amphetamine derivatives of use in combination with a compound of the present invention include: fenfluramine and dexfenfluramine, and pharmaceutically acceptable salts thereof;

(f) CB1 (cannabinoid-1 receptor) antagonist/inverse agonists such as rimonabant (Acomplia; Sanofi), SR-147778 (Sanofi), SR-141 716 (Sanofi), BAY 65-2520 (Bayer), and SLV 319 (Solvay), and those disclosed in patent publications U.S. Pat. Nos. 4,973,587, 5,013,837, 5,081,122, 5,112,820, 5,292,736, 5,532,237, 5,624,941, U.S. Pat. Nos. 6,028,084, 6,509,367, 6,509,367, WO96/33159, WO97/29079, WO98/31227, WO98/33765, WO98/37061, WO98/41519, WO98/43635, WO98/43636, WO99/02499, WO00/10967, WO00/10968, WO01/09120, WO01/58869, WO01/64632, WO01/64633, WO01/64634, WO01/70700, WO01/96330, WO02/076949, WO03/006007, WO03/007887, WO03/020217, WO03/026647, WO03/026648, WO03/027069, WO03/027076, WO03/0271 14, WO03/037332, WO03/040107, WO03/086940, WO03/084943 and EP658546;

(g) CB1 receptor antagonists such as 1,5-diarylpyrazole analogues such as rimonabant (SR141716, Acomplia®, Bethin®, Monaslim®, Remonabent®, Riobant®, Slimona®, Rimoslim®, Zimulti® and Riomont®), surinabant (SR147778) and AM251; 3,4-diarylpyrazolines such as SLV-319 (ibipinabant); 4,5-diarylimidazoles; 1,5-diarylpyrrole-3-carboxamides, bicyclic derivatives of diaryl-pyrazole and imidazoles such as CP-945,598 (otenabant); methylsulfonamide azetidine derivatives; TM38837; beta-lactam cannabinoid modulators; benzofuran derivatives. CB1 receptor antagonists can include or exclude 1,5-diarylpyrazole analogues such as rimonabant (SR141716, Acomplia®, Bethin®, Monaslim®, Remonabent®, Riobant®, Slimona®, Rimoslim®, Zimulti® and Riomont®), surinabant (SR147778) and AM251; 3,4-diarylpyrazolines such as SLV-319 (ibipinabant); 4,5-diarylimidazoles; 1,5-diarylpyrrole-3-carboxamides, bicyclic derivatives of diaryl-pyrazole and imidazoles such as CP-945,598 (otenabant); methylsulfonamide azetidine derivatives; TM38837; beta-lactam cannabinoid modulators; and benzofuran derivatives.

### Pharmaceutical Compositions

A compound, for example, a small molecule, ligand, antibody, antigen-binding agent, or antibody-binding fragment that inhibits the glucagon/aP2 complex from agonizing GCGR useful in the treatment and/or prophylaxis of a pathological condition can be administered in an effective amount as a pharmaceutical composition comprising the compound in combination with one or more of a pharmaceutically acceptable excipient, diluent, or carrier. The composition will usually be supplied as part of a sterile, pharmaceutical composition that will normally include a pharmaceutically acceptable carrier. A pharmaceutical composition of the present invention may additionally comprise a pharmaceutically-acceptable excipient.

The compound disrupting the glucagon/aP2 complex agonism of GCGR may be the sole active ingredient in the pharmaceutical composition or may be accompanied by other active ingredients including other ingredients.

The pharmaceutical compositions suitably comprise a therapeutically effective amount of the compound that interrupts glucagon/aP2 complex agonism of GCGR. The term “therapeutically effective amount” as used herein refers to an amount of a therapeutic agent needed to inhibit glucagon/aP2 complex agonism of GCGR in such a way so as to treat, ameliorate, or prevent a targeted disease or condition, or to exhibit a detectable therapeutic or preventative effect mediated by GCGR. For any suitable compound, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

Accordingly, the disclosure provides pharmaceutical compositions comprising an effective amount of compound or pharmaceutically acceptable salt together with at least one pharmaceutically acceptable carrier for any of the uses described herein. The pharmaceutical composition may contain a compound or salt as the only active agent, or, in an alternative embodiment, the compound and at least one additional active agent.

The dosage administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired. In certain embodiments, the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 25, 50, 100, 200, 250, 300, 400, 500, 600, 700, or 750 mg of active compound, or its salt. As a non-limiting example, treatment of GCGR mediated pathologies in humans or animals can be provided as a daily dosage of anti-glucagon/aP2 monoclonal antibodies of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20, or any combination thereof, using single or divided doses of every 24, 12, 8, 6, 4, or 2 hours, or any combination thereof.

The pharmaceutical composition may also include a molar ratio of the active compound and an additional active agent. For example, the pharmaceutical composition may contain a molar ratio of about 0.5:1, about 1:1, about 2:1, about 3:1 or from about 1.5:1 to about 4:1 of an anti-inflammatory or immunosuppressing agent. Compounds disclosed herein may be administered orally, topically, parenterally, by inhalation or spray, sublingually, via implant, including ocular implant, transdermally, via buccal administration, rectally, as an ophthalmic solution, injection, including ocular injection, intraveneous, intra-aortal, intracranial, subdermal, intraperitioneal, subcutaneous, transnasal, sublingual, or rectal or by other means, in dosage unit formulations containing conventional pharmaceutically acceptable carriers.

The pharmaceutical composition may be formulated as any pharmaceutically useful form, e.g., as an aerosol, a cream, a gel, a pill, an injection or infusion solution, a capsule, a tablet, a syrup, a transdermal patch, a subcutaneous patch, a dry powder, an inhalation formulation, in a medical device, suppository, buccal, or sublingual formulation, parenteral formulation, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions/combinations can be formulated for oral administration. These compositions can contain any amount of active compound that achieves the desired result, for example between 0.1 and 99 weight % (wt. %) of the compound and usually at least about 5 wt. % of the compound. Some embodiments contain from about 25 wt. % to about 50 wt. % or from about 5 wt. % to about 75 wt. % of the compound.

Formulations suitable for rectal administration are typically presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. In one embodiment, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung. ose forms containing a predetermined amount of an active agent of the invention per dose.

Advantageously, the levels of glucagon/aP2 agonism of GCGR in vivo may be maintained at an appropriately reduced level by administration of sequential doses of a compound that interferes with the glucagon/aP2 agonism of GCGR according to the disclosure.

Compositions may be administered individually to a patient or may be administered in combination (e.g. simultaneously, sequentially, or separately) with other agents, drugs or hormones.

In one embodiment, compound is administered continuously, for example, the compound can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

### Examples

Dysregulated glucagon activity and elevated blood glucose levels resulting in chronic hyperglycemia has been implicated in the pathology of many metabolic diseases, such as diabetes.

### Example 1: Circulating aP2 Directly Interacts with Glucagon and is Required for Glucagon's Biological Activities

### Materials

All DNA and oligonucleotide synthesis was done by IDT DNA Technologies. L-169,047 (Glucagon Receptor Antagonist II) was purchased from Tocris Biosciences). All other reagents and chemicals were purchased from Sigma-Aldrich and used as received except where otherwise noted.

### Bio-Layer Interferometry (BLI) Measurements

The binding affinity of aP2 to biotin-glucagon was measured by a BLItz Bio-Layer Interferometry system (BLI, Fortébio Inc.) at 25° C. Bio-Layer Interferometry measures the change in the interference pattern of light as ligand in solution binds an immobilized target on a biosensor probe yielding an apparent Kd. Briefly, streptavidin BLItz Dip and Read™—kinetic biosensor probes (Fortébio Inc.) were loaded with 20 μg/mL of biotinylated glucagon in PBS buffer, washed in PBS buffer and baseline readings were taken for 30 seconds in PBS. Association phase readings for aP2 were performed for 200 seconds at 3.4 μM and 34 μM concentrations in PBS followed by dissociation phase in the same buffer for 300 seconds. The dissociation constant was obtained by global curve fitting of the responses to yield a kₒₙ value and a k_{off} value of which was then used to calculate Kdₐₚₚ. Background binding (apparent affinities) of aP2 interacting with mock-loaded probes was less than 2% of binding to albumin-loaded probes and background binding was subtracted from total binding.

### Scintillation Proximity Assays

aP2 was biotinylated using Pierce amine reactive biotinylation kit. ¹²⁵I labeled glucagon (Perkin Elmer) was incubated in Streptavidin coated flash plates (Perkin Elmer) in 5 mM MgCl2, 1 mM Oleic acid, 5% Glycerol PBS buffer for one hour prior to reading in BetaLux (Perkin Elmer).

### Plasmids and Viral Constructs

Human Glucagon Receptor-GFP construct was purchased from Origene. cAMP-response element luciferase construct was cloned by amplification of four tandem repeats of cAMP response element cloned at the proximal site of minimal basal reporter of nano-Luc. cAMP-LUC adenovirus for primary hepatocytes was purchased from Vector Biolabs. GCGR extracellular domain was cloned into pFastBac shuttle vector. During cloning hexahistadine and WELQ protease site was added for protein purification. Murine aP2 gene was cloned into pet21+ vector after the hexahistadine tag and TEV protease site for ease of purification.

### RNA Extraction and Quantitative PCR

RNA was extracted using Trizol reagent (Invitrogen) using manufacturer's instructions and quantitative PCR performed using the previously published primer sequences (Cao et al., Cell Metab. 2013 May 7; 17(5):768-778).

Animals and Cells

Animals were cared for in the USDA-inspected Harvard Animal Facility under federal, state, local and NIH guidelines for animal care. Male C57BL/6 mice (10-12 weeks) were obtained from the Jackson Laboratory. HepG2-C3A cells were obtained from American Type Culture Collection (ATCC). Cells were cultured in complete medium (DMEM, 4.5 g/L glucose, 10% fetal bovine serum (Atlanta Bio)), and MEM sodium pyruvate (1 mM). Primary hepatocytes were isolated from C57BL/6J mice and cultured in 100 U/mL penicillin G sodium and 100 μg/mL streptomycin (Pen/Strep). CHO/K1 cells were cultured in DMEM:F12 and 5% cosmic calf serum (Thermo Scientific). All media supplements were from Invitrogen.

### Cell Transfections, Stable Cell Line Generation and Viral Infection

Plasmids were transfected using Lipofectamine (Invitrogen) according to manufacturer's instructions. Transiently transfected cells were grown in appropriate selection antibiotics where applicable for stable cell line generation. Following three passages under selection media, single cell colonies were picked and expanded for experimentation after validation.

Statistical Analysis

All plots show mean values, and error bars represent the SEM for line graphs and SD for bar graphs. Comparisons of mean values of two groups were performed using unpaired Student's t tests unless indicated otherwise. One-way analysis of variance (ANOVA), followed by a Bonferroni post-test was used to compare >2 groups. Standard repeated measured test was performed where multiple measurements were taken from single animal. *, p<0.05; **, p<0.01; ***, p<0.001, ns. ‘not-significant’ unless otherwise indicated. Statistical analysis was performed using GraphPad Prism software v6.0 (San Diego, Calif.).

### aP2 Synergistically Activates Gluconeogenic Programming of Glucagon Actions

It has been well established that hypoglycemia counterregulatory hormones, mainly glucagon, epinephrine, cortisol, and growth hormone, act synergistically and share a common beta-adrenergic stimulus for secretion (Bolli et al., Diabetes. 1982 July; 31(7):641-647). It has been also noted that lipolytic signals following beta adrenergic activation contribute to this synergistic activity (Souza et al., Braz J Med Biol Res. 1994 December; 27(12):2883-2887), but lipid infusions fail to do so (Haywood et al., Am J Physiol Endocrinol Metab. 2009 July; 297(1): E50-56; and Antoniades et al., The Lancet. 1967 March; 289(7490):602-604), suggesting that an adipose tissue derived factor might mitigate these effects. Since circulating aP2 levels are regulated by beta adrenergic signaling (Cao et al., Cell Metab. 2013 May 7; 17(5):768-778) and contribute to hepatic glucose production (Cao et al., Cell Metab. 2013 May 7; 17(5):768-778), the hypothesis that aP2 synergistically works with glucagon to activate hepatic glucose production was tested.

To directly test this hypothesis, the effects of the main insulin counterregulatory hormone, glucagon, and the combined effect of aP2 and glucagon in isolated primary hepatocytes was first examined (FIGS. 1A and 1B). In this setup, addition of aP2 to glucagon further increased the expression of gluconeogenic genes beyond what glucagon alone did. Without wishing to be bound to any one theory, this supports a synergistic role for aP2 in gluconeogenic programming. This gluconeogenic gene expression is also consistent with functional assays in which hepatic glucose production in primary hepatocytes (FIG. 1C) and glycogenolysis in hepatoma cell line (FIG. 1D) are increased by aP2 synergizing with glucagon. To further examine the downstream signaling events involved in the process, and to study the effect of aP2 on glucagon actions, a human glucagon receptor expressing cAMP reporter system was constructed in CHO-K1 cells. As seen in FIG. 1E, aP2 addition increases the potency of glucagon more than one order of magnitude (Log₁₀ EC₅₀ glucagon −8.215±0.1556; glucagon+aP2−9.698±0.1448 M±S.E.M.). This observation is also consistent with results in primary hepatocytes when cAMP activity is assayed with using adenovirus mediated cAMP reporter luciferase (FIG. 1F).

### aP2 is an Allosteric Enhancer of the Glucagon Receptor.

Given the ability of aP2 to potentiate glucagon signal transduction and metabolic actions, further studies were conducted to determine whether aP2 has an upstream role in activation of the glucagon receptor. A G6Pc promoter driven reporter assay was utilized. As presented in FIG. 2A, the synergistic actions of aP2 and glucagon on G6Pc promoter activity are only present when the reporter plasmid is co-transfected with the glucagon receptor. Whether aP2 can act as an allosteric enhancer for glucagon on its receptor was tested using a baculovirus expression system. The extracellular domain of GCGR was expressed (GCGR-ecd) (Wu et al., Protein Expr Purif. 2013 June; 89(2):232-240) and the effect of aP2 on glucagon binding kinetics to GCGR-ecd was examined using the BLITZ biolayer interferometry system. The addition of aP2 caused a significant increase in the association and decrease in the dissociation rate of glucagon to GCGR-ecd (FIG. 2B), resulting in an order of magnitude decrease in the dissociation constant (Kdₐₚₚ glucagon 1.76e-007M; Kdₐₚₚ glucagon+aP2 5.41e-008M). These results are consistent with the effect that was observed for cAMP activity in vitro. Without wishing to be bound to any one theory, this provides direct evidence for increased activity of glucagon actions in the presence of aP2 (FIG. 1E). To further understand the role of aP2 as an allosteric modulator of glucagon actions, an allosteric inhibitor of glucagon receptor L-168,049 (Cascieri et al., J Biol Chem. 1999 Mar. 26; 274(13):8694-8697) was assessed for its ability to inhibit the actions of aP2. Addition of L-168,049 mitigated the synergetic effects of aP2 on glucagon's actions on the glucagon receptor (FIGS. 2C and 2D). Addition of L-168,049 also caused the loss of the ability to respond to aP2 and glucagon. Without wishing to be bound to any one theory, this suggests that aP2 may be binding to the allosteric site of the glucagon receptor (FIGS. 2E and 2F).

aP2 Directly Interacts with Glucagon

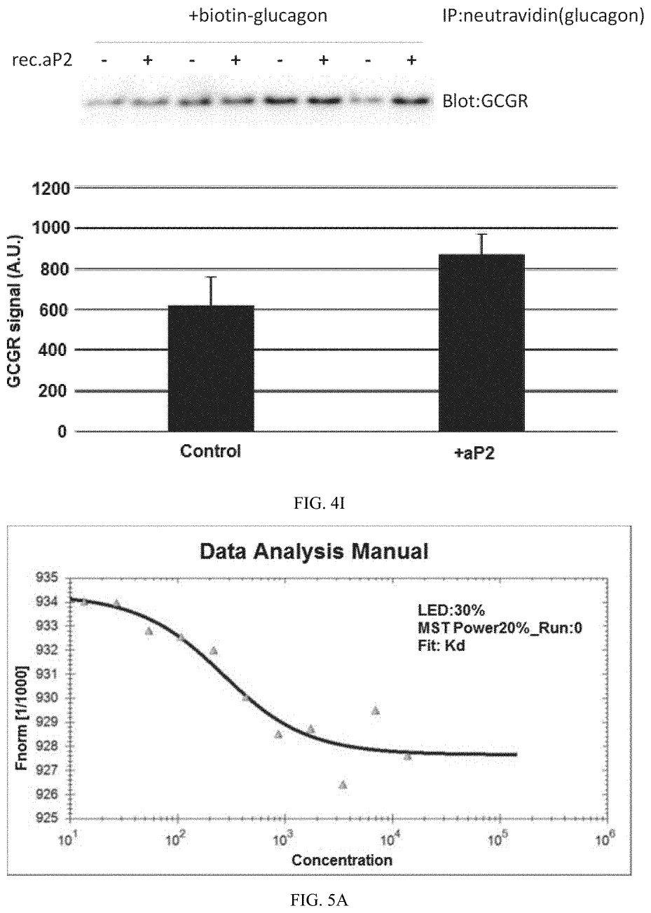

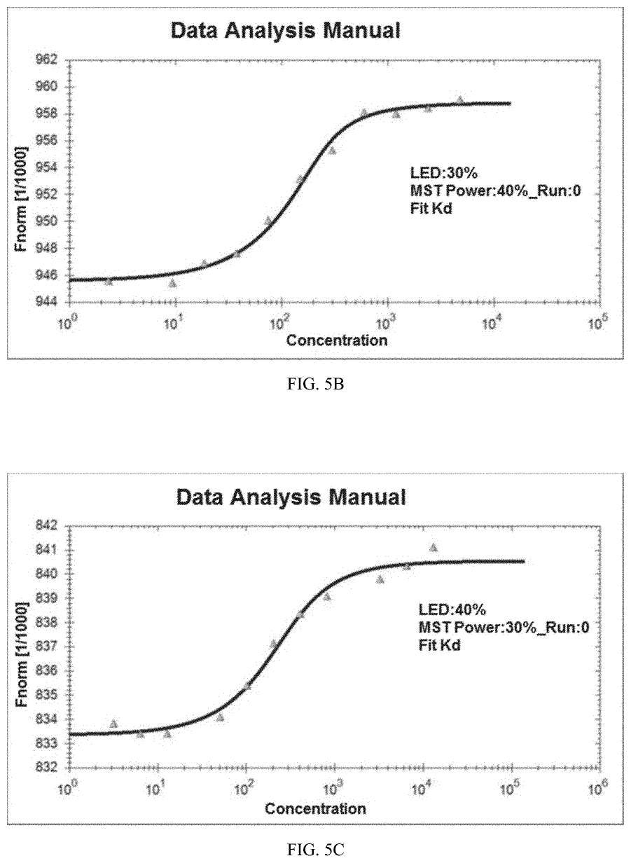

To understand the mechanisms by which aP2 increases glucagon actions, the possibility of a physical interaction of aP2 and glucagon was explored. A series of binding assays were utilized. First, using biolayer interferometry, a direct interaction of aP2 with biotinylated glucagon was demonstrated (FIG. 3A). Next, a scintillation proximity assay was used, in which ¹²⁵I-glucagon interacted with biotinylated aP2 (FIG. 3B). Using these complimentary tagged proteins, similar affinities were achieved (Kdₐₚₚ 2.34 μM and 2.62 μM respectively). To further investigate this protein-peptide interaction in a tag-free system, isothermal titration calorimetry was employed as a gold standard binding assay which measures heat liberated from binding events in solution. This approach revealed direct glucagon/aP2 binding (FIG. 3C). These measurements are also consistent with previous binding studies described. To address the physiological relevance of this interaction, it was first attempted to pull down the endogenous complex from circulation. Following incubation with anti-aP2 antibody coated magnetic beads, a glucagon signal was detected in the serum of wild type mice using HRP conjugated anti-glucagon antibody (FIGS. 3D, 3E, and 3F). In the absence of aP2 (aP2^{-/-} serum or antibody depleted wild-type sera), there was a minimal amount of glucagon signal (indistinguishable from non-specific binding signal). Addition of recombinant aP2 to aP2^{-/-} sera resulted in appreciably higher glucagon signal recovered by anti-aP2 antibodies, but did not reach statistical significance. These results were independent of respective levels of glucagon in the wild-type and aP2^{-/-} sera (189±20, 210±41 pg/mL respectively). Additionally, biotinylated glucagon was used as bait to pull down endogenous aP2 from wild-type and aP2^{-/-} sera (FIG. 3M). Taken together and without wishing to be bound to any one theory, these results indicate that the ability of aP2 to bind glucagon has a physiological relevance and that the glucagon/aP2 protein complex occurs naturally. Microscale Thermophoresis (MST), which allows for interaction analysis of biomolecules using thermophoresis, confirmed the lack of additional in vivo adapter proteins. Changes in the properties of molecules (e.g., size, charge, and solvation entropy of molecules) due to the binding between molecules change molecules' thermophoresis. MST can measure the binding affinity between molecules based on molecules' thermophoretic motion by measuring interactions directly in solution without immobilizing molecules to a surface. Using MST, it was shown that aP2 binds to glucagon (Kd of about 214 nM), glucagon binds to the glucagon receptor (Kd of about 36.7 nM), and aP2 binds to the glucagon receptor (Kd of about 15.4 to about 120 nM) (FIGS. 5A-ED).

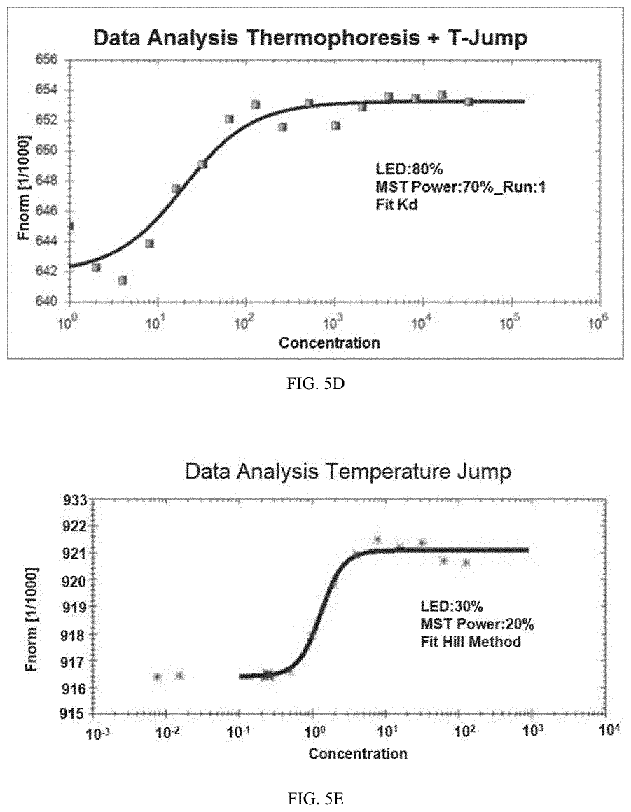

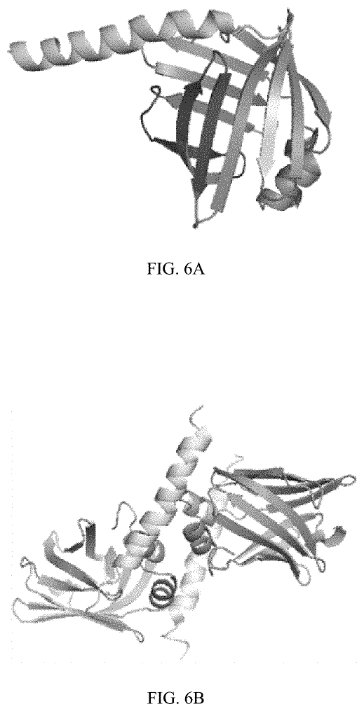

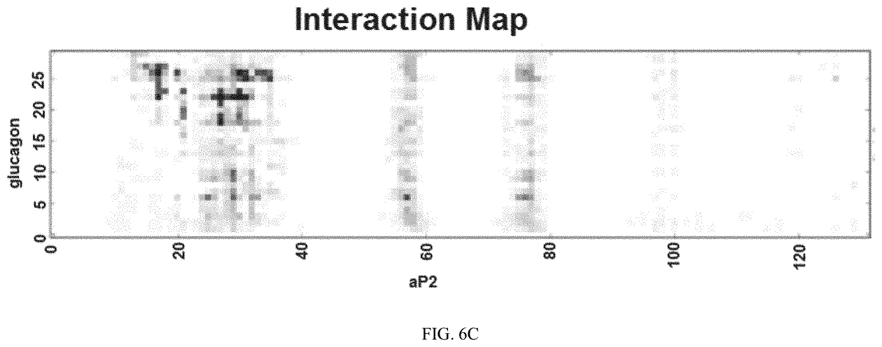

To give a starting point of possible residues of interaction, bioinformatics tools have been employed to assist in designing point mutations. For a completely unbiased prediction, several prediction algorithms have been employed (Pierce et al., Bioinformatics. 2014 Mar. 12; Cheng et al, Proteins. 2007 Aug. 1; 68(2):503-515; Comeau et al., Bioinformatics. 2004 Jan. 1; 20(1):45-50; and Jimenez-Garcia et al., Bioinformatics. 2013 Jul. 1; 29(13):1698-1699) to give all of the possible prediction patterns. Moreover, as multiple crystallographic conformations for aP2 have been reported (LaLonde et al., Biochemistry. 1994 Apr. 26; 33(16):4885-4895), the possibility of multiple stoichiometric ratios of aP2 and glucagon have also been included to the searches. As different algorithms for protein-protein dockings have different efficiencies of predicting different tertiary structures (Janin et al., Proteins. 2003 Jul. 1; 52(1):2-9), known structures of aP2 and glucagon with known binding partners have been validated. It was found that all three servers used for the predictions predicted binding of aP2 to glucagon with RMSD of 99.7% confidence or better, suggesting that the predictions by servers would produce an outcome as close to the observed interaction as possible. Of the 14,000 predictions generated between the three servers and two possible interaction ratios, the CONS-COCOMAPS (Vangone et al., Bioinformatics. 2011 Aug. 27; btr484) server has been used to map distribution frequency of the possible interacting sites (FIG. 6C). The first alpha helix and Phe57 site have been identified as potential sites of interaction. To further elucidate the potential sites of interaction, a triple mutant aP2 with three point mutations was made (N59, E61, and K79). Binding curves were generated using human aP2, mutant aP2 with glucagon and an anti-aP2 antibody. It was shown that this mutant protein has lower binding affinity as measured with an Octet system (FIGS. 3G-3J). In addition, truncations were made to the glucagon protein and binding was tested to aP2 in both wild type and aP2^{-/-} mice. From these experiments it was shown that residues 22-29 of glucagon are important for binding aP2 (FIG. 3L).

To elucidate the role of aP2 in glucagon binding to GCGR, enriched plasma membrane fractions from wild-type and GCGR deficient (GCGR^{fl/fl-Alb Cre}) mouse livers were isolated by differential centrifugation. The plasma membrane was incubated with a fixed biotinylated glucagon concentration (20 nM) and increasing amounts of aP2. Plasma membrane and +/−aP2 and glucagon were then incubated in wheat germ agglutinin coated plates and washed extensively to remove unbound proteins. HRP conjugated streptavidin was used to detect glucagon (FIG. 4A). To show that glucagon binding to the GCGR receptor requires aP2 in vivo, ¹²⁵I labeled glucagon was administered via the portal vein of wild-type, aP2^{-/-} (with and without recombinant aP2) and GCGR^{fl/fl-Alb Cre} mice. The animals were euthanized and perfused with cold PBS for 5 minutes transcardially 5 minutes following administration. The organs were harvested at the end of the perfusion, digested, and radiation was counted with liquid scintillation counter (FIGS. 4B, 4C, 4D, and 4E). As shown in FIG. 4F, aP2 increases GCGR.ecd binding to glucagon. Pull-down experiments were also completed. aP2 was pulled down using GCGR as bait. Livers from wild-type mice were homogenated in lysis buffer and incubated overnight with recombinant aP2 (10 ug), glucagon (1 ug), and GCGR antibody coupled to magnetic beads. After centrifugation, aP2, glucagon, and glucagon+aP2 signal was measured in the pellet and supernatant (FIGS. 4G and 4H). Next GCGR was pulled down using biotinylated glucagon as bait. Livers from wild-type mice were homogenated in lysis buffer and incubated overnight with recombinant aP2 (10 ug), biotin-glucagon (1 ug), and Neutravidin coupled magnetic beads. The GCGR signal was measured and was shown to be significantly higher with aP2 (FIG. 4I).

### aP2 is Required for Glucagon Actions In Vivo

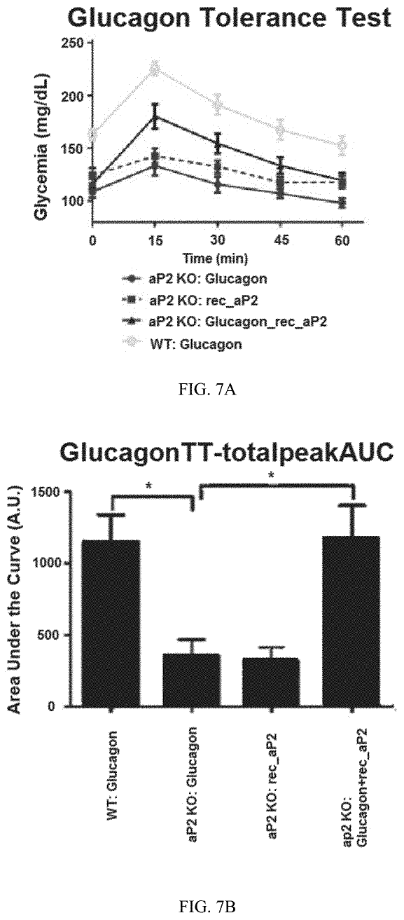

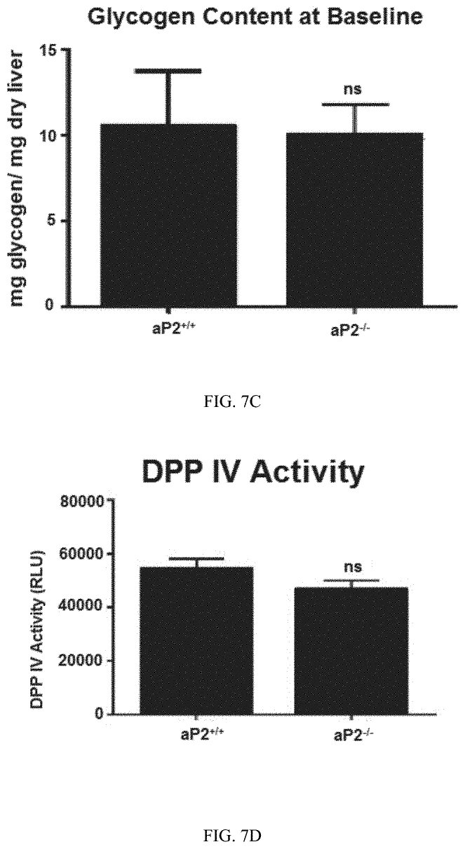

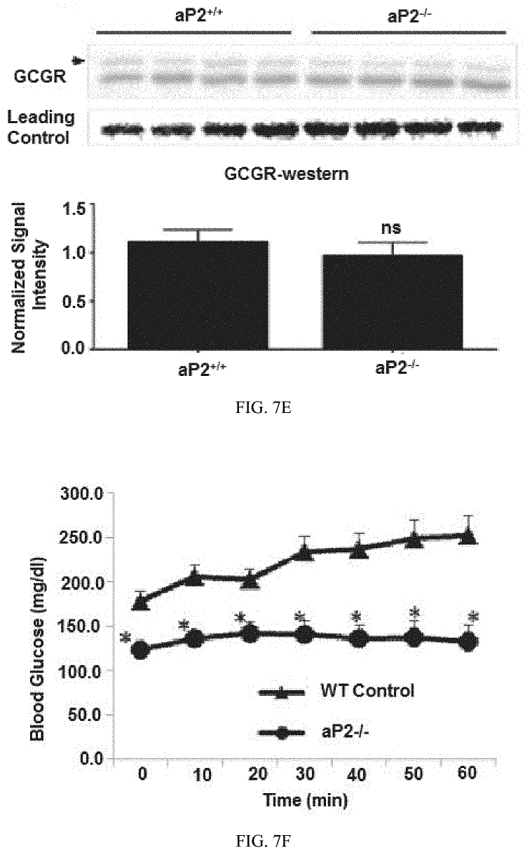

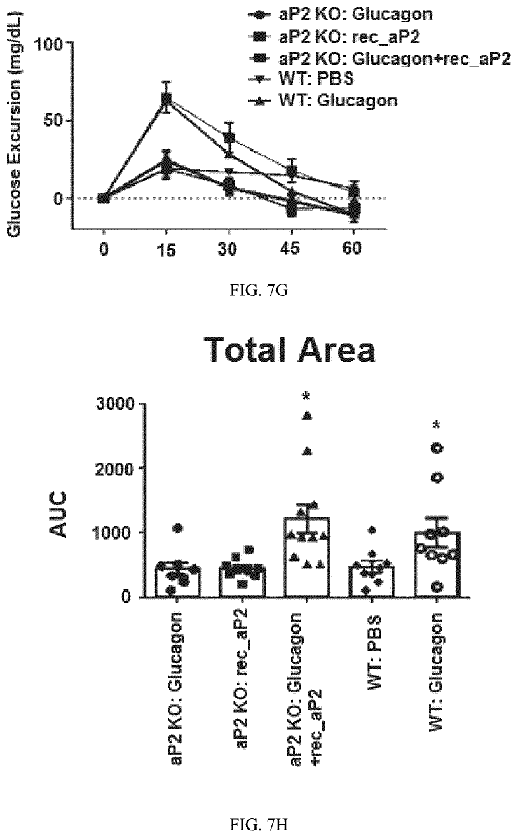

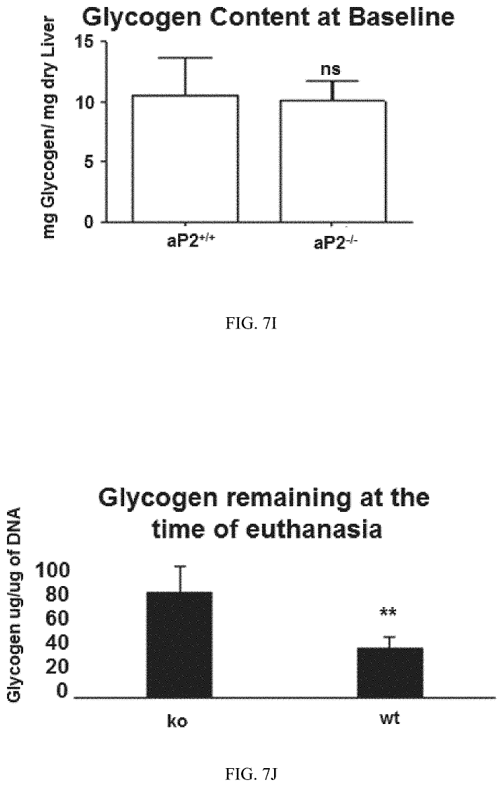

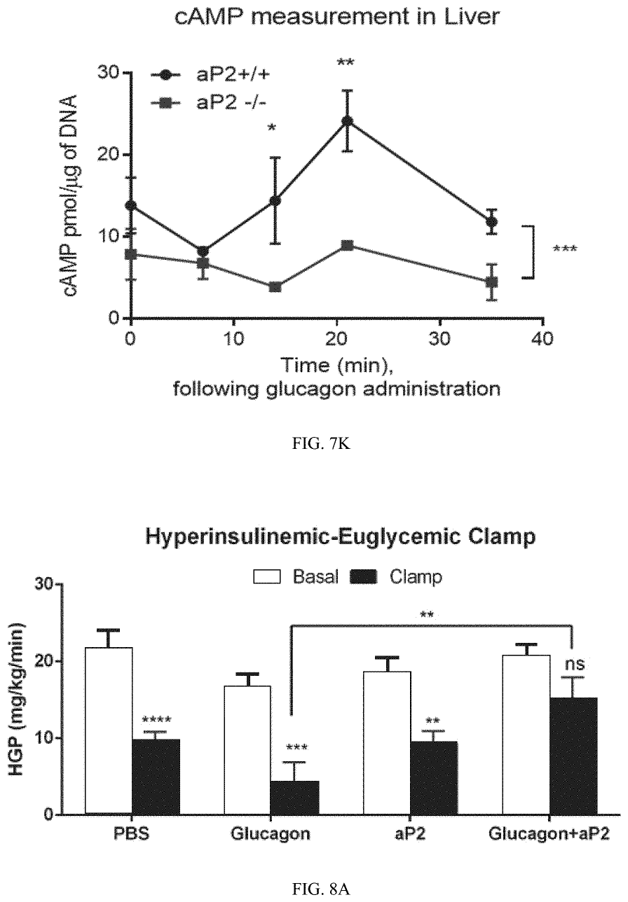

Glucagon was injected into aP2 deficient and wild-type mice, and their glycaemia was followed as a measure of glucagon action (Gelling et al., Proc. Natl. Acad. Sci. USA. 2003 Feb. 4; 100(3):1438-1443). Surprisingly, aP2 deficient animals had little to no-response to glucagon and aP2 administration alone, whereas simultaneous glucagon and recombinant aP2 injection to aP2 knockout mice was able to restore glucagon responsiveness (FIGS. 7A, 7B). FIGS. 7G and 7H show the glucose tolerance test of aP2 knockout and wild-type mice treated with either PBS, glucagon, or glucagon and aP2. Without wishing to bound by any one theory, this shows aP2 knockout mice only respond to glucagon treatment with the addition of aP2. No difference in the liver glycogen content at baseline or the expression of glucagon receptor (FIGS. 7C, 7E, and 7I) was observed in livers of aP2′ mice excluding inherent defects in glucagon signal transduction or glycogen content. FIG. 7J shows the glycogen remaining at the time of euthanasia. When compared with the baseline measurements, the glucose excursion seen in FIG. 7G is due to glycogen breakdown. Moreover, following glucagon administration, circulating glucagon levels reached supraphysiological levels in both WT and aP2-KO mice excluding rapid degradation or decreased bioavailability of glucagon as an explanation for the lack of glucagon action in aP2-KO mice (unpublished results). In addition, the activity of DPP4, the primary glucagon peptidase (Hinke et al., J Biol Chem. 2000 Feb. 11; 275(6):3827-3834), was not increased in sera of aP2-KO mice, further ruling out a rapid degradation scenario (FIG. 7D). Furthermore, pharmacological doses of glucagon under pancreatic clamp conditions were infused with basal insulin levels through a jugular vein catheter (FIG. 7F). Under these conditions, wild type animals had a constant increase in their blood glucose levels compared to their non-responsive aP2 deficient littermates despite having increased glycemia at baseline. Taken together and without wishing to be bound to any one theory, these results rule out hepatic defects as a plausible mechanism for glucagon hypo-responsiveness observed in aP2 deficient mice.

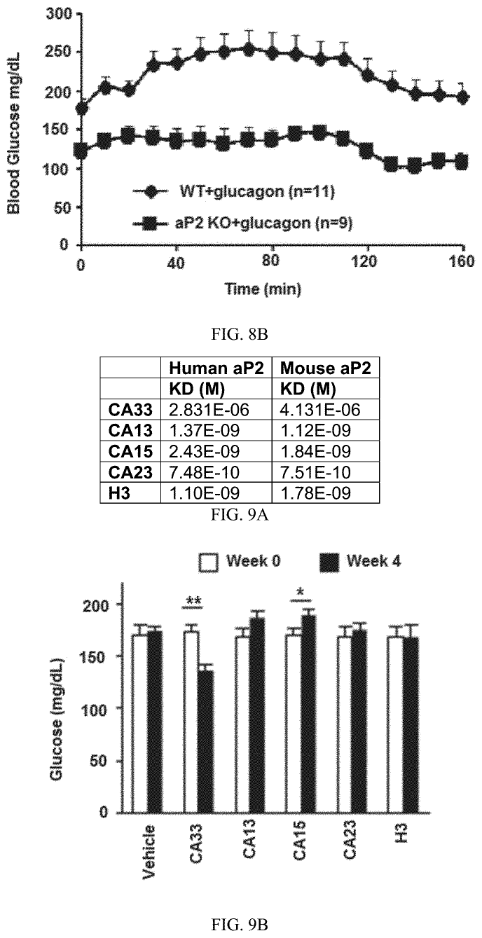

Lastly, hyper-insulinemic-pancreatic clamp studies were performed to measure the counter-regulatory activity of glucagon, aP2, and glucagon and aP2 in the aP2 deficient background (FIG. 8A). In this setup, a significant enhancement of hepatic glucose production in glucagon or aP2 alone compared to vehicle administration was not seen, whereas simultaneous aP2 and glucagon administration fully restored counterregulatory response to insulin. Additionally, it was shown that even under constant infusion of glucagon, there is no glucose production in response to glucagon in aP2-deficient mice, which shows that aP2 is required for glucagon action in vivo (FIG. 8B).

### Example 2: Preparation of an Illustrative Monoclonal Antibody Targeting Secreted aP2/Glucagon/aP2 Protein Complex

Animals

Animal care and experimental procedures were performed with approval from animal care committees of Harvard University. Male mice (leptin-deficient (ob/ob) and diet induced obese (DIO) mice with C57BL/6J background) were purchased from The Jackson Laboratory (Bar Harbor, Me.) and kept on a 12-hour light/dark cycle. DIO mice with C57BL/6J background were maintained on high-fat diet (60% kcal fat, Research Diets, Inc., D12492i) for 12 to 15 weeks before starting treatment except in clamp studies, for which they were on HFD for 20 weeks. Leptin-deficient (ob/ob) mice were maintained on regular chow diet (RD, PicoLab 5058 Lab Diet). Animals used were 18 to 31 weeks of age for dietary models and 9 to 12 weeks of age for the ob/ob model. In all experiments, at least 7 mice in each group were used, unless otherwise stated in the text. The mice were treated with 150 μl PBS (vehicle) or 1.5 mg/mouse (˜33 mg/kg) anti-aP2 monoclonal antibody in 150 μl PBS by twice weekly subcutaneous injections for 3 to 5 weeks. Before and after the treatment, blood samples were collected from the tail after 6 hours of daytime food withdrawal. Body weights were measured weekly in the fed state. Blood glucose levels were measured weekly after 6 hours of food withdrawal or after 16 hours overnight fast. After 2 weeks of treatment, glucose tolerance tests were performed by intraperitoneal glucose injections (0.75 g/kg for DIO, 0.5 g/kg for ob/ob mice). After 3 weeks of treatment, insulin tolerance tests were performed in DIO mice by intraperitoneal insulin injections (0.75 IU/kg). After 5 weeks of treatment, hyperinsulinemic-euglycemic clamp experiments were performed in DIO mice as previously described (Furuhashi et al., (2007) Nature 447, 959-965; Maeda et al., (2005) Cell metabolism 1, 107-119).

Metabolic cage (Oxymax, Columbus Instruments) and total body fat measurement by dual energy X-ray absorptiometry (DEXA; PIXImus) were performed as previously described (Furuhashi et al., (2007) Nature 447, 959-965).

### Production and Administration of Anti-aP2/Glucagon/aP2 Protein Complex Antibodies

CA13, CA15, CA23 and CA33 (Rabbit Ab 909) were produced and purified by UCB. New Zealand White rabbits were immunized with a mixture containing recombinant human and mouse aP2 (generated in-house in E. coli: accession numbers CAG33184.1 and CAJ18597.1, respectively). Splenocytes, peripheral blood mononuclear cells (PBMCs) and bone marrow were harvested from immunized rabbits and subsequently stored at −80° C. B cell cultures from immunized animals were prepared using a method similar to that described by Zubler et al., (“Mutant EL-4 thymoma cells polyclonally activate murine and human B cells via direct cell interaction”, J Immunol 134, 3662-3668 (1985)). After a 7-day incubation, antigen-specific antibody-containing wells were identified using a homogeneous fluorescence-linked immunosorbent assay with biotinylated mouse or human aP2 immobilized on Superavidin™ beads (Bangs Laboratories) and a goat anti-rabbit IgG Fcγ-specific Cy-5 conjugate (Jackson ImmunoResearch). To identify, isolate, and recover the antigen-specific B-cell from the wells of interest, fluorescent foci method was used (Clargo et al., (2014) mAbs 6, 143-159). This method involved harvesting B cells from a positive well and mixing with paramagnetic streptavidin beads (New England Biolabs) coated with biotinylated mouse and human aP2 and goat anti-rabbit Fc fragment-specific FITC conjugate (Jackson ImmunoResearch). After static incubation at 37° C. for 1 h, antigen-specific B cells could be identified due to the presence of a fluorescent halo surrounding that B cell. Individual antigen-specific antibody secreting B cells were viewed using an Olympus IX70 microscope, were picked with an Eppendorf micromanipulator, and deposited into a PCR tube. Variable region genes from these single B-cells were recovered by RT-PCR, using primers that were specific to heavy- and light-chain variable regions. Two rounds of PCR were performed, with the nested 2° PCR incorporating restriction sites at the 3′ and 5′ ends, allowing cloning of the variable region into a variety of expression vectors: mouse γ1 IgG, mouse Fab, rabbit γ1 IgG (VH) or mouse kappa and rabbit kappa (VL). Heavy- and light-chain constructs were transfected into HEK-293 cells using Fectin 293 (Invitrogen) and recombinant antibody expressed in 6-well plates. After 5 days' expression, supernatants were harvested and the antibody was subjected to further screening by biomolecular interaction analysis using the BiaCore system to determine affinity and epitope bin.

Mouse anti-aP2 monoclonal antibody H3 was produced by the Dana Farber Cancer Institute Antibody Core Facility. Female C57BL/6 aP2−/− mice, 4-6 weeks old, were immunized by injection of full-length human aP2/FABP4-Gst recombinant protein was suspended in Dulbecco's phosphate buffered saline (PBS; GIBCO, Grand Island, N.Y.) and emulsified with an equal volume of complete Freund's adjuvant (Sigma Chemical Co., St. Louis, Mo.). Spleens were harvested from immunized mice and cell suspensions were prepared and washed with PBS. The spleen cells were counted and mixed with SP 2/0 myeloma cells (ATCC No. CRL8-006, Rockville, Md.) that are incapable of secreting either heavy or light chain immunoglobulins (Kearney et al., (1979) Journal of Immunology 123, 1548-1550) at a spleen:myeloma ratio of 2:1. Cells were fused with polyethylene glycol 1450 (ATCC) in 12 96-well tissue culture plates in HAT selection medium according to standard procedures (Kohler et al., (1975) Nature 256, 495-497). Between 10 and 21 days after fusion, hybridoma colonies became visible and culture supernatants were harvested then screened by western blot on strep-His-human-aP2/FABP4. A secondary screen of 17 selected positive wells was done using high-protein binding 96-well EIA plates (Costar/Corning, Inc. Corning, N.Y.) coated with 50 μl/well of a 2 μg/ml solution (0.1 μg/well) of strep-His-human-aP2/FABP4 or an irrelevant Gst-protein and incubated overnight at 4° C.). Positive hybridomas were subcloned by limiting dilution and screened by ELISA. Supernatant fusions were isotyped with Isostrip kit (RocheDiagnostic Corp., Indianapolis, Ind.).

Large-scale transient transfections were carried out using UCB's proprietary CHOSXE cell line and electroporation expression platform. Cells were and maintained in logarithmic growth phase in CDCHO media (LifeTech) supplemented with 2 mM Glutamax at 140 rpm in a shaker incubator (Kuhner A G, Birsfelden, Switzerland) supplemented with 8% CO2 at 37° C. Prior to transfection, the cell numbers and viability were determined using CEDEX cell counter (Innovatis AG. Bielefeld, Germany) and 2×108 cells/ml were centrifuged at 1400 rpm for 10 minutes. The pelleted cells were washed in Hyclone MaxCyte buffer (Thermo Scientific) and respun for a further 10 minutes and the pellets were re-suspended at 2×108 cells/ml in fresh buffer. Plasmid DNA, purified using QIAGEN Plasmid Plus Giga Kit® was then added at 400 μg/ml. Following electroporation using a Maxcyte STX® flow electroporation instrument, the cells were transferred in ProCHO medium (Lonza) containing 2 mM Glutamax and antibiotic antimitotic solution and cultured in wave bag (Cell Bag, GE Healthcare) placed on Bioreactor platform set at 37° C. and 5% CO2 with wave motion induced by 25 rpm rocking.

Twenty-four hours post transfection a bolus feed was added and the temperature was reduced to 32° C. and maintained for the duration of the culture period (12-14 days). At day 4, 3 mM sodium butryrate (n-BUTRIC ACID sodium salt, Sigma B-5887) was added to the culture. At day 14, the cultures were centrifuged for 30 minutes at 4000 rpm and the retained supernatants were filtered through 0.22 μm SARTO BRAN-P (Millipore) followed by 0.22 μm Gamma gold filters. CHOSXE harvest expressing mouse monoclonal antibody (mAb) was conditioned by addition of NaCl (to 4M). The sample was loaded onto a protein A MabSelect Sure packed column (GE-healthcare) equilibrated with 0.1M Glycine+4M NaCl pH8.5 at 15 ml/min. The sample was washed with 0.1M Glycine+4M NaCl pH8.5 and an additional wash step was performed with 0.15M Na2HPO4 pH 9. The UV absorbance peak at A280 nm was collected during elution from the column using 0.1M sodium citrate pH 3.4 elution buffer and then neutralized to pH 7.4 by addition of 2M Tris-HCl pH 8.5. The mAb pool from protein A was then concentrated to suitable volume using a minisette Tangential Flow Filtration device before being purified further on a HiLoad XK 50/60 Superdex 200 prep grade gel filtration column (GE-healthcare). Fractions collected were then analyzed by analytical gel filtration technique for monomer peak and clean monomer fractions pooled as final product. The final product sample was then characterized by reducing and non-reduced SD S-PAGE and analytical gel filtration for final purity check. The sample was also tested and found to be negative for endotoxin using a LAL assay method for endotoxin measurements. The final buffer for all mAbs tested was PBS. For in vivo analysis, purified antibodies were diluted in saline to 10 mg/ml and injected at a dose of 1.5 mg/mouse (33 mg/kg) into ob/ob and WT mice on high-fat diet.

### Measurement of Antibody Affinity

The affinity of anti-aP2 binding to aP2 (recombinantly generated in E. coli as described below) was determined by biomolecular interaction analysis, using a Biacore T200 system (GE Healthcare). Affinipure F(ab′)2 fragment goat anti-mouse IgG, Fc fragment specific (Jackson ImmunoResearch Lab, Inc.) in 10 mM NaAc, pH 5 buffer was immobilized on a CMS Sensor Chip via amine coupling chemistry to a capture level between 4500-6000 response units (RU) using HBS-EP+(GE Healthcare) as the running buffer. Anti-aP2 IgG was diluted to between 1-10 μg/ml in running buffer. A 60 s injection of anti-aP2 IgG at 10 μl/min was used for capture by the immobilized anti-mouse IgG, Fc then aP2 was titrated from 25 nM to 3.13 nM over the captured anti-aP2 for 180 s at 30 μl/min followed by 300 s dissociation. The surface was regenerated by 2×60 s 40 mM HCl and 1×30 s 5 mM NaOH at 10 μl/min. The data were analyzed using Biacore T200 evaluation software (version 1.0) using the 1:1 binding model with local Rmax. For CA33, 60 s injection of the antibody at 10 μl/min was used for capture by the immobilized anti-mouse IgG, Fc then aP2 was titrated from 40 μM to 0.625 μM over the captured anti-aP2 for 180 s at 30 μl/min followed by 300 s dissociation. The surface was regenerated by 1×60 s 40 mM HCl, 1×30 s 5 mM NaOH and 1×60 s 40 mM HCl at 10 μl/min. Steady state fitting was used to determine affinity values.

### Antibody Cross-Blocking

The assay was performed by injecting mouse aP2 in the presence or absence of mouse anti-aP2 IgG over captured rabbit anti-aP2 IgG. Biomolecular interaction analysis was performed using a Biacore T200 (GE Healthcare Bio-Sciences AB). Anti-aP2 rabbit IgG transient supernatants were captured on the immobilized anti-rabbit Fc surfaces (one supernatant per flowcell) using a flow rate of 10 μl/min and a 60 s injection to give response levels above 200 RU. Then mouse aP2 at 100 nM, 0 nM or mouse aP2 at 100 nM plus mouse anti-aP2 IgG at 500 nM were passed over for 120 s followed by 120 s dissociation. The surfaces were regenerated with 2×60 s 40 mM HCl and 1×30 s 5 mM NaOH.

### FABP Cross-Reactivity

The recombinant human proteins aP2 (generated at UCB in E. coli (see method below)), hFABP3 (Sino Biological Inc.) and hFABP5/hMal1 (Sino Biological Inc.) were biotinylated in a 5-fold molar excess of EZ-Link® Sulfo-NHS-LC-Biotin (Thermo Fisher Scientific) and purified from unbound biotin using a Zeba desalting column (Thermo Fisher Scientific). All binding studies were performed at 25° C. using a ForteBio Octet RED384 system (Pall ForteBio Corp.). After a 120 s baseline step in PBS containing 0.05% Tween 20, pH7.4 (PBS-T), Dip and Read™ streptavidin (SA) biosensors (Pall ForteBio Corp.) were loaded with biotinylated recombinant haP2, hFABP3 or hFABP5/hMal1 at 60 nM for 90 s. After a 60 s stabilization step in PBS-T, each FABP-loaded biosensor was transferred to a sample of monoclonal antibody at 800 nM and association was measured for 5 min. Biosensors were then transferred back to PBS-T for 5 min. to measure dissociation. Non-specific binding of antibodies was monitored using unloaded biosensor tips. Maximal association binding i.e., once signal had plateaued, minus background binding, was plotted for each antibody/FABP combination.

### aP2 Expression and Purification

Mouse (or human) aP2 cDNA optimized for expression in E. coli was purchased from DNA 2.0 (Menlo Park, Calif.) and subcloned directly into a modified pET28a vector (Novagen) containing an in-frame N-terminal 10 His-tag followed by a Tobacco Etch Virus (TEV) protease site. Protein was expressed in the E. coli strain BL21DE3 and purified as follows. Typically, cells were lysed with a cooled cell disruptor (Constant Systems Ltd.) in 50 ml lysis buffer (PBS (pH 7.4) containing 20 mM imidazole) per liter of E. coli culture supplemented with a Complete protease inhibitor cocktail tablet, EDTA-free (Roche, Burgess Hill). Lysate was then clarified by high-speed centrifugation (60000 g, 30 minutes, 4° C.). 4 ml/Ni-NTA beads (Qiagen) were added per 100 ml cleared lysate and tumbled for 1 h at 4° C. Beads were packed in a Tri-Corn column (GE Healthcare) attached to an AKTA FPLC (GE Life Sciences) and protein eluted in a buffer containing 250 mM imidazole. Fractions containing protein of interest as judged by 4-12% Bis/Tris NuPage (Life Technologies Ltd.) gel electrophoresis were dialyzed to remove imidazole and treated with TEV protease at a ratio of 1 mg per 100 mg protein. After overnight incubation at 4° C. the sample was re-passed over the Ni/NTA beads in the Tri-Corn column. Untagged (i.e. TEV cleaved) aP2 protein did not bind to the beads and was collected in the column flow through. The protein was concentrated, and loaded onto an S75 26/60 gel filtration column (GE healthcare) pre-equilibrated in PBS, 1 mM DTT. Peak fractions were pooled and concentrated to 5 mg/ml. Six ml of this protein was then extracted and precipitated with acetonitrile at a ratio of 2:1 to remove any lipid. Following centrifugation at 16,000 g for 15 mins the acetonitrile+ buffer was removed for analysis of original lipid content. The pellet of denatured protein was then resuspended in 6 ml of 6M GuHCl PBS+2 μMoles palmitic acid (5:1 ratio of palmitic acid to aP2) and then dialyzed two times against 5L PBS for 20 hrs at 4° C. to allow refolding. Following centrifugation to remove precipitate (16000 g, 15 minutes) protein was gel filtered using a S75 26/20 column in PBS to remove aggregate. Peak fractions were pooled and concentrated to 13 mg/ml.

### aP2 Crystallography

Purified mouse aP2 was complexed with CA33 and H3 Fab (generated at UCB by conventional methods) as follows. Complex was made by mixing 0.5 ml of aP2 at 13 mg/ml with either 0.8 ml of CA33 Fab at 21.8 mg/ml or 1.26 ml of H3 Fab at 13.6 mg/ml (aP2:Fab molar ratio of 1.2:1). Proteins were incubated at RT for 30 minutes then run on an S75 16/60 gel filtration column (GE Healthcare) in 50 mM Tris pH7.2, 150 mM NaCl+5% glycerol. Peak fractions were pooled and concentrated to 10 mg/ml for crystallography.

Sitting-drop crystallization trials were set up using commercially available screening kits (QIAGEN). Diffraction-quality crystals were obtained directly in primary crystallization screening without any need to optimize crystallization conditions. For the aP2/CA33 complex the well solution contained 0.1M Hepes pH 7.5, 0.2M (NH₄)₂SO₄, 16% PEG 4K and 10% isopropanol. For the aP2/H3 complex the well solution contained 0.1M IVIES pH5.5, 0.15M (NH₄)₂SO₄ and 24% PEG 4K. Data were collected at the Diamond Synchrotron on i02 (λ=0.97949) giving a 2.9 Å dataset for aP2/CA33 and a 2.3 Å dataset for aP2/H3. Structures were determined by molecular replacement using Phaser (44) (CCP4) with aP2 and a Fab domain starting models. Two complexes were found to be in the asymmetric unit for aP2/CA33 and one for aP2/H3. Cycles of refinement and model building were performed using CNS (Brunger et al., (2007) Nature Protocols 2, 2728-2733) and coot (Emsley et al., (2004) Acta crystallographica. Section D, Biological crystallography 60, 2126-2132) (CCP4) until all the refinement statistics converged for both models. Epitope information described above was derived by considering atoms within 4A distance at the aP2/Fab contact surface. The data collection and refinement statistics are shown below. Values in parenthesis refer to the high-resolution shell.

| | | |
|---|---|---|
| Structure | aP2-CA33 | aP2-H3 |
| Space group | P 1 2₁ 1 | P 1 2₁ 1 |

Cell dimensions□□

| | | |
|---|---|---|
| a, b, c (Å) | 65.27, 101.95, 95.31 | 71.50, 66.04, 75.68 |
| □□□□□□□□□□□□ | 90.00, 90.03, 90.00 | 90.00, 111.67, 90.00 |
| (°) | | |

| | | | | |
|---|---|---|---|---|
| Resolution (Å) | 54.97-2.95 | (3.09-2.95) | 33.03-2.23 | (2.37-2.23) |
| R_{sym} or R_{merge} | 0.18 | (1.169) | 0.11 | (0.352) |
| I/□I | 8.3 | (2.9) | 6.8 | (1.7) |
| Completeness (%) | 99.2 | (98.9) | 98.6 | (98.4) |
| Redundancy | 6.2 | (6.3) | 2.6 | (2.6) |
| Refinement | | | | |

| | | |
|---|---|---|
| Resolution (Å) | 54.97-2.95 | 33.02-3.00 |
| No. reflections | 24898 | 13077 |
| R_{work}/R_{free} | 0.21/0.28 | 0.22/0.27 |

| No. atoms | | |
|---|---|---|
| Protein | 8632 | 4331 |
| Water | 0 | 0 |
| B-factors | | |

| | | |
|---|---|---|
| aP2 | (molecule 1) 58.3; | 27.5 |
| | (molecule 2) 64.6 | |
| Fab | (molecule 1) 52.9; | 22.5 |
| | (molecule 2) 52.5 | |

R.m.s. deviations

| | | |
|---|---|---|
| Bond lengths (Å) | 0.009 | 0.011 |
| Bond angles (°) | 1.42 | 1.67 |

Values in parenthesis refer to the high resolution shell.
R_{sym} = Σ|(I − <I>)|/Σ(I), where I is the observed integrated intensity, <I> is the average integrated intensity obtained from multiple measurements, and the summation is over all observed reflections. R_{work} = Σ| |F_{obs}| − k|F_{calc}| |/Σ|F_{obs}| , where F_{obs} and F_{calc} are the observed and calculated structure factors, respectively. R_{free} is calculated as R_{work} using 5% of the reflection data chosen randomly and omitted from the refinement calculations. Epitope information was derived by considering atoms within 4 Å distance at the aP2/Fab contact surface.

### Hyperinsulinemic-Euglycemic Clamp Studies and Hepatic Biochemical Assays

Hyperinsulinemic-euglycemic clamps were performed by a modification of a reported procedure (Cao et al., (2013) Cell Metab. 17, 768-778). Specifically, mice were clamped after 5 hours fasting and infused with 5 mU/kg/min insulin. Blood samples were collected at 10-min intervals for the immediate measurement of plasma glucose concentration, and 25% glucose was infused at variable rates to maintain plasma glucose at basal concentrations. Baseline whole-body glucose disposal was estimated with a continuous infusion of [3-³H]-glucose (0.05 μCi/min). This was followed by determination of insulin-stimulated whole-body glucose disposal whereby [3-³H]-glucose was infused at 0.1 μCi/min.

Total lipids in liver were extracted according to the Bligh-Dyer protocol (Bligh et al., (1959) Canadian J. Biochem. and Phys. 37, 911-917), and a colorimetric method used for triglyceride content measurement by a commercial kit according to manufacturer's instructions (Sigma Aldrich). Gluconeogenic enzyme Pck1 activity was measured by a modification of reported method (Petrescu et al., (1979) Analytical Biochem. 96, 279-281). Glucose-6-phosphatase (G6pc) activity was measured by a modification of Sigma protocol [EC 3.1.3.9]. Briefly, the livers were homogenized in lysis buffer containing 250 mM sucrose, Tris HCl and EDTA. Lysates were centrifuged at full speed for 15 min and the supernatant (predominantly cytoplasm) isolated. Then microsomal fractions were isolated by ultracentrifugation of cytoplasmic samples. Microsomal protein concentrations were measured by commercial BCA kit (Thermo Scientific Pierce). 200 mM glucose-6-phosphate (Sigma Aldrich) was pre-incubated in Bis-Tris. 150 μg microsomal protein or serial dilution of recombinant G6Pase were added and incubated in that solution for 20 min at 37° C. Then 20% TCA was added, mixed and incubated for 5 min at room temperature. Samples were centrifuged at full speed at 4° C. for 10 min, and the supernatant was transferred to a separate UV plate. Color reagent was added and absorbance at 660 nm was measured and normalized to standard curve prepared with serial dilution of recombinant glucose-6-phosphatase (G6pc) enzyme.

Plasma aP2, Mal1, FABP3, Adiponectin, Glucagon, and Insulin ELISAs

Blood was collected from mice by tail bleeding after 6 hours daytime or 16 hours overnight food withdrawal. Terminal blood samples were collected by cardiac puncture or collected from tail vein. The samples were spun in a microcentrifuge at 3,000 rpm for 15 minutes at 4° C. Plasma aP2 (Biovendor R&D), mall (Circulex Mouse Mal1 ELISA, CycLex Co., Ltd., Japan), FABP3 (Hycult Biotech, Plymouth Meeting, Pa.) glucagon, adiponectin (Quantikine ELISA, R&D Systems, Minneapolis, Minn.), and insulin (insulin-mouse ultrasensitive ELISA, Alpco Diagnostics, Salem, N.H.) measurements were performed according to the manufacturer's instructions.

### Quantitative Real Time PCR Analysis

Tissues were collected after 6 hours daytime food withdrawal, immediately frozen and stored at −80° C. RNA isolation was performed using Trizol (Invitrogen, Carlsbad, Calif.) according to the manufacturer's protocol. For first strand cDNA synthesis 0.5-1 ng RNA and 5× Script RT Supermix were used (BioRad Laboratories, Herculus, Calif.). Quantitative real time PCR (Q-PCR) was performed using Power SYBR Green PCR master mix (Applied Biosystems, Life Technologies, Grand Island, N.Y.) and samples were analyzed using a ViiA7 PCR machine (Applied Biosystems, Life Technologies, Grand Island, N.Y.). Primers used for Q-PCR were as follows:

| | |
|---|---|
| 36B4 | 5′-cactggtctaggacccgagaa-3′ Seq. ID No. 52; |
| | 5′-agggggagatgttcagcatgt-3′ Seq. ID No. 53 |
| FAS | 5′-ggaggtggtgatag ccggtat-3′ Seq. ID No. 54; |
| | 5′-tgggtaatccatagagcccag-3′ Seq. ID No. 55 |
| SCD1 | 5′-ttcttgcgatacactctggtgc-3′ Seq. ID No. 56; |
| | 5′-cgggattgaatgttcttgtcgt-3′ Seq. ID No. 57 |
| Pck1 | 5′-ctgcataacggtctggacttc-3′ Seq. ID No. 58; |
| | 5′-cagcaactgcccgtactcc-3′ Seq. ID No. 59 |
| G6pc | 5′-cgactcgctatctccaagtga-3′ Seq. ID No. 60; |
| | 5′-gttgaaccagtctccgacca-3′ Seq. ID No. 61 |
| ACC1 | 5′-atgtctggcttgcacctagta-3′ Seq. ID No. 62; |
| | 5′-ccccaaagcgagtaacaaattct-3′ Seq. ID No. 63 |
| TNF | 5′-ccctcacactcagatcatcttct-3′ Seq. ID No. 64; |
| | 5′-gctacgacgtgggcta cag-3′ Seq. ID No. 65 |
| IL- | 5′-gcaactgttcctgaactcaact-3′Seq. ID No. 66; |
| 1β | 5′-atcttttggggtccgtcaact-3′ Seq. ID No. 67 |
| IL-6 | 5′-acaacc acggccttccctactt-3′ Seq. ID No. 68; |
| | 5′-cacgatttcccagagaacatgtg-3′ Seq. ID No. 69 |
| CCL2 | 5′-catccacgtgttggctca-3′ Seq. ID No. 70; |
| | 5′-gatcatcttgctggtgaatgagt-3′ Seq. ID No. 71 |
| CXCL1 | 5′-gactccagccacactccaac-3′ Seq. ID No. 72; |
| | 5′-tgacagcgcagctcattg-3′ Seq. ID No. 73 |
| F4/80 | 5′-tgactcaccttgtggtcctaa-3′ Seq. ID No. 74; |
| | 5′-cttcccagaatccagtctttcc-3′ Seq. ID No. 75 |
| CD68 | 5′-tgtctgatcttgctaggaccg-3′ Seq. ID No. 76; |
| | 5′-gagagtaacggcctttttgtga-3′ Seq. ID No. 77 |
| TBP | 5′-agaacaatccagactagcagca-3′ Seq. ID No. 78; |
| | 5′-gggaacttcacatcacagctc-3′ Seq. ID No. 79 |

### Statistical Analysis

Results are presented as the mean±SEM. Statistical significance was determined by repeated measures ANOVA or student's t test. * denotes significance at p<0.05, **denotes significance at p<0.01.

### Anti-aP2/Glucagon/aP2 Protein Complex Monoclonal Antibody Development and Screening

Obesity is associated with increased levels of circulating aP2, which contributes to the elevation of hepatic glucose production and reduced peripheral glucose disposal and insulin resistance, characteristics of type 2 diabetes. Therefore, neutralizing serum aP2 or disrupting the glucagon/aP2 protein complex from activating the glucagon receptor represents an efficient approach to treat diabetes and possibly other metabolic diseases.

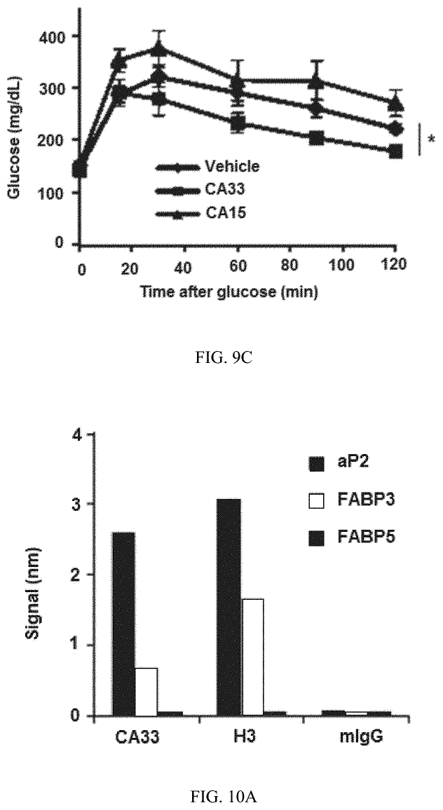

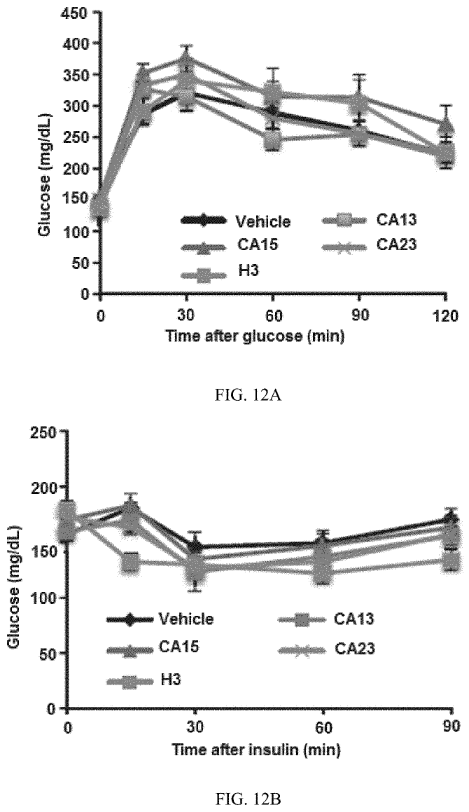

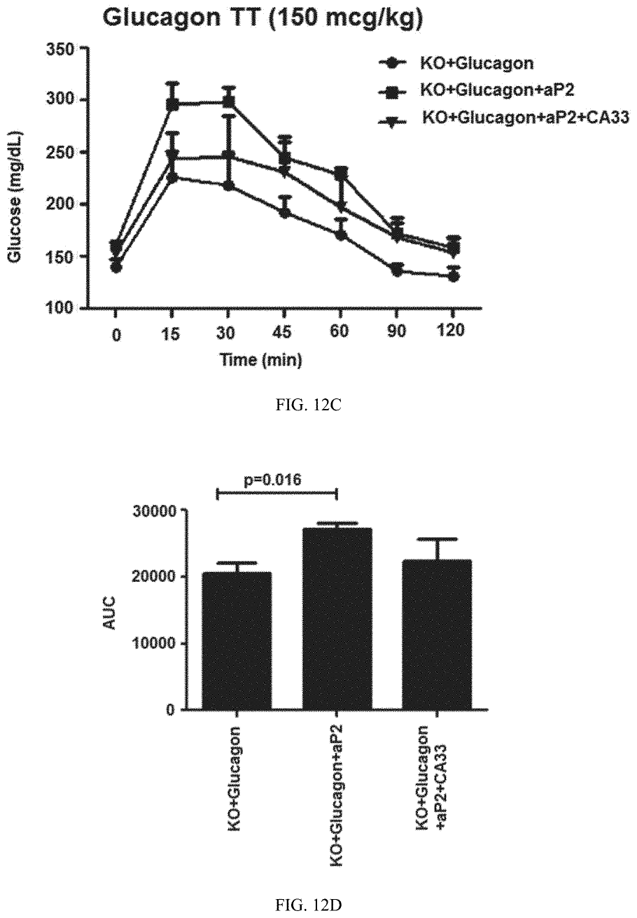

Mouse and rabbit-mouse hybrid monoclonal antibodies raised against the human and mouse aP2 peptides were produced and screened. Assessment of binding affinity by biomolecular interaction analysis using a Biacore system demonstrated a wide range of affinities for these antibodies, from the micromolar to the low nanomolar range (FIG. 9A). Interestingly, CA33 also significantly decreased fasting blood glucose (FIG. 9B), while the other antibodies tested did not improve glycemia. Without wishing to be bound to any one theory, this indicates that CA33 reduced insulin resistance associated with HFD and improved glucose metabolism. The systemic improvement in glucose metabolism was further verified using a glucose tolerance test (GTT). CA33 therapy resulted in significantly improved glucose tolerance (FIG. 9C), while the other antibodies did not improve glucose tolerance and glucose disposal curves were not different compared to vehicle (FIG. 12A). Furthermore, only CA33 treatment markedly improved insulin sensitivity as demonstrated in insulin tolerance tests, while other antibodies tested were similar to vehicle (FIG. 12B). Additionally, aP2 administration to aP2 knockout mice with glucagon rescues glucagon unresponsiveness and preincubation with CA33 with aP2 prevents that (FIGS. 12C and 12D). Taken together and without wishing to be bound to any one theory, these results demonstrated that CA33 uniquely possessed anti-diabetic properties.

CA33 is a Low-Affinity Antibody that Neutralizes aP2/Glucagon/aP2 Protein Complex Activity

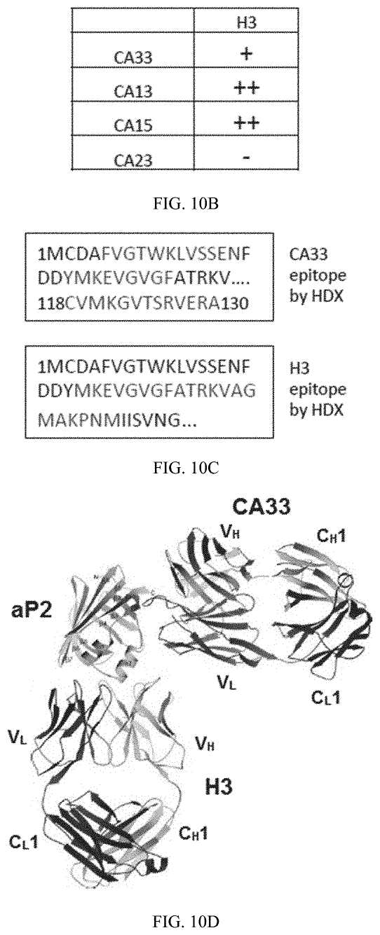

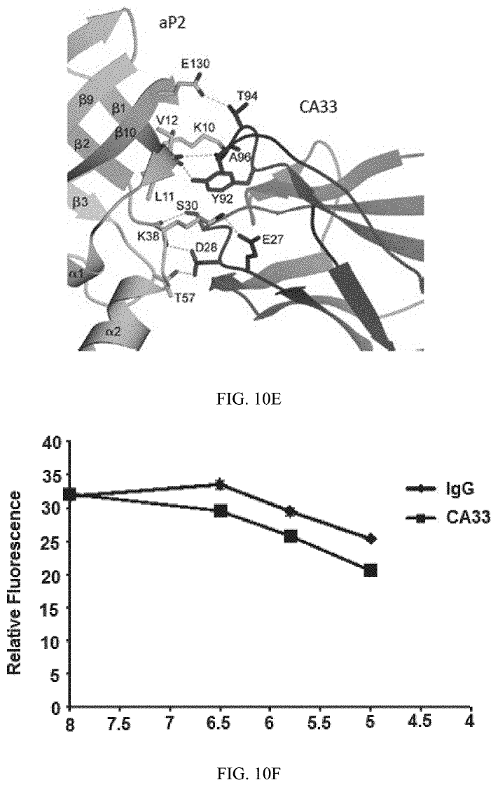

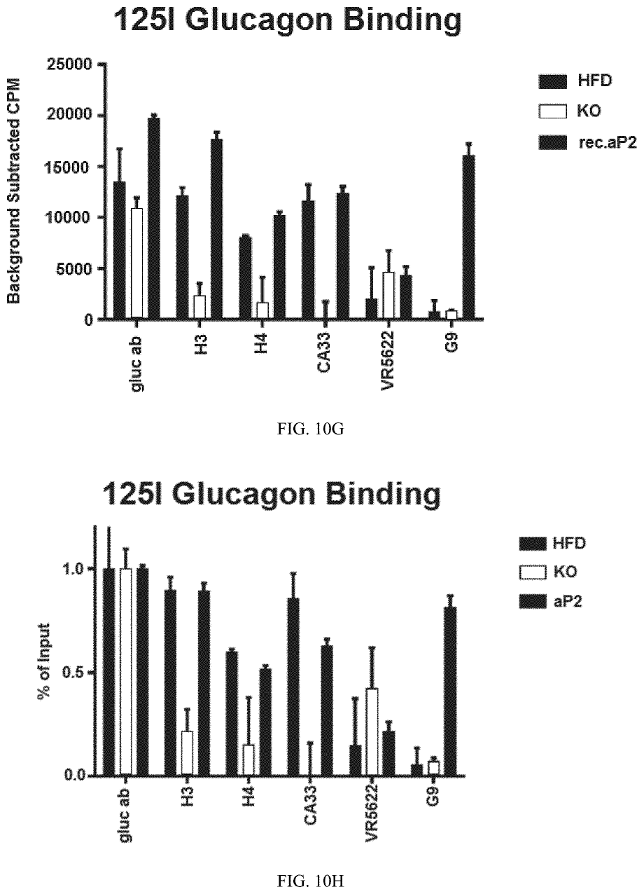

CA33 was further characterized to better understand its unique therapeutic properties. In an octet-binding assay, all of the antibodies tested demonstrated saturable binding to aP2. There was a measurable but low interaction with the related protein FABP3 (˜25% of the aP2/FABP4 interaction) and only minor interaction with Mal1/FABP5 that was similar to control IgG (FIG. 10A).

In cross blocking experiments to begin characterizing the target sites, we found that CA33 partially blocked binding of the ineffective mouse antibody H3 to aP2, while H3 binding was completely blocked by the hybrid antibodies CA13 and CA15 (FIG. 10B). In further analysis, epitope identification based on hydrogen-deuterium exchange mass spectrometry experiments, for example, as described by Pandit et al. (2012) J. Mol. Recognit. March; 25(3):114-24 (incorporated herein by reference), indicated interaction of CA33 with first alpha helix and the first beta sheet of aP2 on residues 9-17, 20-28 and 118-132, which partially overlapped with the epitope identified for H3 (FIG. 10C). Co-crystallization of the Fab fragments of CA33 and H3 with aP2 (FIG. 10D) was then conducted. Analyses of the crystals showed that CA33 binds an epitope spread out over the secondary structure elements beta1 and beta10 and the random coil regions linking alpha2 to beta2 and beta3 to beta4, and includes the aP2 amino acids 57T, 38K, 11L, 12V, 10K and 130E (FIG. 10E). Despite the partial blocking of H3 by CA33, we observed that there is in fact no direct overlap of their epitopes. Instead, the significant movement of the region around aP2 Phe58 may partially block binding of one antibody by the other in the competition experiments. In addition, the low affinity of the CA33 Fab can be explained by the crystal structure. Unusually, only one amino acid in the heavy chain of CA33 makes a contact with aP2, and the majority of the contacts are through the light chain (FIGS. 10D and 10E). In contrast, H3-aP2 contact is more conventional, with both Fab chains interacting with aP2. The structure also showed that CA33 does not bind to the ‘lid’ of the β-barrel (14S to 37A), which has been postulated to control access of lipids to the binding pocket or the ‘hinge’ which contains E15, N16, and F17. In addition, it was found that lipid binding (paranaric acid) to aP2 was not substantially altered by the presence of CA33 (FIG. 10F). H3 does bind directly to the ‘lid’ but has limited activity. Binding of CA33 to lipid-bound aP2 or lipid-free aP2 was also examined using biochemical analysis (Biacore). CA33 binds to both lipid-bound aP2 and lipid-free aP2 with the following affinities:

| Mouse aP2 | Kd |
|---|---|
| Lipid-loaded | 9.3 μM |
| De-lipidated | 4.7 μM |

In addition, anti-mouse IgG SPA beads were incubated with serum from wild-type or aP2 knockout mice with ¹²⁵I glucagon, which shows that aP2 interacts with ¹²⁵I glucagon in physiologically relevant conditions/animal sera (FIGS. 10G and 10H). Without wishing to be bound to any one theory, these results suggest that CA33 activity may be independent of aP2 lipid binding.

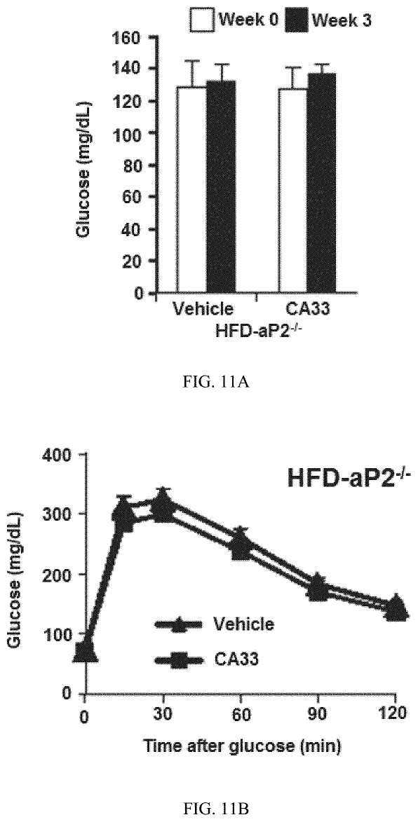

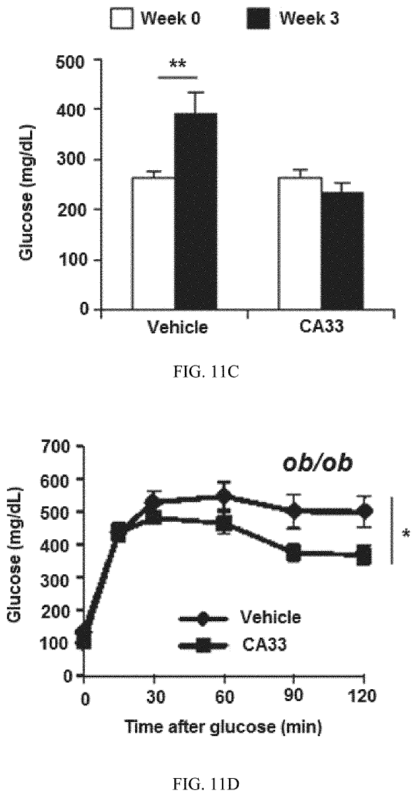

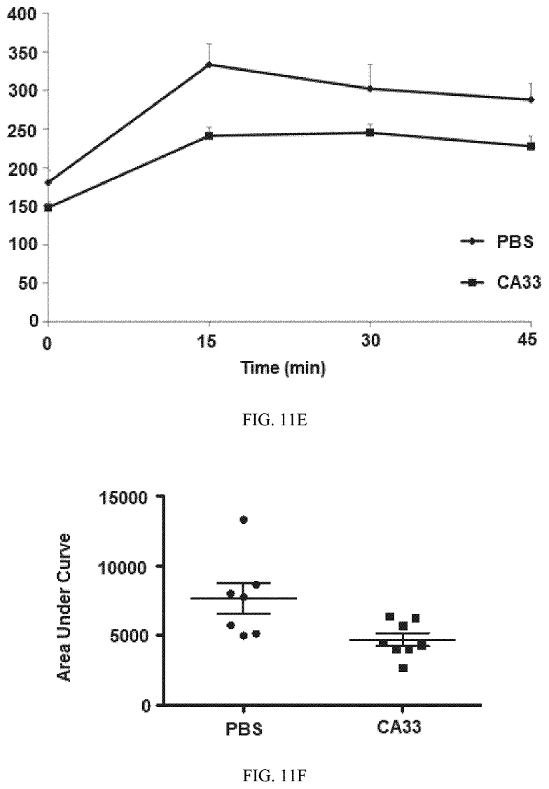

Given the relatively low affinity of CA33 for aP2, off-target effects were examined. The effect of CA33 treatment in aP2−/− mice fed a HFD were tested. In these experiments, antibody therapy failed to induce any change in weight or fasting glucose in this model (FIG. 11A). Furthermore, CA33 did not affect glucose tolerance in obese aP2−/− mice (FIG. 11B), clearly demonstrating that the antibody's effects are due to targeting aP2.

Finally, the effect of CA33 in a second model of severe genetic obesity and insulin resistance using leptin-deficient ob/ob mice was examined. Strikingly, hyperglycemia in the ob/ob mice was normalized in CA33-treated mice compared to controls (FIG. 11C). Normal glucose and lower insulin levels suggest improved glucose metabolism upon neutralization of aP2. Indeed, following administration of exogenous glucose, CA33 treated ob/ob mice also exhibited significantly improved glucose tolerance compared to vehicle treated mice despite the presence of massive obesity (FIG. 11D). It was also shown that CA33 treatment blunts the glucagon response in ob/ob mice after 3 weeks of treatment (FIGS. 11E and 11F) and mimics aP2 deficiency by preventing the actions of glucagon.

### Example 3: Humanization of CA33

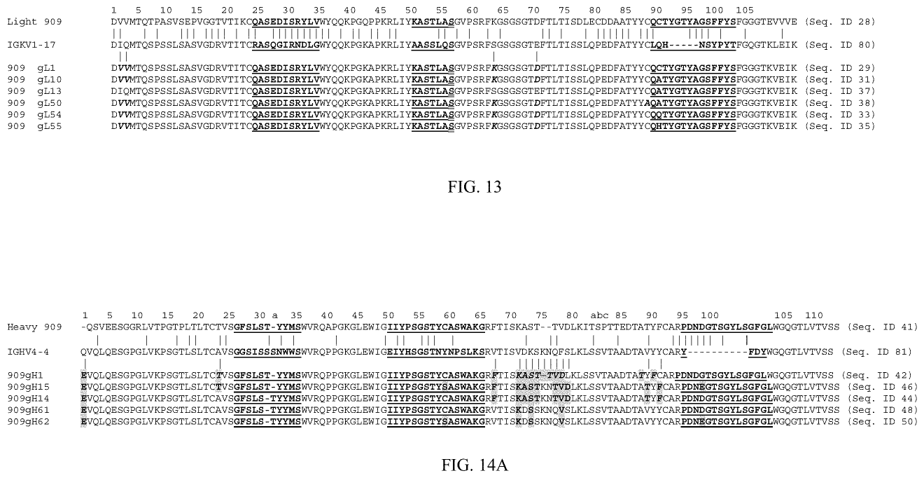

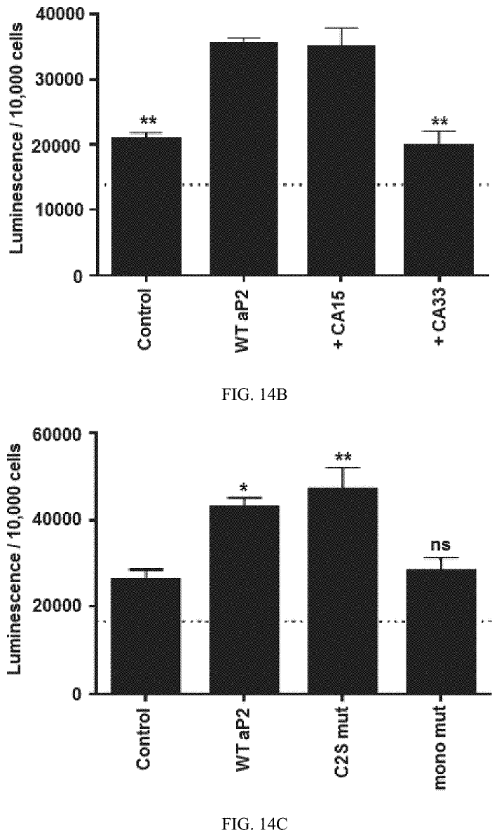

Rabbit Antibody 909 (CA33) was humanized by grafting the CDRs from the rabbit CDR/mouse framework hybrid antibody V-region CDRs onto human germline antibody V-region frameworks. In order to recover the activity of the antibody, a number of framework residues from the rabbit/mouse hybrid V-region were also retained in the humanized sequence. These residues were selected using the protocol outlined by Adair et al. (1991) (Humanized antibodies. WO91/09967). Alignments of the rabbit/mouse hybrid antibody (donor) V-region sequences with the human germline (acceptor) V-region sequences are shown in FIG. 13 (VL) and FIG. 14A (VH), together with the designed humanized sequences. The CDRs grafted from the donor to the acceptor sequence are as defined by Kabat (Kabat et al., 1987), with the exception of CDRH1 where the combined Chothia/Kabat definition is used (see Adair et al., 1991 Humanised antibodies. WO91/09967).

Genes encoding a number of variant heavy and light chain V-region sequences were designed and constructed by an automated synthesis approach by DNA 2.0 Inc. Further variants of both heavy and light chain V-regions were created by modifying the VH and VK genes by oligonucleotide-directed mutagenesis, including, in some cases, mutations within CDRs to modify potential aspartic acid isomerization sites or remove unpaired Cysteine residues. These genes were cloned into vectors to enable expression of humanized 909 IgG4P (human IgG4 containing the hinge stabilizing mutation S241P, Angal et al., Mol Immunol. 1993, 30(1):105-8) antibodies in mammalian cells. The variant humanized antibody chains, and combinations thereof, were expressed and assessed for their potency relative to the parent antibody, their biophysical properties and suitability for downstream processing, leading to the selection of heavy and light chain grafts.

Human V-region IGKV1-17 (A30) plus JK4 J-region was chosen as the acceptor for antibody 909 light chain CDRs. The light chain framework residues in grafts gL1 (Seq. ID No. 29), gL10 (Seq. ID No. 31), gL54 (Seq. ID No. 33) and gL55 (Seq. ID No. 35) are all from the human germline gene, with the exception of residues 2, 3, 63 and 70 (Kabat numbering), where the donor residues Valine (2V), Valine (3V), Lysine (63K) and Aspartic acid (70D) were retained, respectively. Retention of residues 2, 3, 63 and 70 was essential for full potency of the humanized antibody. Residue 90 in CDRL3 of the gL10 graft, gL54 graft, and gL55 graft was mutated from a Cysteine (90C) to a Serine (90S), Glutamine (90Q), and Histidine (H90) residue, respectively, thus removing the unpaired Cysteine residue from the gL10, gL54, and gL55 sequence.

Human V-region IGHV4-4 plus JH4 J-region was chosen as the acceptor for the heavy chain CDRs of antibody 909. In common with many rabbit antibodies, the VH gene of antibody 909 is shorter than the selected human acceptor. When aligned with the human acceptor sequence, framework 1 of the VH region from antibody 909 (Seq. ID No. 41) lacks the N-terminal residue, which is retained in the humanized antibody (FIG. 14A). Framework 3 of the 909 rabbit VH region also lacks two residues (75 and 76) in the loop between beta sheet strands D and E: in graft gH1 (Seq. ID No. 42) the gap in framework 3 is conserved, whilst in graft gH14 (Seq. ID No. 44), gH15 (Seq. ID No. 46), gH61 (Seq. ID No. 48), and gH62 (Seq. ID No. 50) the gap is filled with the corresponding residues (Lysine 75, 75K; Asparagine 76, 76N) from the selected human acceptor sequence (FIG. 14A). The heavy chain framework residues in grafts gH1 and gH15 are all from the human germline gene, with the exception of residues 23, 67, 71, 72, 73, 74, 77, 78, 79, 89 and 91 (Kabat numbering), where the donor residues Threonine (23T), Phenylalanine (67F), Lysine (71K), Alanine (72A), Serine (73S), Threonine (74T), Threonine (77T), Valine (78V), Aspartic acid (79D), Threonine (89T) and Phenylalanine (91F) were retained, respectively. The heavy chain framework residues in graft gH14 are from the human germline gene, with the exception of residues 67, 71, 72, 73 74, 77, 78, 79, 89 and 91 (Kabat numbering), where the donor residues Threonine (23T), Phenylalanine (67F), Lysine (71K), Alanine (72A), Serine (73S), Threonine (74T), Threonine (77T), Valine (78V), Aspartic acid (79D), Threonine (89T) and Phenylalanine (91F) were retained, respectively. The heavy chain framework residues in grafts gH61 and gH62 are from the human germline gene, with the exception of residues 71, 73, and 78 (Kabat numbering), where the donor residues Lysine (71K), Serine (73S), and Valine (78V) were retained, respectively. The Glutamine residue at position 1 of the human framework was replaced with Glutamic acid (1E) to afford the expression and purification of a homogeneous product: the conversion of Glutamine to pyroGlutamate at the N-terminus of antibodies and antibody fragments is widely reported. Residue 59 in CDRH2 (Seq. ID No. 19) of the gH15 graft and gH62 graft was mutated from a Cysteine (59C) to a Serine (59S) residue, thus removing the unpaired Cysteine residue from the gH15 sequence. Residue 98 in CDRH3 (Seq. ID No. 20) of graft gH15 and graft gH62 was mutated from an Aspartic acid (98D) to a Glutamic acid (98E) residue, thus removing a potential Aspartic acid isomerization site from the gH15 sequence.

For expression of humanized Ab 909 in mammalian cells, the humanized light chain V-region gene was joined to a DNA sequence encoding the human C-kappa constant region (K1m3 allotype), to create a contiguous light chain gene. The humanized heavy chain V-region gene was joined to a DNA sequence encoding the human gamma-4 heavy chain constant region with the hinge stabilizing mutation S241P (Angal et al., Mol Immunol. 1993, 30(1):105-8), to create a contiguous heavy chain gene. The heavy and light chain genes were cloned into the mammalian expression vector 1235-pGL3a(1)-SRHa(3)-SRLa(3)-DHFR(3) (Cellca GmbH).

To further examine the downstream signaling events impacted by anti-aP2 antibody inhibition, a human glucagon receptor expressing cAMP reporter system constructed in CHO-K1 cells was utilized. FIG. 14B shows the impact of aP2 inhibition on luciferase activity that was assayed 4 hours post stimulation in CHO-K1 stably transfected with human GCGR-GFP and 4xcAMP-response element and stimulated in the presence of 1 ug/ml aP2, 25 nM glucagon, and 20 ug/ml of CA33 and CA15. FIG. 14C shows that mutating the cysteine residue to serine in aP2 does not impact luciferase activity showing that the effect seen in FIG. 14B is not due to cysteine induced dimerization.

### Example 4: In Vivo Effect of aP2 Neutralization on Glucagon Response

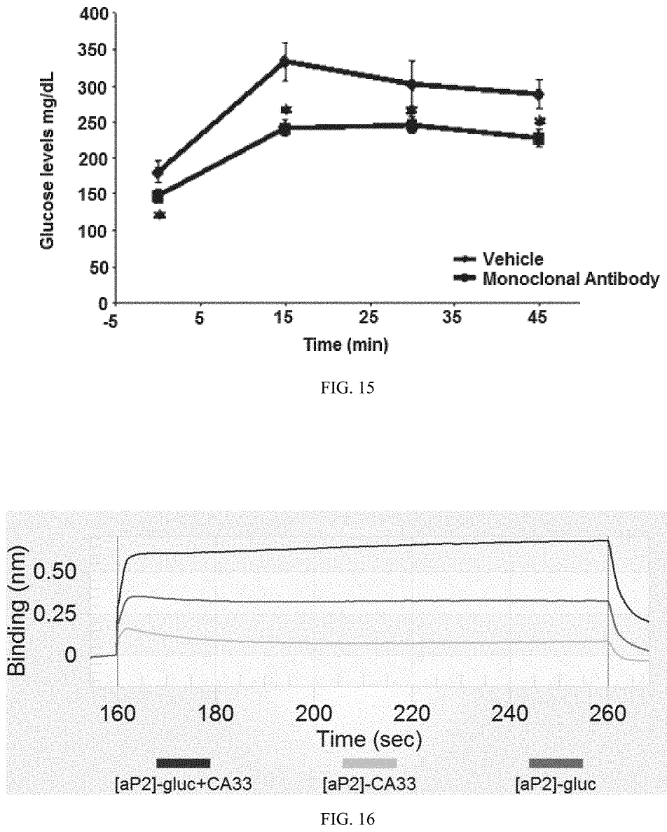

Neutralization of circulating aP2 results in a reduction of glucagon action in mice with diet induced obesity. Mice were fed high fat diet for 20 weeks prior to the experiment. Starting from the 20th week, mice were injected with vehicle or anti-aP2 mAb at a dose of 33 mg/kg injected i.p. twice a week for 3 weeks. At the end of three weeks a glucagon challenge test was performed in which the mice were injected with 150 μg/kg of glucagon after a day time 4-hour fast. The anti-aP2 mAb treated mice showed a significantly lower response to glucagon injection than the vehicle treated mice (FIG. 15). Neutralizing circulating aP2 is thus an effective approach to reducing glucagon activity.

### Example 5: CA33 is Capable of Binding the Glucagon/aP2 Complex

Binding affinity studies were performed using a Blitz instrument (Pall Life Sciences, Menlo Park, Calif.). Biotinylated aP2 was attached to streptavidin probes. Binding affinity of the tethered aP2 was then tested in solutions of glucagon, monoclonal antibody (mAb) (CA33), or glucagon plus mAb (FIG. 16). Glucagon exhibits binding to aP2 on its own and the monoclonal antibody shows a strong binding to the glucagon/aP2 complex. Without wishing to be bound to any one theory, it is possible that this monoclonal antibody's binding to the glucagon/aP2 complex is a key element of the monoclonal antibody's anti-diabetic effect and ability to reduce glucagon action.

### Example 6. Glucagon Treatment Improves aP2 Internalization

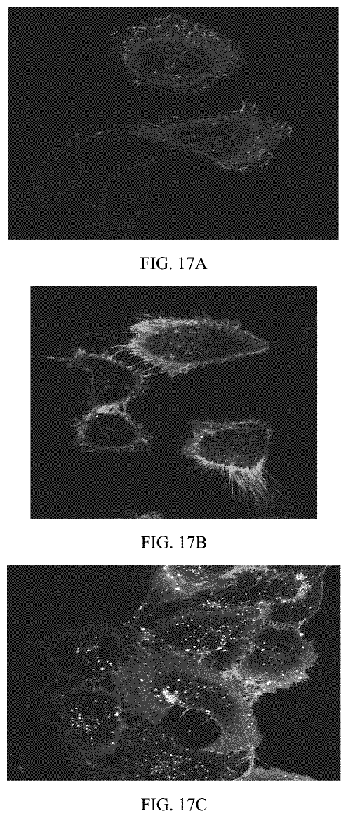

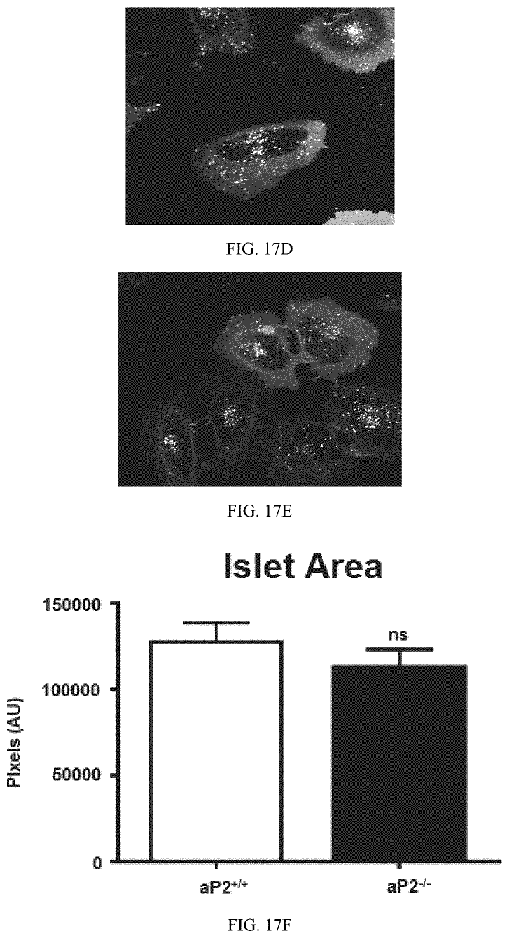

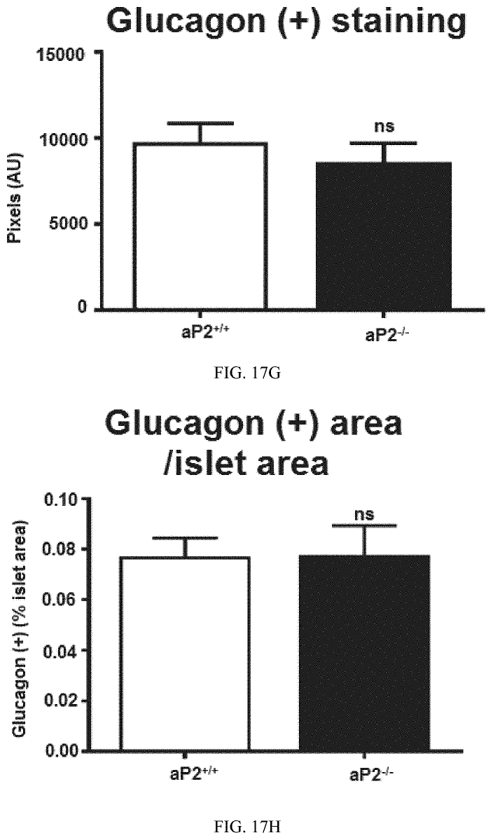

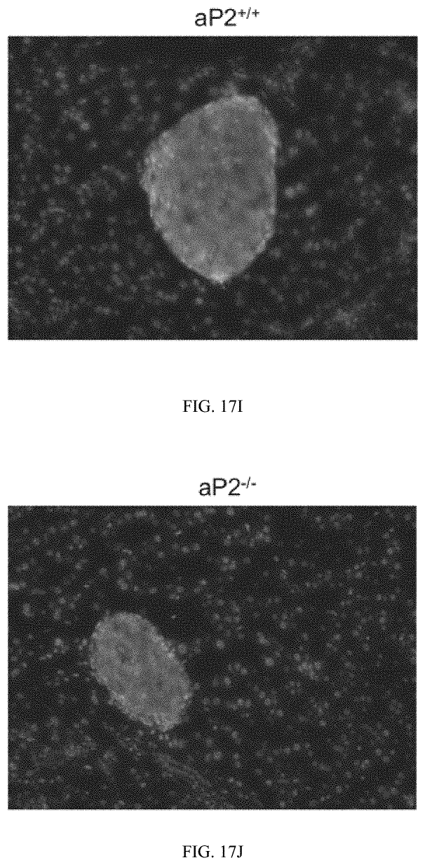

Live-cell imaging was conducted on U2-OS cells transfected with GCGR-GFP following exposure to either aP2 treatment or aP2+ glucagon treatment. As shown in FIG. 17A and FIG. 17B, when cells were not treated with glucagon, minimal internalization of aP2 into cells was observed. When cells were stimulated with glucagon though (FIGS. 17C-17E), internalization of aP2 was greatly increased. Colocalization of the GCGR-GFP signal and the aP2 signal are shown in white in the photos.

### Example 7. aP2 Deficiency is Distinct from Glucagon Receptor Antagonism

A distinguishing feature of glucagon receptor antagonism is alpha cell hyperplasia, but aP2 deficiency does not cause alpha cell hyperplasia as shown in FIGS. 17F-17J. Microscopy images of a cell from the aP2^{+/+} cell line and the aP2^{-/-} cell line, and the islet area of a cell from the aP2^{+/+} cell line and the aP2^{-/-} cell line as measured in pixels was not significantly different. When stained for glucagon, the images of the two cells lines were also not significantly different. Images of the two cells are shown in FIG. 17I (aP2+/+ cell line) and FIG. 17J (aP2^{-/-} cell line).

This specification has been described with reference to embodiments of the invention. However, one of ordinary skill in the art appreciates that various modifications and changes can be made without departing from the scope of the invention as set forth in the claims below. Accordingly, the specification is to be regarded in an illustrative rather than a restrictive sense, and all such modifications are intended to be included within the scope of invention.

What is claimed is:

1.-10. (canceled)

11. A method of identifying a compound capable of neutralizing glucagon/adipocyte binding protein complex (glucagon/aP2) agonism of glucagon receptor (GCGR) comprising:
i. introducing aP2 and glucagon, or glucagon/aP2, into a first cellular assay comprising cells expressing GCGR;
ii. determining a biological activity of GCGR in the cells in the first cellular assay;
iii. introducing aP2 and glucagon, or glucagon/aP2 into a second cellular assay comprising cells expressing GCGR, wherein the aP2 and glucagon, or glucagon/aP2, is introduced in the presence of the compound;
iv. determining a biological activity of GCGR in the cells in the second cellular assay; and,
v. comparing the biological activity of GCGR in the first cellular assay with the biological activity of GCGR in the second cellular assay;
wherein a reduction in GCGR biological activity in the second assay compared to the GCGR biological activity in the first assay is indicative of a compound that neutralizes glucagon/aP2 agonism of GCGR.

12. The method of claim 11, wherein the cell population expressing GCGR is hepatocytes.

13. The method of claim 12, wherein the cell population expressing GCGR is human cells.

14. A method of neutralizing glucagon/aP2 agonism of GCGR in a human comprising administering to the subject a compound that neutralizes the ability of glucagon/aP2 from binding to GCGR.

15. The method of claim 14, wherein the compound is an antibody, antibody fragment, or antigen binding agent.

16.-19. (canceled)

20. A method of treating a human with a disorder mediated by the dysregulation of hepatic glucose production comprising administering to the human a compound that neutralizes the ability of a glucagon/aP2 to agonize GCGR, wherein the compound does not directly bind to GCGR.

21. The method of claim 20, wherein the compound is an antibody, antibody fragment, or antigen binding agent.

22. The method of claim 11, wherein a compound identified as indicative of a compound that neutralizes glucagon/aP2 agonism of GCGR is administered to a human with a disorder mediated by the dysregulation of hepatic glucose production.

23. The method of claim 22, wherein the disorder is nonalcoholic fatty liver disease (NAFLD).

23. The method of claim 22, wherein the disorder is nonalcoholic steatohepatitis (NASH).

24. The method of claim 22, wherein the disorder is selected from the group consisting of diet-induced obesity, type 1 diabetes, type 2 diabetes, hyperglycemia, diabetic ketoacidosis, hyperglycemic hyperosmolar syndrome, cardiovascular disease, diabetic nephropathy, kidney failure, diabetic retinopathy, impaired fasting glucose, impaired glucose tolerance, dyslipidemia, obesity, cataracts, stroke, atherosclerosis, impaired wound healing, hyperglycemia, insulin resistance syndrome, and metabolic syndrome.

25. The method of claim 24, wherein the disorder is type II diabetes.

26. The method of claim 24, wherein the disorder is dyslipidemia.

27. The method of claim 24, wherein the disorder is insulin resistance syndrome.

28. The method of claim 24, wherein the disorder is hyperglycemia.

29. The method of claim 24, wherein the disorder is hyperinsulinemia.


SEQUENCE LISTING (82 sequences)

SEQ ID NO: 1 - protein, 132 aa, Homo sapiens
MCDAFVGTWKLVSSENFDDYMKEVGVGFATRKVAGMAKPNMIISVNGDVITIKSESTFKNTEISFILGQEFDEVTADDRKVKSTITLDGGVLVHVQKWDGKSTTIKRKREDDKLVVECVMKGVTSTRVYERA

SEQ ID NO: 2 - protein, 132 aa, Mus musculus
MCDAFVGTWKLVSSENFDDYMKEVGVGFATRKVAGMAKPNMIISVNGDLVTIRSESTFKNTEISFKLGVEFDEITADDRKVKSIITLDGGALVQVQKWDGKSTTIKRKRDGDKLVVECVMKGVTSTRVYERA

SEQ ID NO: 3 - protein, 11 aa, Homo sapiens
KEVGVGFATRK

SEQ ID NO: 4 - protein, 3 aa, Artificial Sequence
  aP2 fatty acid binding domain amino acid sequence
RVY

SEQ ID NO: 5 - DNA, 399 bp, Homo sapiens
atgtgtgatgcttttgtaggtacctggaaacttgtctccagtgaaaactttgatgattatatgaaagaagtaggagtgggctttgccaccaggaaagtggctggcatggccaaacctaacatgatcatcagtgtgaatggggatgtgatcaccattaaatctgaaagtacctttaaaaatactgagatttccttcatactgggccaggaatttgacgaagtcactgcagatgacaggaaagtcaagagcaccataaccttagatgggggtgtcctggtacatgtgcagaaatgggatggaaaatcaaccaccataaagagaaaacgagaggatgataaactggtggtggaatgcgtcatgaaaggcgtcacttccacgagagtttatgagagagcataa

SEQ ID NO: 6 - DNA, 399 bp, Mus musculus
atgtgtgatgcctttgtgggaacctggaagcttgtctccagtgaaaacttcgatgattacatgaaagaagtgggagtgggctttgccacaaggaaagtggcaggcatggccaagcccaacatgatcatcagcgtaaatggggatttggtcaccatccggtcagagagtacttttaaaaacaccgagatttccttcaaactgggcgtggaattcgatgaaatcaccgcagacgacaggaaggtgaagagcatcataaccctagatggcggggccctggtgcaggtgcagaagtgggatggaaagtcgaccacaataaagagaaaacgagatggtgacaagctggtggtggaatgtgttatgaaaggcgtgacttccacaagagtttatgaaagggcatga

SEQ ID NO: 7 - protein, 11 aa, Artificial Sequence
  CDRL1
QASEDISRYLV

SEQ ID NO: 8 - protein, 7 aa, Artificial Sequence
  CDRL2
KASTLAS

SEQ ID NO: 9 - protein, 14 aa, Artificial Sequence
  CDRL3
QCTYGTYAGSFFYS

SEQ ID NO: 10 - protein, 14 aa, Artificial Sequence
  CDRL3 variant 1
QATYGTYAGSFFYS

SEQ ID NO: 11 - protein, 14 aa, Artificial Sequence
  CDRL3 variant 2
QQTYGTYAGSFFYS

SEQ ID NO: 12 - protein, 14 aa, Artificial Sequence
  CDRL3 variant 3
QHTYGTYAGSFFYS

SEQ ID NO: 13 - protein, 9 aa, Artificial Sequence
  CDRL3 variant 4
QQASHYPLT

SEQ ID NO: 14 - protein, 10 aa, Artificial Sequence
  CDRH1
GFSLSTYYMS

SEQ ID NO: 15 - protein, 10 aa, Artificial Sequence
  CDRH1 variant 1
GYTFTSNAIT

SEQ ID NO: 16 - protein, 16 aa, Artificial Sequence
  CDRH2
IIYPSGSTYCASWAKG

SEQ ID NO: 17 - protein, 16 aa, Artificial Sequence
  CDRH2 variant 1
IIYPSGSTYSASWAKG

SEQ ID NO: 18 - protein, 17 aa, Artificial Sequence
  CDRH2 variant 2
DISPGSGSTTNNEKFKS

SEQ ID NO: 19 - protein, 15 aa, Artificial Sequence
  CDRH3
PDNDGTSGYLSGFGL

SEQ ID NO: 20 - protein, 15 aa, Artificial Sequence
  CDRH3 variant 1
PDNEGTSGYLSGFGL

SEQ ID NO: 21 - protein, 10 aa, Artificial Sequence
  CDRH3 variant 2
LRGFYDYFDF

SEQ ID NO: 22 - protein, 12 aa, Artificial Sequence
  CDRL1 variant 1
SVSSSISSSNLH

SEQ ID NO: 23 - protein, 7 aa, Artificial Sequence
  CDRL2 variant 1
GTSNLAS

SEQ ID NO: 24 - protein, 9 aa, Artificial Sequence
  CDRL3 variant 5
QQWSHYPLT

SEQ ID NO: 25 - protein, 10 aa, Artificial Sequence
  CDRH1 variant 2
GYTFTSNWIT

SEQ ID NO: 26 - protein, 17 aa, Artificial Sequence
  CDRH2 variant 3
DIYPGSGSTTNNEKFKS

SEQ ID NO: 27 - protein, 10 aa, Artificial Sequence
  CDRH3 variant 3
LRGYYDYFDF

SEQ ID NO: 28 - protein, 112 aa, Artificial Sequence
  Rabbit Ab 909 VL-region
DVVMTQTPASVSEPVGGTVTIKCQASEDISRYLVWYQQKPGQPPKRLIYKASTLASGVPSRFKGSGSGTDFTLTISDLECDDAATYYCQCTYGTYAGSFFYSFGGGTEVVVE

SEQ ID NO: 29 - protein, 112 aa, Artificial Sequence
  909 gL1 VL-region
DVVMTQSPSSLSASVGDRVTITCQASEDISRYLVWYQQKPGKAPKRLIYKASTLASGVPSRFKGSGSGTDFTLTISSLQPEDFATYYCQCTYGTYAGSFFYSFGGGTKVEIK

SEQ ID NO: 30 - protein, 219 aa, Artificial Sequence
  909 gL1 VL + CL-region
DVVMTQSPSSLSASVGDRVTITCQASEDISRYLVWYQQKPGKAPKRLIYKASTLASGVPSRFKGSGSGTDFTLTISSLQPEDFATYYCQCTYGTYAGSFFYSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 112 aa, Artificial Sequence
  909 gL10 VL-region
DVVMTQSPSSLSASVGDRVTITCQASEDISRYLVWYQQKPGKAPKRLIYKASTLASGVPSRFKGSGSGTDFTLTISSLQPEDFATYYCQATYGTYAGSFFYSFGGGTKVEIK

SEQ ID NO: 32 - protein, 219 aa, Artificial Sequence
  909 gL10 VL + CL-region
DVVMTQSPSSLSASVGDRVTITCQASEDISRYLVWYQQKPGKAPKRLIYKASTLASGVPSRFKGSGSGTDFTLTISSLQPEDFATYYCQATYGTYAGSFFYSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 112 aa, Artificial Sequence
  909 gL54 VL-region
DVVMTQSPSSLSASVGDRVTITCQASEDISRYLVWYQQKPGKAPKRLIYKASTLASGVPSRFKGSGSGTDFTLTISSLQPEDFATYYCQQTYGTYAGSFFYSFGGGTKVEIK

SEQ ID NO: 34 - protein, 219 aa, Artificial Sequence
  909 gL54 VL + CL-region
DVVMTQSPSSLSASVGDRVTITCQASEDISRYLVWYQQKPGKAPKRLIYKASTLASGVPSRFKGSGSGTDFTLTISSLQPEDFATYYCQQTYGTYAGSFFYSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 112 aa, Artificial Sequence
  909 gL55 VL-region
DVVMTQSPSSLSASVGDRVTITCQASEDISRYLVWYQQKPGKAPKRLIYKASTLASGVPSRFKGSGSGTDFTLTISSLQPEDFATYYCQHTYGTYAGSFFYSFGGGTKVEIK

SEQ ID NO: 36 - protein, 219 aa, Artificial Sequence
  909 gL55 VL + CL-region
DVVMTQSPSSLSASVGDRVTITCQASEDISRYLVWYQQKPGKAPKRLIYKASTLASGVPSRFKGSGSGTDFTLTISSLQPEDFATYYCQHTYGTYAGSFFYSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 112 aa, Artificial Sequence
  909 gL13 VL-region
DIQMTQSPSSLSASVGDRVTITCQASEDISRYLVWYQQKPGKAPKRLIYKASTLASGVPSRFSGSGSGTEFTLTISSLQPEDFATYYCQATYGTYAGSFFYSFGGGTKVEIK

SEQ ID NO: 38 - protein, 219 aa, Artificial Sequence
  909 gL13 VL + CL-region
DIQMTQSPSSLSASVGDRVTITCQASEDISRYLVWYQQKPGKAPKRLIYKASTLASGVPSRFSGSGSGTEFTLTISSLQPEDFATYYCQATYGTYAGSFFYSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 112 aa, Artificial Sequence
  909 gL50 VL-region
DVVMTQSPSSLSASVGDRVTITCQASEDISRYLVWYQQKPGKAPKRLIYKASTLASGVPSRFKGSGSGTDFTLTISSLQPEDFATYYAQATYGTYAGSFFYSFGGGTKVEIK

SEQ ID NO: 40 - protein, 219 aa, Artificial Sequence
  909 gL50 VL + CL-region
DVVMTQSPSSLSASVGDRVTITCQASEDISRYLVWYQQKPGKAPKRLIYKASTLASGVPSRFKGSGSGTDFTLTISSLQPEDFATYYAQATYGTYAGSFFYSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 120 aa, Artificial Sequence
  Rabbit Ab 909 VH region
QSVEESGGRLVTPGTPLTLTCTVSGFSLSTYYMSWVRQAPGKGLEWIGIIYPSGSTYCASWAKGRFTISKASTTVDLKITSPTTEDTATYFCARPDNDGTSGYLSGFGLWGQGTLVTVSS

SEQ ID NO: 42 - protein, 121 aa, Artificial Sequence
  909gH1 VH region
EVQLQESGPGLVKPSGTLSLTCTVSGFSLSTYYMSWVRQPPGKGLEWIGIIYPSGSTYCASWAKGRFTISKASTTVDLKLSSVTAADTATYFCARPDNDGTSGYLSGFGLWGQGTLVTVSS

SEQ ID NO: 43 - protein, 448 aa, Artificial Sequence
  909gH1 IgG4 VH + human ?-4P constant
EVQLQESGPGLVKPSGTLSLTCTVSGFSLSTYYMSWVRQPPGKGLEWIGIIYPSGSTYCASWAKGRFTISKASTTVDLKLSSVTAADTATYFCARPDNDGTSGYLSGFGLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 123 aa, Artificial Sequence
  909gH14 VH region
EVQLQESGPGLVKPSGTLSLTCAVSGFSLSTYYMSWVRQPPGKGLEWIGIIYPSGSTYCASWAKGRFTISKASTKNTVDLKLSSVTAADTATYFCARPDNDGTSGYLSGFGLWGQGTLVTVSS

SEQ ID NO: 45 - protein, 450 aa, Artificial Sequence
  909gH14 IgG4 VH + human ?-4P constant
EVQLQESGPGLVKPSGTLSLTCAVSGFSLSTYYMSWVRQPPGKGLEWIGIIYPSGSTYCASWAKGRFTISKASTKNTVDLKLSSVTAADTATYFCARPDNDGTSGYLSGFGLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 123 aa, Artificial Sequence
  909 gH15 VH region
EVQLQESGPGLVKPSGTLSLTCTVSGFSLSTYYMSWVRQPPGKGLEWIGIIYPSGSTYSASWAKGRFTISKASTKNTVDLKLSSVTAADTATYFCARPDNEGTSGYLSGFGLWGQGTLVTVSS

SEQ ID NO: 47 - protein, 450 aa, Artificial Sequence
  909gH15 IgG4 VH + human ?-4P constant
EVQLQESGPGLVKPSGTLSLTCTVSGFSLSTYYMSWVRQPPGKGLEWIGIIYPSGSTYSASWAKGRFTISKASTKNTVDLKLSSVTAADTATYFCARPDNEGTSGYLSGFGLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 123 aa, Artificial Sequence
  909 gH61 VH region
EVQLQESGPGLVKPSGTLSLTCAVSGFSLSTYYMSWVRQPPGKGLEWIGIIYPSGSTYCASWAKGRVTISKDSSKNQVSLKLSSVTAADTAVYYCARPDNDGTSGYLSGFGLWGQGTLVTVSS

SEQ ID NO: 49 - protein, 450 aa, Artificial Sequence
  909gH61 IgG4 VH + human ?-4P constant
EVQLQESGPGLVKPSGTLSLTCAVSGFSLSTYYMSWVRQPPGKGLEWIGIIYPSGSTYCASWAKGRVTISKDSSKNQVSLKLSSVTAADTAVYYCARPDNDGTSGYLSGFGLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 123 aa, Artificial Sequence
  909 gH62 VH region
EVQLQESGPGLVKPSGTLSLTCAVSGFSLSTYYMSWVRQPPGKGLEWIGIIYPSGSTYSASWAKGRVTISKDSSKNQVSLKLSSVTAADTAVYYCARPDNEGTSGYLSGFGLWGQGTLVTVSS

SEQ ID NO: 51 - protein, 450 aa, Artificial Sequence
  909gH62 IgG4 VH + human ?-4P constant
EVQLQESGPGLVKPSGTLSLTCAVSGFSLSTYYMSWVRQPPGKGLEWIGIIYPSGSTYSASWAKGRVTISKDSSKNQVSLKLSSVTAADTAVYYCARPDNEGTSGYLSGFGLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - DNA, 21 bp, Artificial Sequence
  36B4 Forward
cactggtctaggacccgagaa

SEQ ID NO: 53 - DNA, 21 bp, Artificial Sequence
  36B4 Reverse
agggggagatgttcagcatgt

SEQ ID NO: 54 - DNA, 21 bp, Artificial Sequence
  FAS Forward
ggaggtggtgatagccggtat

SEQ ID NO: 55 - DNA, 21 bp, Artificial Sequence
  FAS Reverse
tgggtaatccatagagcccag

SEQ ID NO: 56 - DNA, 22 bp, Artificial Sequence
  SCD1 Forward
cgggattgaatgttcttgtcgt

SEQ ID NO: 57 - DNA, 22 bp, Artificial Sequence
  SCD1 Reverse
cgggattgaatgttcttgtcgt

SEQ ID NO: 58 - DNA, 21 bp, Artificial Sequence
  Pck1 Forward
ctgcataacggtctggacttc

SEQ ID NO: 59 - DNA, 19 bp, Artificial Sequence
  Pck1 Reverse
cagcaactgcccgtactcc

SEQ ID NO: 60 - DNA, 21 bp, Artificial Sequence
  G6pc Forward
cgactcgctatctccaagtga

SEQ ID NO: 61 - DNA, 20 bp, Artificial Sequence
  G6pc Reverse
gttgaaccagtctccgacca

SEQ ID NO: 62 - DNA, 21 bp, Artificial Sequence
  ACC1 Forward
atgtctggcttgcacctagta

SEQ ID NO: 63 - DNA, 23 bp, Artificial Sequence
  ACC1 Reverse
ccccaaagcgagtaacaaattct

SEQ ID NO: 64 - DNA, 23 bp, Artificial Sequence
  TNF Forward
ccctcacactcagatcatcttct

SEQ ID NO: 65 - DNA, 19 bp, Artificial Sequence
  TNF Reverse
gctacgacgtgggctacag

SEQ ID NO: 66 - DNA, 22 bp, Artificial Sequence
  IL-1b Forward
gcaactgttcctgaactcaact

SEQ ID NO: 67 - DNA, 21 bp, Artificial Sequence
  IL-1b Reverse
atcttttggggtccgtcaact

SEQ ID NO: 68 - DNA, 22 bp, Artificial Sequence
  IL-6 Forward
acaaccacggccttccctactt

SEQ ID NO: 69 - DNA, 23 bp, Artificial Sequence
  IL-6 Reverse
cacgatttcccagagaacatgtg

SEQ ID NO: 70 - DNA, 18 bp, Artificial Sequence
  CCL2 Forward
catccacgtgttggctca

SEQ ID NO: 71 - DNA, 23 bp, Artificial Sequence
  CCL2 Reverse
gatcatcttgctggtgaatgagt

SEQ ID NO: 72 - DNA, 20 bp, Artificial Sequence
  CXCL1 Forward
gactccagccacactccaac

SEQ ID NO: 73 - DNA, 18 bp, Artificial Sequence
  CXCL1 Reverse
tgacagcgcagctcattg

SEQ ID NO: 74 - DNA, 21 bp, Artificial Sequence
  F4/80 Forward
tgactcaccttgtggtcctaa

SEQ ID NO: 75 - DNA, 22 bp, Artificial Sequence
  F4/80 Reverse
cttcccagaatccagtctttcc

SEQ ID NO: 76 - DNA, 21 bp, Artificial Sequence
  CD68 Forward
tgtctgatcttgctaggaccg

SEQ ID NO: 77 - DNA, 22 bp, Artificial Sequence
  CD68 Reverse
gagagtaacggcctttttgtga

SEQ ID NO: 78 - DNA, 22 bp, Artificial Sequence
  TBP Forward
agaacaatccagactagcagca

SEQ ID NO: 79 - DNA, 21 bp, Artificial Sequence
  TBP Reverse
gggaacttcacatcacagctc

SEQ ID NO: 80 - protein, 107 aa, Artificial Sequence
  IGKV1-17
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 81 - protein, 113 aa, Artificial Sequence
  IGHV4-4
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARYFDYWGQGTLVTVSS

SEQ ID NO: 82 - protein, 29 aa, Homo sapiens
HSQGTFTSDYSKYLDSRRAQDFVQWLMNT